(19) United States
(12) Patent Application Publication
(10) Pub. No.: US 2011/0160228 A1
(43) Pub. Date: Jun. 30, 2011

(54) DIHYDRO-ISO-CA-4 AND ANALOGUES: POTENT CYTOTOXICS, INHIBITORS OF TUBULIN POLYMERIZATION

(75) Inventors: Mouâd Alami, Bussy Saint Georges (FR); Samir Messaoudi, Chilly Mazarin (FR); Abdallah Hamze, Massy (FR); Olivier Provot, Sartrouville (FR); Jean-Daniel Brion, Saint Leu La Foret (FR); Jian-Miao Liu, Paris (FR); Jérôme Bignon, Le Val Saint Germain (FR); Joanna Bakala, Paris (FR)

(73) Assignee: CENTRE NATIONAL DE LA RECHERCHE SCIENRIFIQUE (CNRS)

(21) Appl. No.: 12/996,488

(22) PCT Filed: Jun. 4, 2009
(86) PCT No.: PCT/EP09/56885

§ 371 Date: Feb. 23, 2011

(30) Foreign Application Priority Data
Jun. 4, 2008 (FR) 0853694

(51) Int. Cl. A61K 31/52; C07C 43/23; A61K 31/09; A61P 35/00; A61P 17/06; A61K 31/36; C07D 317/54; C07C 69/035; A61K 31/222; A61K 31/136; C07C 217/84; C07C 271/22; A61K 31/27; C07D 209/12; A61K 31/404; A61K 31/26; C07C 255/37; C07D 473/34

(52) U.S. Cl. 514/263.4; 568/660; 514/721; 514/464; 549/445; 560/140; 514/546; 514/646; 564/443; 560/160; 514/476; 548/509; 514/415; 514/514; 558/410; 544/277

(57) ABSTRACT

The present invention relates to compounds of formula (I) below in which: —R₁ and R₃ represent, independently of one another, a methoxy group optionally substituted by one or more fluorine atoms, —R₂ and R₄ represent, independently of one another, a hydrogen atom or a methoxy group optionally substituted by one or more fluorine atoms, —A represents a ring chosen from the group comprising aryl and heteroaryl groups, said ring possibly being substituted by or fused to a heterocycle, —X represents a nitrogen atom or a CH group, and —Z₁ represents a hydrogen atom or a halogen atom, preferably fluorine, and —Z₂ represents a hydrogen atom, a halogen atom, preferably fluorine, a C₁ to C₄ alkyl group, an aryl group or a —CN, —SO₂NR₁₂R₁₃, —SO₂R₉, —COOR₁₅ or —COR₁₅ group, and also to the pharmaceutically acceptable salts thereof, the isomers thereof and the prodrugs thereof.

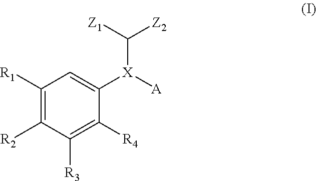

The invention concerns novel compounds inhibiting tubulin polymerization, usable for treating cancer, preparation processes thereof and uses thereof.

Cancer is the major cause of death in the world after cardiovascular diseases. Out of a global total of 58 million deaths recorded in 2005, 7.6 million (13%) were due to cancer. Numerous efforts have been made in the past few years regarding prevention, patient comfort and targeted treatments. The progress in medical oncology is due, in a large part, to understanding the various mechanisms of action in play during cancer, as well as to the development of many cytotoxic medications, possibly combined in polytherapy. For example, we can name cisplatin, anthracyclines, methotrexate, 5 FU, taxoids, irinotecan, etc.

Although surgery and radiation therapy are particularly effective treatments when a cancer is limited to a single region of the body, chemotherapy becomes indispensible when the cancer cells are dispersed. Cytotoxic drugs can be administered before a surgical procedure or radiation treatment to reduce the size of the tumor. They are very often used after these procedures so as to limit metastases and any cancer cells that were resistant to these treatments.

Although many treatments based on cytotoxics have advanced medical research (combination of cytotoxics to prevent resistance phenomena, reduction of adverse effects, improving patient comfort, etc.), anti-tumor chemotherapies need new effective molecules to alleviate the phenomena of resistance to the usual treatments that are increasingly encountered. Furthermore, current drugs used in breast (27.4% of cancer cases in women), lung (13% of cases and soaring in women), prostate (15.5%), colon and rectum (13%) cancers allow reducing the degree of seriousness of tumors without being able to conduct complete cures.

Among the principle cancer drugs used in human therapy, agents interacting with tubulin occupy an important position. Two families of agents can be distinguished:

(a) taxanes, which act by inhibiting cancer cell division, thus inducing their death. They promote tubulin polymerization and stabilization of nonfunctional microtubules and inhibit depolymerization. These include paclitaxel (Taxol®) and docetaxel (Taxotere®). This last is one of the most used chemotherapy agents in the world for treating breast cancer, non-small cell lung cancer and hormone-resistant metastatic prostate cancer.

(b) vinca alkaloids, whose binding with tubulin leads to inhibition of polymerization into microtubules, thus preventing a mitotic spindle from being made. These include vincristine, vindesine, vinblastine and vinorelbine, which make up 10% of the global market in cytotoxic antitumor drugs.

Although they are effective, the use of taxanes and vinca alkaloids is limited by the development of resistance phenomena and the induction of adverse effects, which require routine surveillance. For example, vincristine has sensory-motor nerve toxicity, while hematological toxicity is often the limiting factor in the case of treatment with vinblastine, vindesine or vinorelbine.

Due to the urgency of this situation, the development of new inhibitors has become a major challenge in the past few years. The criteria sought for novel anti-tumor compounds are:

1. the efficacy of the antitumor activity on various strains in in vitro models as well as in in vivo animal models,
2. the emergence of multidrug resistance,
3. the design of original molecules that are watersoluble and, if possible, have a simple chemical structure,
4. the reduction of systemic toxicity, and
5. the identification of the mechanism of action.

In 1982, Pettit et al. (Can. J. Chem. 1982, 60, 1374-1376) isolated combretastatin A-4 (CA-4) from the bark of Combretum caffrum, a South African willow of the Combretaceae family, shown below.

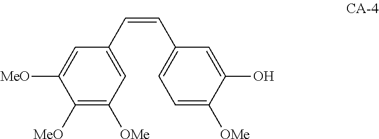

This natural molecule, with an extremely simple structure, is characterized by a Z-configuration stilbene moiety substituted on both aromatic rings by methoxy groups and one hydroxy. The interest of the scientific community in this molecule is particularly linked to antitumor activities thereof (cytotoxic and tubulin polymerization inhibitor).

The first biological evaluations of combretastatin A-4 (CA-4) showed:
a very powerful cytotoxic effect on numerous cell lines, with a nanomolar IC₅₀ (e.g.: IC₅₀=0.9 nM in HCT-15 cells). The cytotoxic activity of CA-4 has also been studied in human umbilical vein endothelial cells (HUVEC) and seems to involve an apoptosis mechanism rather than cellular necrosis;
an antimitotic activity (spindle poison). It binds to tubulin on the colchicine binding site, which inhibits polymerization thereof into microtubules, thus preventing the formation of the mitotic spindle; and
an antiangiogenic activity in vitro by inhibition of endothelial cell proliferation.

However, in vivo, the antitumor activity of CA-4 decreases, or even completely disappears (for example, no antitumor activity is observed in mouse colon adenocarcinoma 26). This reduction in or absence of activity may be partly explained by the low solubility in water due to the lipophilic nature of CA-4, which leads to poor pharmacokinetics in vivo, and, on the other hand, by the ease of isomerization of the double bond of the Z configuration into E. In this regard, it has been demonstrated that the E isomer of CA-4 has a cytotoxic activity on mouse P-388 leukemia cells approximately 60 times lower than the natural Z isomer.

Due to the very simple chemical structure of CA-4 (in comparison to those of vinca alkaloids) and biological activities thereof, numerous studies have been conducted on this compound, currently numbering nearly 500 publications and more than 70 patent applications.

Compounds analogous to CA-4 have been synthesized and evaluated. The molecules CA-4-P, OX14503 and AVE-8062A represented below are currently under development in different laboratories.

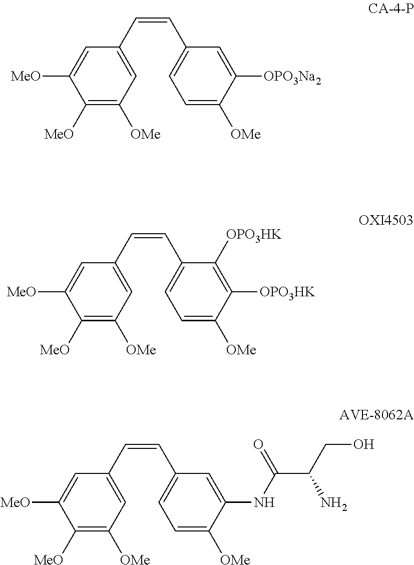

However, they have a double bond with a Z geometry that can lead to the less biologically-active E isomer.

International patent application WO 2006/026 747 and U.S. Pat. No. 5,929,117 describe diphenylethylene derivatives conforming to the following formula:

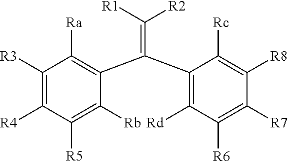

These compounds, which exist in the form of E or Z isomer or a mixture of the two, are described as being tubulin inhibitors. Most of the examples cited are compounds for which the double bond is substituted, in particular, monosubstituted with a CN group (i.e., R1, R2=H, CN). However, no biological test has been done. It is therefore difficult to evaluate the real anticancer potential of these compounds.

The publication by Janendra K. Batra et al. (Molecular Pharmacology 1984, 27, 94-102) is also a study on the tubulin polymerization inhibitor activity of 6-benzyl-1,3-benzodioxoles of the following formula:

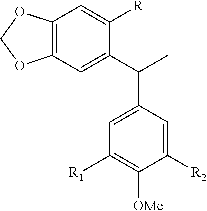

with R₁ and R₂ representing H or OMe. It is apparent that the presence of additional methoxy groups (i.e., when R₁ and/or R₂═OMe) on the phenyl ring would reduce the activity of these benzodioxole derivatives.

Surprisingly, the applicant discovered a new family of compounds derived from CA-4 with strong cytotoxicity (IC₅₀ in the nanomolar range) for a large variety of human cancer cell lines, with inhibition of tubulin polymerization at micromolar concentrations. Moreover, these new compounds have anti-vascular activities.

More precisely, the invention has for subject-matter compounds of the following formula (I):

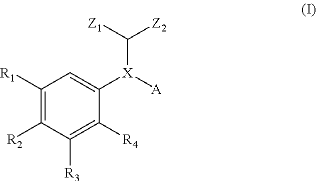

in which:
R₁ and R₃ represent, independently of one another, a methoxy group optionally substituted with one or more fluorine atoms,
R₂ and R₄ represent, independently of one another, a hydrogen atom or a methoxy group optionally substituted with one or more fluorine atoms,
A is a ring chosen from the group including aryls and heteroaryls, said heteroaryls advantageously being chosen from among quinolyl, isoquinolyl, imidazolyl, indolyl, benzothiophenyl, benzofuranyl, benzimidazolyl, purinyl, pyridinyl, pyrrolyl, furanyl and thiophenyl said ring being:
   either fused to a 5- to 7-, and preferably 6-membered heterocycle possibly bearing one or more unsaturations and optionally substituted with one or more C₁ to C₄ alkyl groups and/or with an oxo group (═O)
   or substituted with one or more groups chosen from among halogen atoms, groups —B(OH)₂, C₁ to C₆ alkyls optionally substituted with OH, C₂ to C₄ alkenyls, C₂ to C₄ alkynyls, aryls, heteroaryls, aryloxy, aryl-(C₁ to C₄ alkyl), —COOH, —NO₂, —NR₇R₈, —NHCOR₇, —CONR₇R₈, —NHCOOR₉, —OSi(C₁ to C₄ alkyl)₃, —NHSO₂R₉, C₁ to C₄ alkoxy optionally substituted with one or more fluorine atoms, —OCONR₇R₈, —OSO₂CF₃, —OSO₂R₉, —SO₂R₉, —SO₃R₉, —OSO₃H, —OPO(OR₁₀)₂, —ONR₂R₈, —OR₁₁, —SO₂NR₁₂R₁₃, —SO₂NHR₁₄, —OCOR₁₅, —OCOOR₁₆, —SR₁₇ and a residue of a molecule with antitumor activity bound by means of an ester or amide bond,
      the aryl rings of said groups possibly being substituted with one or more OH, C₁ to C₄ alkoxy, NR₇R₈ groups,
X represents a nitrogen atom or a CH group,
Z₁ represents a hydrogen atom or a halogen atom, preferably fluorine, and
Z₂ represents a hydrogen atom, a halogen atom, preferably fluorine, a C₁ to C₄ alkyl, an aryl or a —CN, —SO₂NR₁₂R₁₃, —SO₃R₉, —COOR₁₅ or —COR₁₅ group, in which:
R₇ and R₈ represent, independently of one another, a hydrogen atom or a C₁ to C₄ alkyl, aryl or heteroaryl group, and advantageously represent a hydrogen atom or a C₁ to C₄ alkyl group,
R₉ represents a C₁ to C₄ alkyl group, aryl or heteroaryl group, and advantageously represents a C₁ to C₄ alkyl group,
R₁₀ represents a hydrogen atom or a C₁ to C₄ alkyl group or a benzyl group,
R₁₁ represents a hydrogen atom, an O-protecting group, a sugar, an amino sugar or an amino acid, the free OH and NH₂ groups of the sugars, amino sugars and amino acids being optionally substituted with an O-protecting and N-protecting group, respectively,
R₁₂ and R₁₃ represent, independently of one another, a hydrogen atom or a C₁ to C₄ alkyl, aryl or heteroaryl group,
R₁₄ represents a —CO—(C₁ to C₄ alkyl) group or the residue of an amino acid molecule bound to the —SO₂NH— group by means of its carboxylic acid function.
R₁₅ represents a hydrogen atom, a C₁ to C₄ alkyl, aryl, or heteroaryl group, or a —(CH₂)ₘCO₂H or —(CH₂)ₘNR₇R₈ group with m representing a whole number comprised between 1 and 3,
R₁₆ represents a C₁ to C₄ alkyl group, aryl, or heteroaryl, or a —(CH₂)ₘCO₂H or —(CH₂)ₘNR₇R₈ group with m representing an integer comprised between 1 and 3, and
R₁₇ represents a hydrogen atom or a C₁ to C₄ alkyl or aryl group,
   as well as pharmaceutically acceptable salts thereof, isomers thereof including enantiomers and isomer mixtures in all proportions, and prodrugs thereof, except for the following compounds:

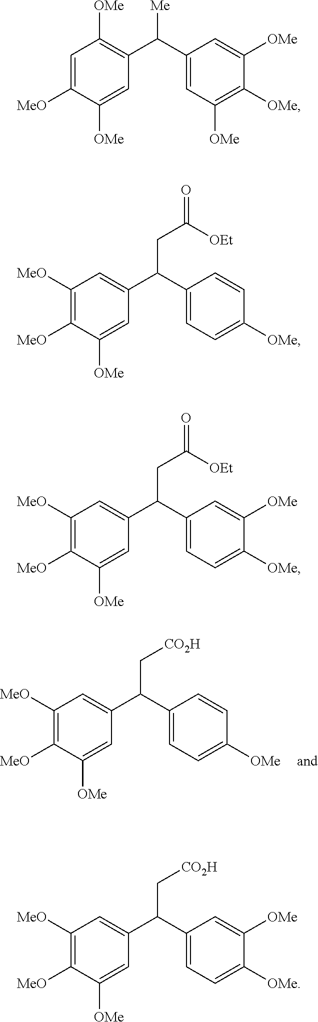

The excluded compounds are described in the following documents: J. K. Batra et al. Molecular Pharmacology 1984, 27, 94-102; Klemm, L. H.; Bower, G. M. J. Org. Chem. 1958, 23, 344-348; Rigby, J. H. et al. J. Org. Chem. 1990, 55, 5078-5088. However, none of these compounds is described as having an anticancer activity.

In the present invention “halogen” means fluorine, chlorine, bromine and iodine atoms. Advantageously, it will be fluorine, bromine and chlorine, and still more advantageously, fluorine.

In the present invention, “C₁ to C₄ alkyl group” means any linear or branched saturated hydrocarbon group with 1 to 4 carbon atoms, in particular methyl, ethyl, n-propyl, isopropyl n-butyl, iso-butyl, sec-butyl, and tert-butyl.

In the present invention, “C₁ to C₆ alkyl group” means any linear or branched saturated hydrocarbon group with 1 to 6 carbon atoms, in particular methyl, ethyl, n-propyl, isopropyl n-butyl, iso-butyl, sec-butyl, tert-butyl, pentyl and hexyl.

In the present invention, “C₂ to C₄ alkenyl group” means any linear or branched hydrocarbon group with 2 to 4 carbon atoms containing at least one double bond, such as a vinyl group (ethenyl).

In the present invention, “C₂ to C₄ alkynyl group” means any linear or branched hydrocarbon group with 2 to 4 carbon atoms containing at least one triple bond, such as an ethynyl or propynyl group.

In the present invention, “C₁ to C₄ alkoxy group” means any linear or branched —O-alkyl group with 1 to 4 carbon atoms, in particular methoxy, ethoxy, propoxy, n-butoxy, iso-butoxy and tert-butoxy groups.

In the present invention, “aryl group” means one or more aromatic rings with 5 to 10 carbon atoms, possibly fused. In particular, the aryl groups can be monocyclic or bicyclic groups, such as, for example, a phenyl or naphthyl group. Advantageously, the aryl group is a phenyl.

In the present invention, “aryloxy group” means any —O-aryl group; aryl group is as defined above. In particular, it may be a phenyloxy group.

In the present invention, “aryl-(C₁ to C₄ alkyl) group” means any aryl group such as defined above bound to the rest of the molecule by means of a C₁ to C₄ alkyl group such as defined above. In particular, it may be a benzyl or phenylethyl group.

In the present invention, “heteroaryl group” means any aromatic group with 5 to 10 cyclic atoms, which are carbon atoms and one or more heteroatoms, such as, for example, sulfur, nitrogen or oxygen atoms. The heteroaryl according to the present invention may consist of one or more fused rings. Preferably, the heteroaryl group will be a quinolyl, isoquinolyl, imidazolyl, indolyl, benzothiophenyl, benzofuranyl, benzimidazolyl, purinyl, pyridinyl, pyrrol or thiophenyl group.

In the present invention, “heterocycle” means any 5- to 7-, and preferably 6-membered saturated or unsaturated nonaromatic hydrocarbon ring, containing one or more heteroatoms, such as, for example, sulfur, nitrogen or oxygen atoms, and preferably containing one heteroatom chosen from among a nitrogen and oxygen atom.

In the scope of the present invention, the group consisting of a fused heterocycle with an aryl group can advantageously be a chromanyl, a chromenyl, a 1,2-dihydroquinolyl or a 1,4-dihydroquinolyl.

In the case where this group is substituted with an oxo group, it is advantageously a group of one of the following formulas:

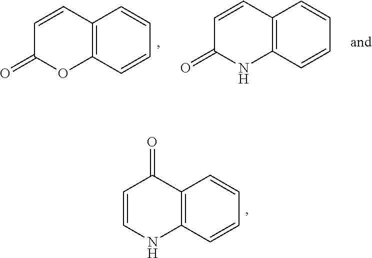

these groups may also be substituted, notably with a C₁ to C₄ alkyl group on the nitrogen atom.

In the present invention, “sugar” means erythrose, threose, ribose, arabinose, xylose, lyxose, allose, altrose, glucose, mannose, gulose, idose, galactose, talose, erythrulose, ribulose, xylulose, psicose, fructose, sorbose or tagatose, in the D or L form. Advantageously, it is glucose, mannose, arabinose or galactose.

In the present invention, “amino sugar” means a sugar in which an amino group replaces a hydroxyl group, such as, for example, glucosamine and galactosamine.

In the present invention, “amino acid” means any natural α-amino acids (for example alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan (Trp), tyrosine (Tyr) and valine (Val) in the D or L form, as well as non-natural amino acids (e.g. (1-naphthyl)alanine (2-naphthyl)alanine, homophenylalanine, (4-chlorophenyl)alanine (4-fluorophenyl)alanine (3-pyridyl)alanine, phenylglycine, diaminopimelic acid, 2,6-diaminoheptane-1,7-dioic acid, 2-aminobutyric acid, 2-aminotetralin-2-carboxylic acid, erythro-β-methylphenylalanine, threo-β-methylphenylalanine, (2-methoxyphenyl)alanine, 1-amino-5-hydroxyindan-2-carboxylic acid, 2-aminoheptane-1,7-dioic acid, (2,6-dimethyl-4-hydroxyphenyl)alanine, erythro-β-methyltyrosine or threo-β-methyltyrosine).

In the present invention, “O-protecting group” means any substituent that protects the hydroxyl or carboxyl group, i.e., a reactive oxygen atom, against undesirable reactions, such as the O-protecting groups described in Greene, “Protective Groups In Organic Synthesis”, (John Wiley & Sons, New York (1981)) and Harrison et al. “Compendium of Synthetic Organic Methods”, Vols. 1 to 8 (J. Wiley & sons, 1971 to 1996). O-protecting groups include methyl or alkyl ethers optionally substituted, for example, methoxymethyl, benzyloxymethyl, 2-methoxyethoxymethyl, 2-(trimethylsilyl)ethoxymethyl, t-butyl, benzyl and triphenylmethyl, benzyl ethers (optionally substituted), tetrahydropyranyl ethers, allyl ethers, substituted ethyl ethers, for example, 2,2,2-trichloroethyl, silyl ethers or alkylsilyl ethers, for example, trimethylsilyl, t-butyldimethylsilyl and t-butyldiphenylsilyl, heterocyclic ethers and esters prepared by reaction of the hydroxyl group with a carboxylic acid, for example tert-butyl, benzyl or methyl esters, carbonates, especially benzyl or haloalkyl carbonate, acetate, propionate, benzoate and the like. Advantageously, it is a tert-butyl, an acetyl or a benzyl.

In the present invention, “N-protecting group” means any substituent that protects the NH₂ group against undesirable reactions, such as the N-protecting groups described in Greene, “Protective Groups In Organic Synthesis”, (John Wiley & Sons, New York (1981)) and Harrison et al., “Compendium of Synthetic Organic Methods”, Vols. 1 to 8 (J. Wiley & sons, 1971 to 1996). N-protecting groups include carbamates, amides, N-alkyl derivatives, amino acetal derivatives, N-benzyl derivatives, imine derivatives, enamine derivatives and N-heteroatom derivatives. In particular, the N-protecting group includes formyl, acetyl, benzoyl, pivaloyl, phenylsulfonyl, benzyl (Bn), t-butyloxycarbonyl (Boc), benzyloxycarbonyl (Cbz), 9-fluorenylmethoxycarbonyl (Fmoc), p-methoxybenzyloxycarbonyl, p-nitrobenzyl-oxycarbonyl, trichloroethoxycarbonyl (TROC), allyloxycarbonyl (Alloc), 9-fluorenylmethoxycarbonyl (Fmoc), trifluoro-acetyl, benzyl carbamates (optionally substituted), and the like. Advantageously, it is an Fmoc group.

“Ester or amide bond” means a —C(O)O— or —C(O)NH— group, respectively. In the particular case of the present invention, the carbonyl of the ester or amide bond will preferentially be bound to the residue of the molecule with the antitumor activity, while the oxygen or NH group of this same bond will be bound to the aryl or heteroaryl group defined under A.

In the present invention, “pharmaceutically acceptable” means what is useful in the preparation of a pharmaceutical composition, what is generally safe, nontoxic and not biologically nor otherwise undesirable and what is acceptable for both veterinary and human pharmaceutical use.

“Pharmaceutically acceptable salts” of a compound means salts that are pharmaceutically acceptable, such as defined here, that have the desired pharmacological activity of the parent compound. Such salts include:

(1) hydrates and solvates,

(2) acid addition salts formed with inorganic acids such as hydrochloric acid, hydrobromic acid, sulfuric acid, nitric acid, phosphoric acid and the like; or formed with organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulfonic acid, trimethylacetic acid, trifluoroacetic acid and the like. Advantageously, it is hydrochloric acid; or

(3) the salts formed when an acidic proton present in the parent compound is replaced with a metal ion, for example an alkaline metal ion, or an alkaline-earth metal ion; or is coordinated with an organic or inorganic base. Acceptable organic bases include diethanolamine, ethanolamine, N-methylglucamine, triethanolamine, tromethamine and the like. Acceptable inorganic bases include aluminum hydroxide, calcium hydroxide, potassium hydroxide, sodium carbonate and sodium hydroxide. Advantageously, the acidic proton is displaced with a Na+ ion, notably by using sodium hydroxide.

Acid addition salts are formed, in particular, with an amine function or with a pyridine. Base addition salts are formed, in particular, with a carboxylic acid (—COOH), phosphate (—OP(O)(OH)₂) or even sulfate (—OSO₃H) function.

In the present invention, “isomers” means diastereoisomers or enantiomers. Therefore, they are isomers of configuration, also called “stereoisomer”. Stereoisomers that are not mirror images of one another are thus designated as “diastereoisomers” and stereoisomers that are mirror images of one another but not superimposable are designated as “enantiomers”, also called “optical isomers”.

A carbon atom bound to four non-identical substituents is called a “chiral centre”. When a molecule has such a chiral centre, it is called chiral and has two enantiomeric forms. When a molecule has several chiral centres, then it has several diastereoisomer and enantiomer forms.

An equimolar mixture of two enantiomers is called a racemic mixture.

In the present invention, “prodrug” means a compound that is administered in an inactive (or less active) form that is metabolized in vivo, particularly by the action of enzymes or gastric acid, into an active (or more active) form. The use of a prodrug particularly improves the physicochemical parameters of a molecule, such as solubility, as well as the pharmacokinetics (vectorization, bioavailability, etc.), in order to improve assimilation thereof by an organism after administration. In particular, a prodrug of a molecule bearing an amino group (NH₂) can result from the acylation or phosphorylation of this amino group. When a molecule bears a hydroxy (OH) group, the prodrug can result in particular from the acylation or phosphorylation of this hydroxy group.

Advantageously, R₄ represents a hydrogen atom.

Advantageously, R₂ represents a methoxy group, optionally substituted with one or more fluorine atoms, and preferably represents a methoxy group.

Advantageously, R₁, R₂ and R₃ represent, independently of one another, a methoxy group, optionally substituted with one or more fluorine atoms, and preferably each represents a methoxy group.

Advantageously, R₄ represents a hydrogen atom and R₁, R₂ and R₃ represent, independently of one another, a methoxy group, optionally substituted with one or more fluorine atoms, and preferably each represents a methoxy group.

Advantageously, Z₂ represents a hydrogen atom, a fluorine atom, a C₁ to C₄ alkyl, —CN, —SO₃R₉, —COOR₁₅ or —COR₁₅ group,

According to one advantageous embodiment, Z₁ represents a hydrogen or halogen atom according to the following conditions:
when Z₁ represents a halogen atom, then Z₂ represents a halogen atom, preferably Z₁ and Z₂ represent the same halogen atom, advantageously fluorine, and
when Z₁ represents a hydrogen atom, then Z₂ represents a hydrogen atom, a C₁ to C₄ alkyl, an aryl or a —CN, —SO₂NR₁₂R₁₃, —SO₃R₉, —COOR₁₅ or —COR₁₅ group, R₉, R₁₂, R₁₃ and R₁₅ being such as defined previously, and Z₂ advantageously represents a hydrogen atom, a fluorine atom, a C₁ to C₄ alkyl, —CN, —SO₃R₉, —COOR₁₅ or —COR₁₅ group, still more advantageously represents a hydrogen atom, an acetyl group or a —CN group, preferably represents a hydrogen atom or a —CN group, and more preferably represents a hydrogen atom.

Advantageously, either Z₁ and Z₂ each represent a fluorine atom, or Z₁ represents a hydrogen atom and Z₂ represents a hydrogen atom or a —CN or —COCH₃ group.

Still more advantageously, Z₁ and Z₂ each represent a hydrogen atom.

Still advantageously, X represents a CH group.

Still advantageously, the molecule with antitumor activity will be a molecule with an antivascular, cytotoxic, antiangiogenic, antiapoptotic or kinase-inhibiting activity. In particular, it be chosen among will from 6-mercaptopurine, fludarabine, cladribine, pentostatin, cytarabine, 5-fluorouracil, gemcitabine, methotrexate, raltitrexed, irinotecan, topotecan, etoposide, daunorubicin, doxorubicin, epirubicin, idarubicin, pirarubicin, mitoxantrone, chlormethine, cyclophosphamide, ifosfamide, melphalan, chlorambucil, busulfan, carmustine, fotemustine, streptozocin, carboplatin, cisplatin, oxaliplatin, procarbazine, dacarbazine, bleomycin, vinblastine, vincristine, vindesine, vinorelbine, paclitaxel, docetaxel, L-asparaginase, flutamide, nilutamide, bicalutamide, cyproterone acetate, triptorelin, leuprorelin, goserelin, buserelin, formestane, aminoglutethimide, anastrazole, letrozole, tamoxifen, octreotide, lanreotide, (Z)-3-[2,4-dimethyl-5-(2-oxo-1,2-dihydro-3-ylidenemethylindol)-1H-pyrrol-3-yl]-propionic acid (SU 6668), 4-((9-chloro-7-(2,6-difluorophenyl)-5H-pyrimidol(5,4-d) (2)benzazepin-2-yl)amino)benzoic acid (MLN-8054), 5,6-dimethylxanthenone-4-acetic acid (DMXAA) or even 3-(4-(1,2-diphenylbut-1-enyl)phenyl)acrylic acid (GW 5638). Advantageously, it is SU 6668, MLN-8054, DMXAA or GW 5638 and even more advantageously DMXAA.

Advantageously, the molecule with antitumor activity will bear a carboxylic acid function COOH, such as SU 6668, MLN-8054, DMXAA or GW 5638, thus allowing coupling to the aryl or heteroaryl group of A, substituted with at least one OH or NH₂ group, by an esterification or amidification reaction. However, a molecule with antitumor activity may be used on which an acid function has been grafted to allow binding with the aryl or heteroaryl group of A.

The amide or ester bond thus formed has the advantage of being able to be easily hydrolyzed in vivo. Thus, after administration of the compound of the invention, the molecule with antitumor activity as well as a novel molecule of the invention can be released, allowing a double therapeutic action.

In one particular embodiment of the invention, A is a ring chosen from the group containing aryl and heteroaryl groups, in particular phenyl, naphthyl and indolyl groups, and preferably phenyl, said ring can be:
either fused to a 5- to 7-, and preferably 6-membered heterocycle possibly bearing one or more unsaturations and optionally substituted with one or more C₁ to C₄ alkyl groups and/or with an oxo group,
or substituted with one or more groups chosen from among halogen atoms, —B(OH)₂, C₁ to C₄ alkyls, C₂ to C₄ alkenyls, C₂ to C₄ alkynyls, aryls, heteroaryls, —COOH, —NO₂, —NR₇R₈, —NHCOR₇, —CONR₇R₈, —NHCOOR₉, —OSi(C₁-C₄ alkyl)₃, —NHSO₂R₉, C₁ to C₄ alkoxy optionally substituted with one or more fluorine atoms, —OCONR₇R₈, —OSO₂CF₃—OSO₂R₉, —SO₂R₉, —SO₃R₉, —OSO₃H, —OPO(OR₁₀)₂, —ONR₇R₈, —OR₁₁, —SO₂NR₁₂R₁₃, —SO₂NHCOR₁₄, —OCOR₁₅, —OCOOR₁₆, —SR₁₇ and a residue of a molecule with antitumor activity bound by means of an ester or amide bond, R₇, R₈, R₉, R₁₀, R₁₁, R₁₂, R₁₃, R₁₄, R₁₅, R₁₅ and R₁₇ being such as defined above.

In another particular embodiment of the invention, A is a ring chosen from the group containing phenyl, naphthyl, purinyl, benzofuranyl, pyridinyl, quinolyl and indolyl groups, said ring can be:
either fused to a 6- to 7-, and preferably 6-membered heterocycle possibly bearing one or more unsaturations and optionally substituted with one or more C₁ to C₄ alkyl groups and/or with an oxo group (═O),
or substituted with one or more groups chosen from among halogen atoms, —B(OH)₂, C₁ to C₆ alkyls optionally substituted with OH, C₂ to C₄ alkenyls, C₂ to C₄ alkynyls, aryls, heteroaryls, aryloxy, aryl-(C₁ to C₄ alkyl), —COOH, —NO₂, —NR₇R₈, —NHCOR₇, —CONR₇R₈, —NHCOOR₉, —OSi(C₁ to C₄ alkyl)₃, —NHSO₂R₉, C₁ to C₄ alkoxy optionally substituted with one or more fluorine atoms, —OCONR₇R₈, —OSO₂CF₃, —OSO₂R₉, —SO₂R₉, —SO₃R₉, —OSO₃H, —OPO(OR₁₀)₂, —ONR₇R₈, —OR₁₁, —SO₂NR₁₂R₁₃, —SO₂NHR₁₄, —OCOR₁₅, —OCOOR₁₆, —SR₁₇ and a residue of a molecule with antitumor activity bound by means of an ester or amide bond,
   the aryl rings of said groups possibly being substituted with one or more OH, C₁ to C₄ alkoxy, NR₇R₈ groups,
   R₇, R₈, R₉, R₁₀, R₁₁, R₁₂, R₁₃, R₁₄, R₁₅, R₁₆ and R₁₇ being such as defined above.

In another particular embodiment of the invention, A is a ring chosen from the group containing aryl, quinolyl, isoquinolyl, imidazolyl, indolyl, benzothiophenyl, benzofuranyl, benzimidazolyl, purinyl, pyridinyl, pyridazinyl, pyrrolyl, furanyl and thiophenyl groups, and in particular containing phenyl, naphthyl, purinyl, benzofuranyl, pyridinyl, quinolyl and indolyl groups,

said ring can be substituted with one or more groups chosen from among halogen atoms, —B(OH)₂, C₁ to C₆ alkyls optionally substituted with OH, C₂ to C₄ alkenyls, C₂ to C₄ alkynyls, aryls, heteroaryls, aryloxy, aryl-(C₁ to C₄ alkyl), —COOH, —NO₂, —NR₇R₈, —NHCOR₇, —CONR₇R₈, —NHCOOR₉, —OSi(C₁ to C₄ alkyl)₃, —NHSO₂R₉, C₁ to C₄ alkoxy optionally substituted with one or more fluorine atoms, —OCONR₇R₈, —OSO₂CF₃, —OSO₂R₉, —SO₂R₉, —SO₃R₉, —OSO₃H, —OPO(OR₁₀)₂, —ONR₇R₈, —OR₁₁, —SO₂NR₁₂R₁₃, —SO₂NHR₁₄, —OCOR₁₅, —OCOOR₁₆, —SR₁₇ and a residue of a molecule with antitumor activity bound by means of an ester or amide bond, and notably chosen from among —SO₂R₉ and OR₁₁,
the aryl rings of said groups possibly being substituted with one or more OH, C₁ to C₄ alkoxy groups, NR₇R₈ groups,
R₇, R₈, R₉, R₁₀, R₁₁, R₁₂, R₁₃, R₁₄, R₁₅, R₁₆ and R₁₇ being such as defined above.

Advantageously, A is a ring chosen from the group containing aryls and heteroaryls and more particularly phenyl, naphthyl, quinolyl, isoquinolyl, imidazolyl, indolyl, benzothiophenyl, benzofuranyl, benzimidazolyl, purinyl, pyridinyl, pyridazinyl, pyrimidyl, pyrazinyl, pyrrolyl, furanyl and thiophenyl groups, in particular phenyl, naphthyl, purinyl, benzofuranyl, pyridinyl, quinolyl and indolyl groups, said ring being able to be substituted with one or more groups chosen from among -Me, -Bn, —C₆H₄—OMe, —CH₂—C₆H₄—OMe, —(CH₂)₂—C₆H₄—OMe, —(CH₂)₂—C₆H₂—(OMe)₃, —OH, —OMe, —OBn —OCOMe, —C₆H₄NH₂, —OC₆H₄NH₂, —NH₂, —OCONEt₂, —(CH₂)ₓ—OH with x=3, 4, 5 or 6, —OCOCH₂NME₂, —OPO₃H₂, —F and

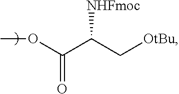

or can be fused to a heterocycle of the formula

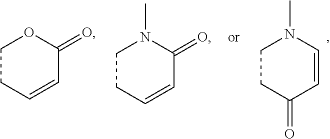

the dashed bond representing the common bond between the heterocycle and said ring.

Advantageously, the compounds of the invention conform to the following formula (Ia):

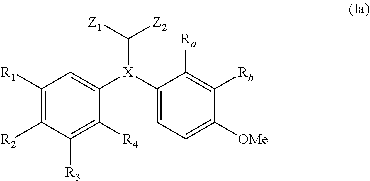

or to a pharmaceutically acceptable salt, an isomer or a prodrug thereof, in which:

R₁, R₂, R₃, R₄, X, Z₁ and Z₂ are such as defined for the compound of formula (I),

Rₐ represents a hydrogen or halogen atom, or a —B(OH)₂, C₁ to C₄ alkyl, C₂ to C₄ alkenyl, C₂ to C₄ alkynyl, aryl, heteroaryl, —COOH, —NO₂, —NR₇R₈, —NHCOR₇, —CONR₇R₈, —NHCOOR₉, —OSi(C₁-C₄ alkyl)₃, —NHSO₂R₉, C₁ to C₄ alkoxy optionally substituted with one or more fluorine atoms, —OCONR₇R₈, —OSO₂CF₃, —OSO₂R₉, —SO₂R₉, —SO₃R₉, —OSO₃H, —OPO(OR₁₀)₂, —ONR₇R₈, —OR₁₁, —SO₂NR₁₂R₁₃, —SO₂NHCOR₁₄, —OCOR₁₅, —OCOOR₁₆ or —SR₁₇ groups,

—R_{b} represents a halogen atom, and preferably a fluorine atom, an aryloxy, —OR₁₁, —OCOR₁₅, —OCOOR₁₅, —OCONR₇R₈, —OSO₂R₉, —OSO₂CF₃, —OSO₃H, —OPO(OR₁₀)₂, —ONR₇R₈, —NR₇R₈, —NHCOR₇, —NHCOOR₉ or —NHSO₂R₉ group or a residue of an antivascular molecular bound by means of an ester or amide bond,

the aryl rings of said groups Rₐ, R_{b} possibly being substituted with one or more OH, C₁ to C₄ alkoxy groups, NR₇R₈ groups,
R₇, R₈, R₉, R₁₀, R₁₁, R₁₂, R₁₃, R₁₄, R₁₅, R₁₆ and R₁₇ being such as defined above.

Advantageously, Rₐ represents a hydrogen atom or a —NR₇R₈, —NHCOR₇, —CONR₇R₈, —NHCOOR₉, —NHSO₂R₉, —OCONR₇R₈, —OSO₂CF₃, —OSO₂R₉, —OSO₃H, —OPO(OR₁₀)₂, —ONR₇R₈, —SO₃R₉, —SO₂NR₁₂R₁₃, —SO₂NHCOR₁₄, —OCOR₁₅ or —OCOOR₁₆ group, with R₇, R₈, R₉, R₁₀, R₁₁, R₁₃, R₁₄, R₁₅, R₁₆ and R₁₇ being such as defined above.

Even more advantageously, Rₐ represents a hydrogen atom or a —NR₇R₈, —NHCOR₇, —CONR₇R₈, —NHCOOR₉, —OCONR₇R₈, —OPO(OR₁₀)₂, —OCOR₁₅ or —OCOOR₁₆ group, with R₇, R₈, R₉, R₁₀, R₁₅, and R₁₆ being such as defined above.

Even more advantageously, Rₐ represents a hydrogen atom.

In particular, the compounds of the invention can be chosen from among:

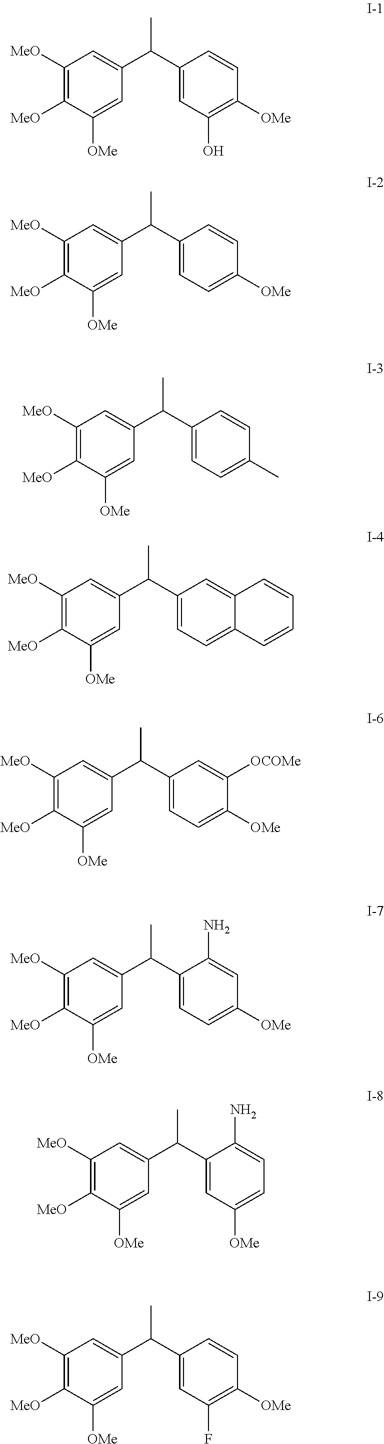

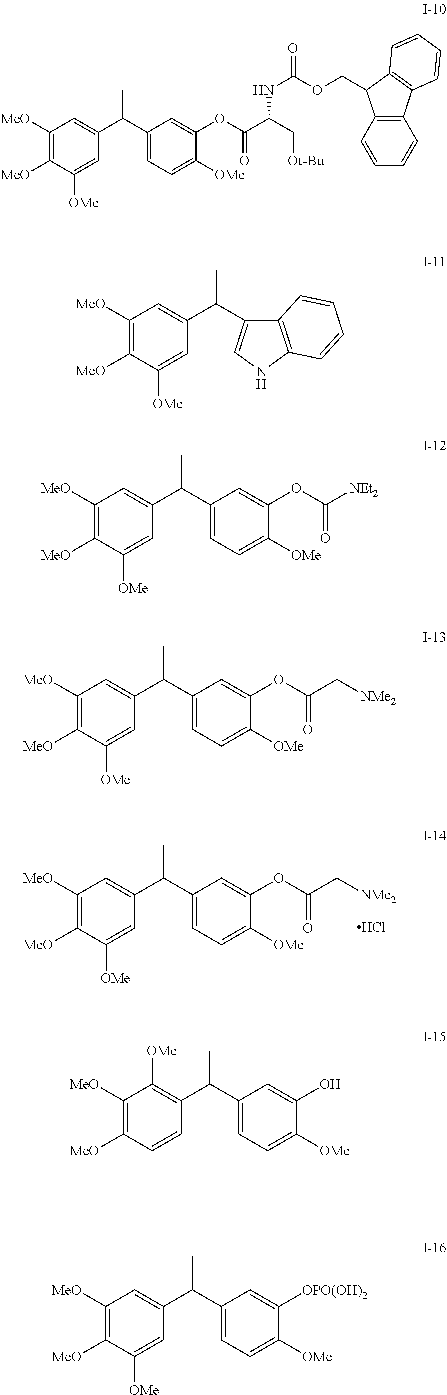

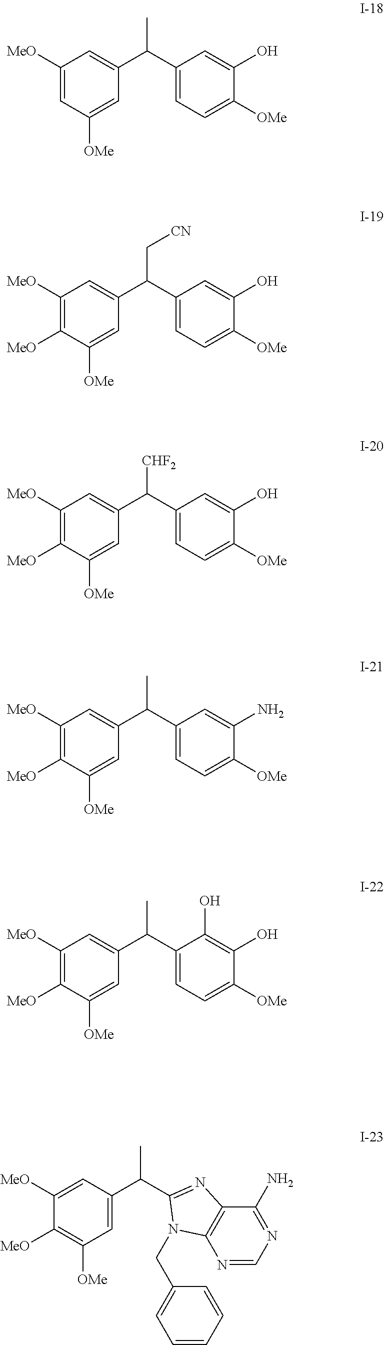

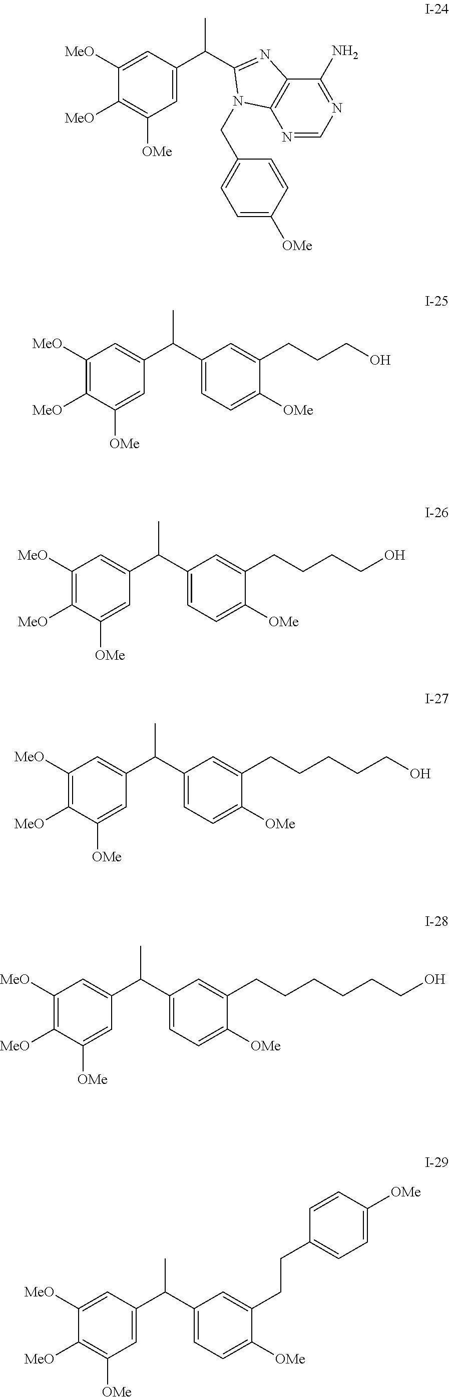

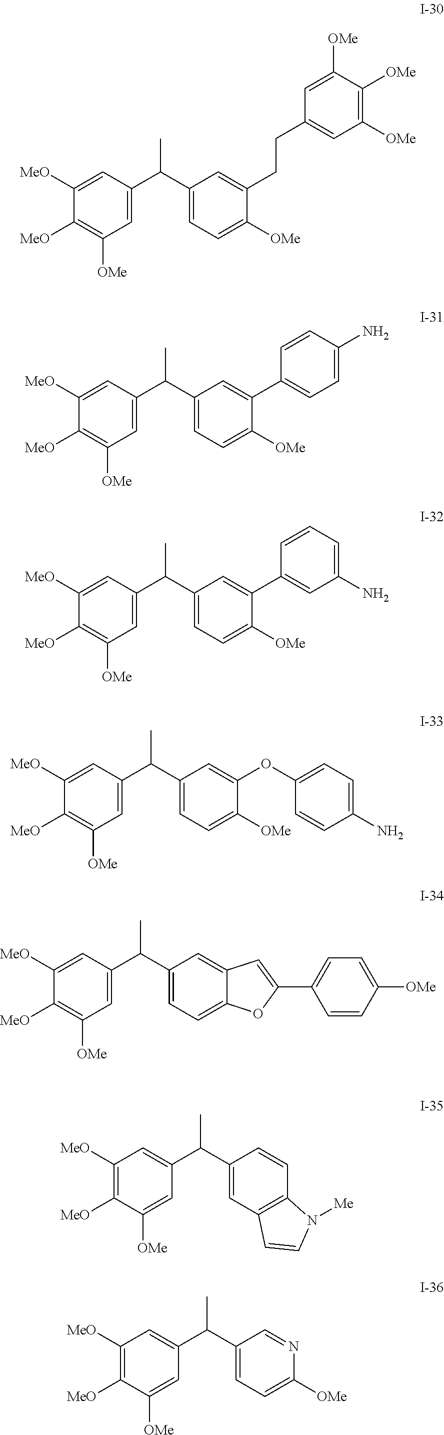

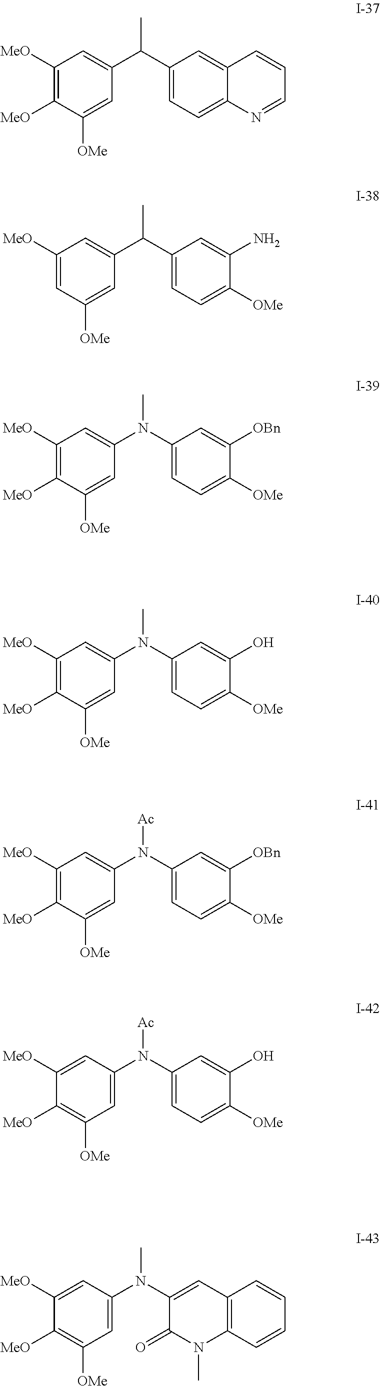

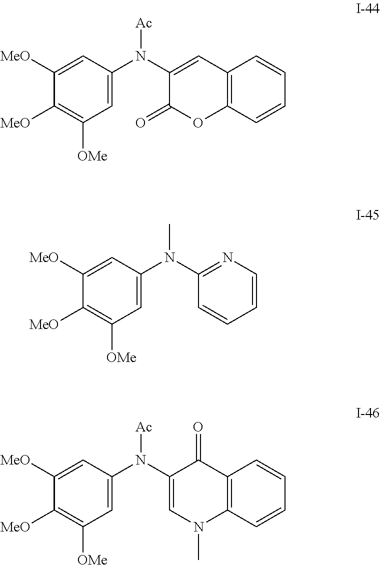

The absence of a ethylene double bound of compounds of formula (I) finally resolves the problem of the isomerization that can occur in vivo, leading to reduction in (or absence of) cytotoxic activity, as is the case for CA-4, for example.

The invention also has for a subject matter, the synthesis processes of compounds of formula (I).

The compounds of formula (I) can be synthesized according to processes known to the person skilled in the art, from products available commercially or prepared according to methods known to the person skilled in the art.

In particular, compounds of formula (I) in which X represents a CH group can be prepared by hydrogenation of the double bond of a compound of formula (II) below:

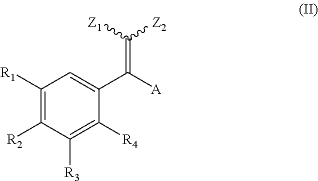

in which R₁, R₂, R₃, R₄, A, Z₁ and Z₂ are such as defined previously for the compound of formula (I), then separation from the reaction medium of compound (I) thus obtained.

This step may be followed by possible additional conventional steps for modification of the A and possibly Z₂ substituents.

The compound thus obtained can be separated from the reaction medium by methods well-known to the person skilled in the art, such as, for example, by extraction, evaporation of the solvent or even by precipitation and filtration.

The compound can also be purified, if necessary, by techniques well known to the person skilled in the art, such as by recrystallization if the compound is crystalline, by distillation, by silica gel column chromatography or even by high performance liquid chromatography (HPLC).

Hydrogenation is conducted under hydrogen atmosphere, particularly in the presence of palladium on carbon (Pd/C) as a catalyst or possibly PtO₂. Advantageously, 5 to 30 mol %, preferably approximately 10 mol % of catalyst are used during this reaction. Furthermore, ethyl acetate will advantageously be used as the solvent during this step.

According to a first variant, the compound of formula (II), for which Z₁ represents a hydrogen atom and Z₂ represents a hydrogen atom, a C₁ to C₄ alkyl or an aryl, can be prepared according to the following successive steps:
reaction of a compound of formula (III) below:

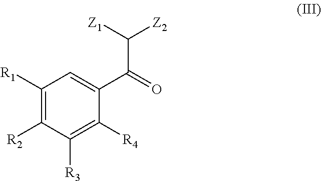

in which R₁, R₂, R₃, and R₄, are such as defined previously, and Z₁ and Z₂ are such as defined above in the context of this first variant, with an organometallic compound of formula A-M in which A is such as defined previously and M represents an alkaline metal or alkaline-earth metal substituted with a halogen, to form the compound of formula (IV) below:

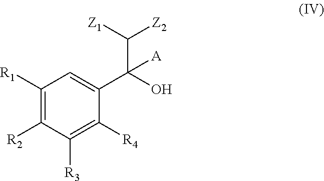

in which R₁, R₂, R₃, and R₄, are such as defined previously, and Z₁ and Z₂ are such as defined above in the context of this first variant,
reaction of the compound of formula (IV) obtained in the preceding step with an acid to give the compound of formula (II).

These steps may be followed by possible additional conventional steps for modification of the A substituents.

“Alkaline metal” means sodium (Na), lithium (Li) or potassium (K), in particular.

“Alkaline earth metal” means calcium (Ca) or magnesium (Mg), in particular.

Advantageously, M represents the lithium atom or the MgX group in which X represents a halogen, preferably bromine or chlorine, and advantageously bromine.

The A-Li derivative will then be advantageously obtained by reaction of the A-Hal derivative, where Hal represents a halogen atom such as an iodine, bromine or chlorine atom, with an (C₁ to C₆ alkyl)-Li derivative such as tert-BuLi.

If the magnesium compound of formula A-MgX is not available commercially, it can be prepared by reaction of an A-Hal derivative such as defined above with magnesium.

Also advantageously, the acid used in this last step is para-toluenesulfonic acid (PTSA).

According to a second variant, compounds of formula (II), for which Z₁ and Z₂ each represent a halogen atom or Z₁ represents a hydrogen atom and Z₂ represents a radical chosen from the group consisting of a hydrogen atom, a C₁ to C₄ alkyl group, —CN or —CO₂R, with R representing a C₁ to C₄ alkyl, can be prepared from the compound of formula (V) below:

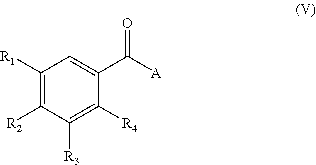

for which R₁, R₂, R₃, R₄ and A are such as defined previously,
by a Wittig reaction in the presence of a base and phosphonium of formula (VI) below

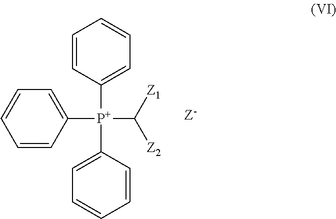

in which Z₁ and Z₂ are such as defined above in the context of the second variant and Z represents a bromine or chlorine atom,
this reaction may be followed by possible additional conventional steps for modification of the A substituents.

Advantageously, the base used for the Wittig reaction will be lithium hexamethyldisilazide (LiHMDS). THF can advantageously be used as the solvent.

In the case where Z₂ represents a —CO₂R₁₅ group with R₁₅ different from R such as defined above, and Z₁═H, the process described above for Z₂═CO₂R could be followed by an ester (CO₂R group) saponification step and a possible substitution step of the carboxylic acid thus obtained, so as to form the desired compound (II) for which Z₂═CO₂R₁₅.

Moreover, compounds of formula (II), for which Z₁ represents a hydrogen atom and Z₂ represents an —SO₃R₉ or SO₂NR₁₂R₁₃ group can be prepared according to the same process as the one described above in the second variant (process using a Wittig reaction), by replacing the previous phosphonium (VI) with a compound of general formula (VIbis) below:

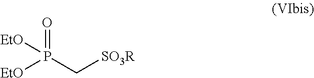

with R representing a C₁ to C₄ alkyl.

This Wittig reaction may possibly be followed by a step of saponification of the —SO₃R function to give —SO₃H, then a step of substitution or amidification of this —SO₃H function.

The base used in this case, for the Wittig reaction, will advantageously be n-butyl lithium.

The compound of formula (V) can be obtained notably by oxidation of the alcohol corresponding to formula (VII) below:

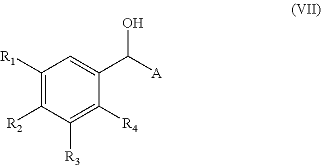

for which R₁, R₂, R₃, R₄ and A are such as defined previously,
by using, for example, manganese oxide or pyridinium chlorochromate (PCC).

The alcohol (VII) itself can be obtained from the aldehyde of formula (VIII) below:

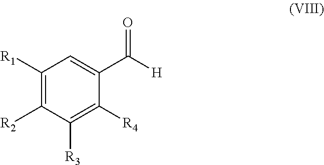

for which R₁, R₂, R₃ and R₄ are such as defined previously,
by reaction with an organometallic compound of formula A-M in which A and M are such as defined previously.

According to a third variant, compounds of formula (II), for which Z₁═H and Z₂ represents a hydrogen atom, a C₁ to C₄ alkyl or aryl group, can be prepared from the compound of formula (XI) below:

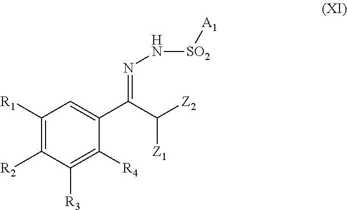

in which R₁, R₂, R₃, and R₄ are such as defined previously, and Z₁ and Z₂ are such as defined in the context of this third variant, and A₁ represents a phenyl group optionally substituted with one or more groups chosen from among C₁ to C₄ alkyl, such as methyl, C₁ to C₄ alkoxy, such as methoxy, and preferably represents a para-methyl-phenyl group.
by reaction with A-Z₃, with A such as defined above and Z₃ representing an halogen atom such as a bromine atom or an —OSO₂CF₃ group, in the presence of a catalyst and a base.

The base will advantageously be a lithiated base such as t-BuOLi.

The catalyst will advantageously be a palladium catalyst such as Pd₂ dba₃ used in association with a phosphine such as X-Phos.

The compound of formula (XI) can be prepared from the ketone of formula (XII) below:

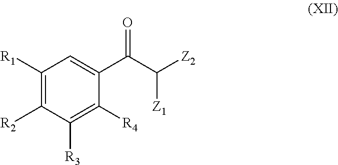

for which R₁, R₂, R₃, and R₄ are such as defined previously, and Z₁ and Z₂ are such as defined in the context of this third variant,
by reaction with para-toluenesulfonyl hydrazine.

According to a fourth variant, compounds of formula (II), for which Z₁═H and Z₂ represents a CO₂R₁₅ group, can be prepared from the compound of formula (XIII) below:

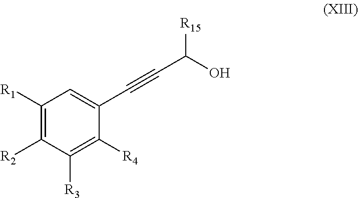

for which R₁, R₂, R₃, R₄ and R₁₅ are such as defined previously,
by a partial reduction of the triple bond, notably in the presence of LiAlH₄, to give the compound of formula (XIV) below:

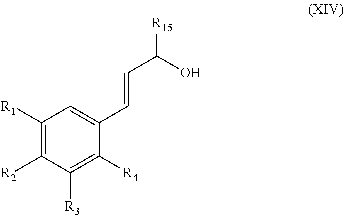

for which R₁, R₂, R₃, R₄ and R₁₅ are such as defined previously,
followed by an oxidation reaction to give the compound of formula (XV) below:

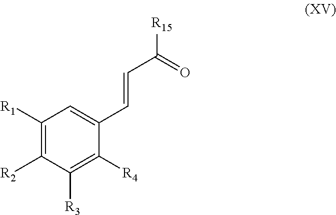

for which R₁, R₂, R₃, R₄ and R₁₅ are such as defined previously,
then finally a Heck reaction in the presence of A-Hal, with A such as defined above and Hal representing a halogen atom such as an iodide or bromine, to give the desired compound of formula (I) with Z₁═H and Z₂═CO₂R₁₅.

Furthermore, compounds of formula (I) in which X represents a nitrogen atom can be prepared according to the following successive steps:
reaction of a compound of formula (IX) below:

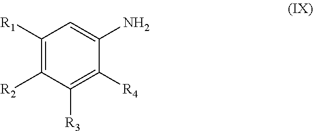

in which R₁, R₂, R₃, and R₄ are such as defined previously,
with a compound of formula A-Z₃, in which A and Z₃ are such as defined previously and in the presence of a catalyst and a base B1.
to give a compound of formula (X) below:

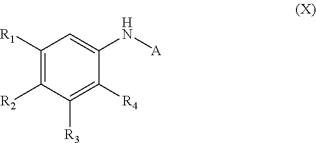

in which R₁, R₂, R₃, R₄ and A are such as defined previously,
reaction of the compound of formula (X) obtained from the preceding step with a compound of formula Z₁Z₂CH—X1, in which Z₁ and Z₂ are such as defined for the compound of formula (I) and X1 represents a halogen atom, advantageously an iodine or chlorine, in the presence of a base B2 to give the compound of formula (I) and
separation of the reaction medium from compound (I) obtained in the preceding step.

These steps may be followed by possible additional conventional steps for modification of the A groups and possibly the Z₂ group.

The compound thus obtained can be separated from the reaction medium by methods well-known to the person skilled in the art, such as, for example, by extraction, evaporation of the solvent or even by precipitation and filtration.

The compound can also be purified, if necessary, by techniques well known to the person skilled in the art, such as recrystallization if the compound is crystalline, by distillation, by silica gel column chromatography or even by high performance liquid chromatography (HPLC).

The base B1 will advantageously be cesium carbonate (Cs₂CO₃).

The catalyst will advantageously be a palladium catalyst such as Pd(OAc)₂ and will advantageously be used in the presence of a phosphine such as bis[-2-diphenylphosphinophenyl]ether (DPEphos) or 4,5-bis-(diphenylphosphino)-9,9-dimethylxanthene (XantPhos).

Advantageously, the base B2 will be sodium hydride and the alkylation reaction of the amine will advantageously be carried out at room temperature, notably in a solvent such as DMF.

Advantageously, Z₁ represents a hydrogen atom.

More advantageously, Z₂ represents a hydrogen atom, a C₁ to C₄ alkyl, an aryl or a —COR₁₅ group, and even more advantageously, represent a hydrogen atom or an acetyl.

The synthesis steps are thus compatible with industrial requirements. Furthermore, the analogs thus prepared having a sugar residue or a phosphate or boronic acid function are soluble in water and potentially can be assimilated orally.

The invention also has for a subject-matter, compounds of formula (I) as well as pharmaceutically acceptable salt thereof, isomers thereof and prodrugs thereof, for use thereof as medicaments, advantageously as medicaments inhibiting tubulin polymerization, and still more advantageously, as medicaments intended to treat or prevent proliferative diseases, such as cancer, psoriasis or fibrosis, and in particular cancer.

In particular, the compounds of the invention, including compounds of the formula:

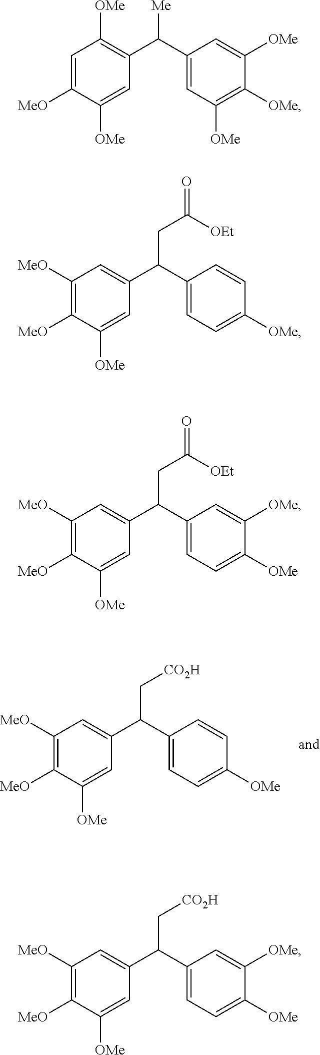

can be used in the treatment of cancer, such as those that can be treated by CA-4 or taxotere.

The invention also concerns the use of a compound of formula (I) or a compound of formula:

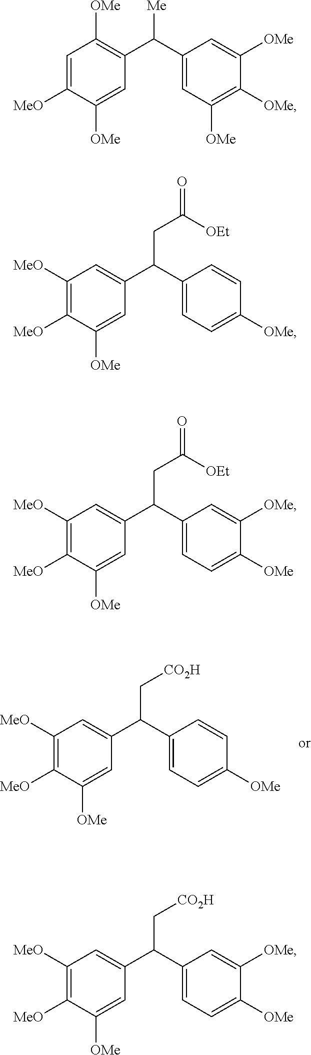

or one of pharmaceutically acceptable salts thereof, isomers thereof or prodrugs thereof, for the preparation of a medicament inhibiting tubulin polymerization, and advantageously intended to treat or prevent proliferative diseases, such as cancer, psoriasis or fibrosis, and in particular cancer.

The invention also has for a subject matter a pharmaceutical composition containing at least one a compound of formula (I) or a compound of formula:

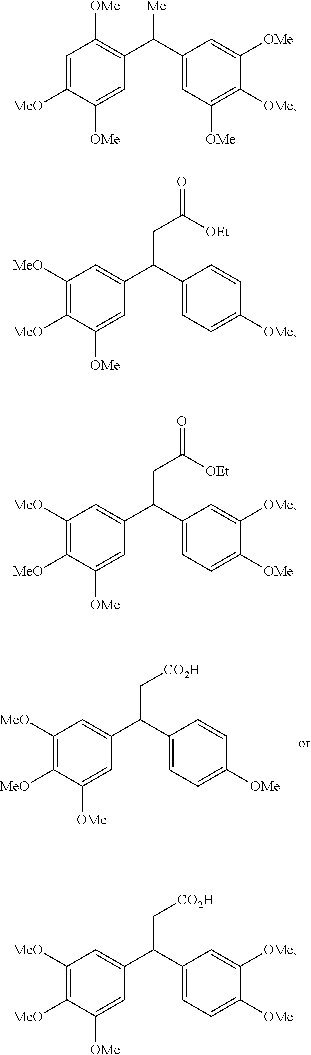

or one of pharmaceutically acceptable salts thereof, isomers thereof or prodrugs thereof, in combination with one or more pharmaceutically acceptable excipients.

The invention also has for a subject matter a pharmaceutical composition containing at least one compound of formula (I) or a compound of formula:

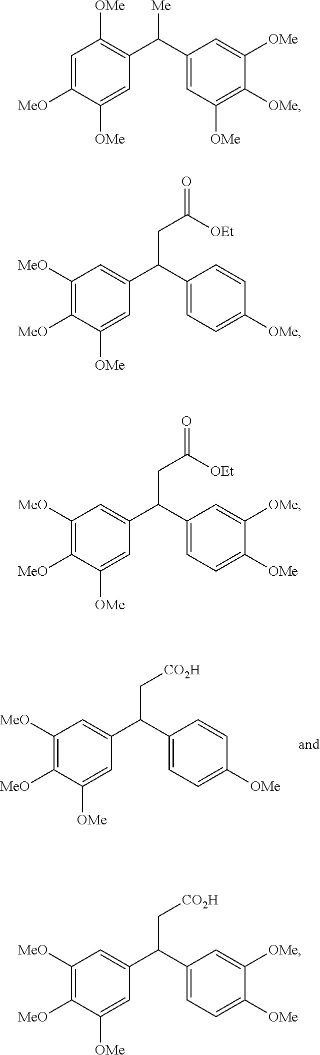

or one of pharmaceutically acceptable salts thereof, isomers thereof or prodrugs thereof, in combination with at least one active principle, notably an anti-cancer compound, possibly cytotoxic, in combination with one or more pharmaceutically-acceptable excipients.

As examples of active principles that can be combined with the compound of formula (I) in a composition according to the invention, we can name, in a non-limiting manner, 6-mercaptopurine, fludarabine, cladribine, pentostatin, cytarabine, 5-fluorouracil, gemcitabine, methotrexate, raltitrexed, irinotecan, topotecan, etoposide, daunorubicin, doxorubicin, epirubicin, idarubicin, pirarubicin, mitoxantrone, chlormethine, cyclophosphamide, ifosfamide, melphalan, chlorambucil, busulfan, carmustine, fotemustine, streptozocin, carboplatin, cisplatin, oxaliplatin, procarbazine, dacarbazine, bleomycin, vinblastine, vincristine, vindesine, vinorelbine, paclitaxel, docetaxel, L-asparaginase, flutamide, nilutamide, bicalutamide, cyproterone acetate, triptorelin, leuprorelin, goserelin, buserelin, formestane, aminoglutethimide, anastrazole, letrozole, tamoxifen, octreotide, lanreotide, (Z)-3-[2,4-dimethyl-5-(2-oxo-1,2-dihydro-indol-3-ylidenemethyl)-1H-pyrrol-3-yl]-propionic acid, 4-((9-chloro-7-(2,6-difluorophenyl)-5H-pyrimidol(5,4-d)(2)benzazepin-2-yl)amino)benzoic acid, 5,6-dimethylxanthenone-4-acetic acid or even 3-(4-(1,2-diphenylbut-1-enyl)phenyl)acrylic acid.

The compounds according to the invention can be administered orally, sublingually, parenterally, subcutaneously, intramuscularly, intravenously, transdermally, locally or rectally.

The compounds according to the invention can be used in the treatment and prevention of proliferative diseases such as cancers, psoriasis and fibrosis.

The can be used in doses comprised between 0.01 mg and 1000 mg per day, given in a single dose once a day or, preferably, administered in several doses throughout the day, for example twice a day in equal doses. The dose administered per day is advantageously comprised between 5 mg and 500 mg, and still more advantageously between 10 mg and 200 mg. It may be necessary to use doses exceeding these ranges, which the person skilled in the art can realize himself.

Compounds according to the invention can be used to decrease or inhibit tubulin polymerization, notably in vitro and also in vivo.

The present invention also has for a subject matter a pharmaceutical composition comprising:

(i) at least one compound of formula (I) or a compound of formula:

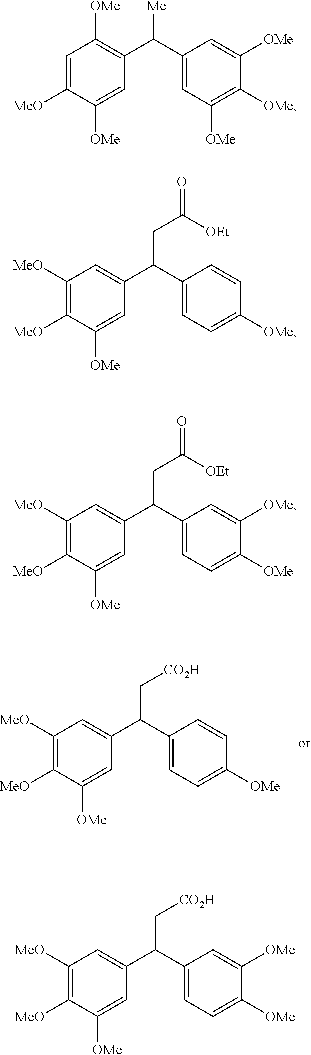

(ii) at least one other active principle, notably useful for the treatment of proliferative disorders such as cancer, psoriasis or fibrosis, and advantageously an anticancer agent such as an antivascular, cytotoxic or antiangiogenic agent, as combination products for simultaneous, separate or sequential use.

As an active principle, we can name, in a non-limiting manner, 6-mercaptopurine, fludarabine, cladribine, pentostatin, cytarabine, 5-fluorouracil, gemcitabine, methotrexate, raltitrexed, irinotecan, topotecan, etoposide, daunorubicin, doxorubicin, epirubicin, idarubicin, pirarubicin, mitoxantrone, chlormethine, cyclophosphamide, ifosfamide, melphalan, chlorambucil, busulfan, carmustine, fotemustine, streptozocin, carboplatin, cisplatin, oxaliplatin, procarbazine, dacarbazine, bleomycin, vinblastine, vincristine, vindesine, vinorelbine, paclitaxel, docetaxel, L-asparaginase, flutamide, nilutamide, bicalutamide, cyproterone acetate, triptorelin, leuprorelin, goserelin, buserelin, formestane, aminoglutethimide, anastrazole, letrozole, tamoxifen, octreotide, lanreotide, (Z)-3-[2,4-dimethyl-5-(2-oxo-1,2-dihydro-indol-3-ylidenemethyl)-1H-pyrrol-3-yl]-propionic acid, 4-((9-chloro-7-(2,6-difluorophenyl)-5H-pyrimidol(5,4-d)(2)benzazepin-2-yl)amino)benzoic acid, 5,6-dimethylxanthenone-4-acetic acid or even 3-(4-(1,2-diphenylbut-1-enyl)phenyl)acrylic acid.

The pharmaceutical composition such as described above can be useful, in particular, for the treatment of proliferative diseases, such as cancer, psoriasis or fibrosis, and in particular cancer.

The present invention also concerns the use of a pharmaceutical composition containing:

(i) at least one compound of formula (I) or a compound of formula:

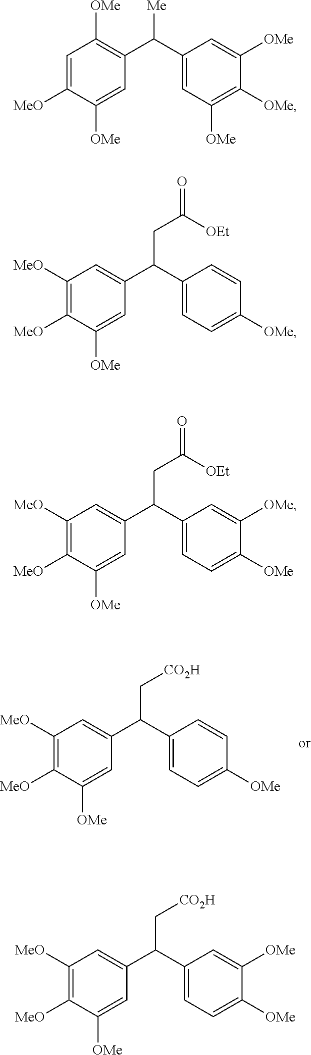

(ii) at least one other active principle, notably useful for the treatment of proliferative disorders such as cancer, psoriasis or fibrosis, and advantageously an anticancer agent such as an antivascular, cytotoxic or antiangiogenic agent,

as combination products for simultaneous, separate or sequential use, for the preparation of a medicament intended to treat proliferative diseases, such as cancer, psoriasis or fibrosis, in particular, cancer.

The invention will now be illustrated, in a non-limiting manner, by Examples 1 to 4 and FIGS. 1 to 4 that follow.

### FIGURES

FIG. 1 illustrates the cytotoxic activity of compound (I-1) on human endothelial cells EAhy926, measured immediately at the end of a treatment of 3 hours or 6 hours with compound (I-1).

FIG. 2 illustrates the cytotoxic activity of compound (I-1) on human endothelial cells EAhy926, measured after 72 hours of a treatment of 3, 6 or 72 hours.

FIG. 3 illustrates the antivascular activity of compounds (I-1) and (I-16), in comparison with 0.1% DMSO on human endothelial cells EAhy926, immediately after culture in matrigel.

FIG. 4 illustrates the antivascular activity of compounds (I-1) and (I-16), in comparison with 0.1% DMSO on human endothelial cells EAhy926 after 24 hours of culture in matrigel, in order to allow vascular tubes to form.

### EXAMPLES

### Example 1

### Synthesis of Molecules of the Invention

The following abbreviations are used:
APCI Atmospheric pressure chemical ionization
PTSA Para-toluenesulfonic acid
TLC Thin layer chromatography
dba Dibenzylideneacetone
DMAP Dimethylaminopyridine
DME 1,2-Dimethoxyethane
DMSO Dimethylsulfoxide
EDCI 1-Ethyl-3-(3-dimethylaminopropyl) carbodiimide
ESI Electrospray ionization
Fmoc 9-Fluorenylmethoxycarbonyl
HMDS 1,1,1,3,3,3-Hexamethyldisilazane
HPLC High performance liquid chromatography
MM Molecular mass
NMP N-Methyl-2-pyrrolidinone
PCC Pyridinium chlorochromate
Rf Frontal ratio
NMR Magnetic nuclear resonance
RT Room Temperature
TBAF Tetrabutylammonium fluoride
THF Tetrahydrofuran
X-Phos 2-Dicyclohexylphosphino-2′,4′,6′-triisopropylbiphenyl
Xantphos 4,5-Bis(diphenylphosphino)-9,9-dimethylxanthene

1.1. Synthesis of Intermediate Compounds of Formula (II)

Compound of Formula (II-1)

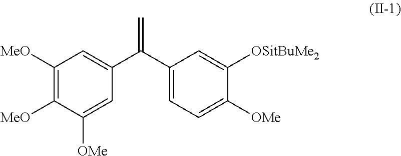

At 78° C., 1 mmol of tBuLi (2 eq.) is added to a solution containing 0.5 mmol of t-butyl[(5-iodo-2-methoxyphenoxy)]dimethylsilane dissolved in 15 ml of distilled hexane. After 45 minutes of stirring at this temperature, 0.5 mmol of 3,4,5-trimethoxyacetophenone diluted in 5 ml of distilled toluene are added. This mixture is stirred for 12 hours while letting the temperature progressively increase, then is slowly hydrolyzed by a saturated solution of NH₄Cl until pH=7-8. After extraction with diethyl ether (3×20 ml), the combined organic phases are dried on Na₂SO₄ and concentrated on a rotary evaporator. The crude reaction mixture is taken up in 10 ml of CH₂Cl₂ to which several grains of hydrated para-toluenesulfonic acid (PTSA) have been added then is stirred for 3 hours at room temperature. The solution is washed with a saturated solution of NaCl and extracted with CH₂Cl₂. After drying on Na₂SO₄ and concentration on a rotary evaporator, an oil is collected that is purified on silica gel. Yield 55%.

¹H NMR: δ, ppm, CDCl₃, 300 MHz: 0.14 (s, 6H), 0.97 (s, 9H), 3.81 (s, 6H), 3.82 (s, 3H), 3.87 (s, 3H), 5.30 (d, 1H, J=1.3 Hz), 5.35 (d, 1H, J=1.3 Hz), 6.54 (s, 2H), 6.80 (d, 1H, J=8.3 Hz), 6.85 (d, 1H, J=2.2 Hz), 6.91 (dd, 1H, J=8.3 Hz, J=2.2 Hz). Elementary analyses: (MM=430.22) Calculated C, 66.94; H, 7.96. Found C, 66.85; H, 7.92.

Compound of Formula (II-2)

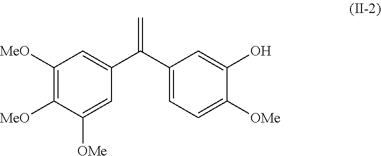

The silyl compound (II-1) (0.17 mmol) is dissolved in 10 ml of methanol to which 0.25 mmol of K₂CO₃ are added. The solution is stirred at room temperature for 12 hours then is washed with a saturated NaCl solution. The aqueous phase is extracted with ethyl acetate (3×10 ml). The combined organic phases are dried on Na₂SO₄ and concentrated on the rotary evaporator. The residue obtained is purified on silica gel. Yield 94%.

¹H NMR: δ, ppm, CDCl₃, 300 MHz: 3.81 (s, 6H), 3.87 (s, 3H), 3.91 (s, 3H), 5.30 (d, 1H, J=1.5 Hz), 5.37 (d, 1H, J=1.5 Hz), 5.60 (s, 1H), 6.55 (s, 2H), 6.82 (m, 2H), 6.97 (d, 1H, J=2.1 Hz). Mass spectrometry (ESI) [M+Na]⁺=339. Elementary analyses: (MM=316.13) Calculated C, 68.34; H, 6.37. Found C, 68.25; H, 6.33.

Compound of Formula (II-3)

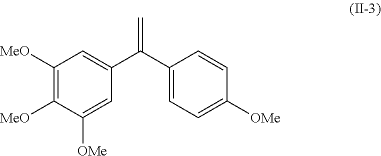

At 0° C. and under argon atmosphere, a commercial solution of (4-methoxyphenyl) magnesium bromide (2.2 mmol) is added slowly and dropwise to a solution containing 1 mmol of 3,4,5-trimethoxyacetophenone diluted in 5 ml of distilled tetrahydrofuran (THF). This mixture is stirred for 12 hours at room temperature then is hydrolyzed by addition of a saturated solution of NH₄Cl until pH=7-8. After extraction with dichloromethane (3×20 ml), the combined organic phases are dried on NA₂SO₄ and concentrated on a rotary evaporator. The crude reaction mixture is then treated like for (II-2) with para-toluenesulfonic acid to lead to the expected II-3 derivative after purification on silica gel. Yield 64%.

¹H NMR: δ, ppm, CD₃COCD₃, 300 MHz: 3.75 (s, 3H), 3.78 (s, 6H), 3.82 (s, 3H), 5.34 (m, 2H), 6.60 (s, 2H), 6.92 (d, 2H, J=8.7 Hz), 7.29, (d, 2H, J=8.7 Hz). Elementary analyses: (MM=300.14) Calculated C, 71.98; H, 6.71. Found C, 71.85; H, 6.66.

Compound of Formula (II-4)

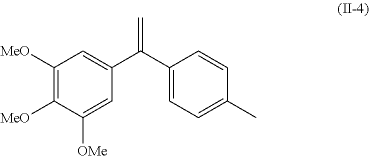

It was prepared according to the operating procedure described for the compound of formula (II-3) from para-tolylmagnesium bromide and 3,4,5-trimethoxyacetophenone. Yield 54%.

¹H NMR: δ, ppm, CDCl₃, 300 MHz: 2.33 (s, 3H), 3.75 (s, 3H), 3.76 (s, 6H), 5.38 (d, 1H, J=1.2 Hz), 5.40 (d, 1H, J=1.2 Hz), 6.59 (s, 2H), 7.22-7.25 (m, 4H). Elementary analyses: (MM=284.14) Calculated C, 76.03, H, 7.09. Found C, 75.74; H, 6.99.

Compound of Formula (II-5)

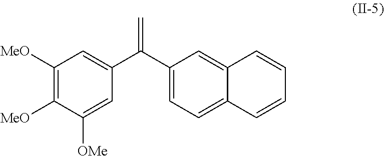

It was prepared from 2-naphtylmagnesium bromide and 3,4,5-trimethoxyacetophenone according to the operating procedure described for the compound of formula (II-3). Yield 81%.

¹H NMR: δ, ppm, CDCl₃, 300 MHz: 3.77 (s, 9H), 5.54-5.64 (m, 2H), 6.67 (s, 2H), 7.50-7.55 (m, 3H), 7.87-7.91 (m, 4H). Elementary analyses: (MM=320.14) Calculated C, 78.73; H, 6.29. Found C, 78.64; H, 6.20.

Compound of Formula (II-6)

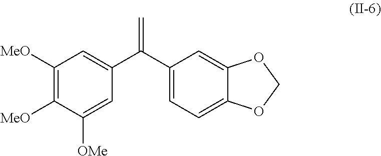

At 78° C., 1 mmol of tBuLi (2 eq.) is added to a solution containing 0.5 mmol of 5-bromo-benzo[1,3]dioxole dissolved in 15 ml of distilled hexane. After 45 minutes of stirring at this temperature, 0.5 mmol of 3,4,5-trimethoxyacetophenone diluted in 5 ml of distilled toluene are added. This mixture is stirred for 12 hours while letting the temperature progressively increase, then is slowly hydrolyzed with a saturated solution of NH₄Cl until pH=7-8. After extraction with diethyl ether (3×20 ml), the combined organic phases are dried on Na₂SO₄ and concentrated on a rotary evaporator. The crude reaction mixture is taken up in 10 ml of CH₂Cl₂ to which several grains of hydrated PTSA have been added, then is stirred for 3 hours at room temperature. The solution is washed with a saturated solution of NaCl and extracted with CH₂Cl₂. After drying on Na₂SO₄ and concentration on a rotary evaporator, an oil is collected that is purified on silica gel. Yield 19%.

¹H NMR: δ, ppm, CDCl₃, 300 MHz: 3.72 (s, 6H), 3.78 (s, 3H), 5.21 (d, 1H, J=1.5 Hz), 5.25 (d, 1H, J=1.5 Hz), 5.86 (s, 2H), 6.46 (s, 2H), 6.67 (d, 1H, J=8.7 Hz), 6.72-6.76 (m, 2H). Mass spectrometry (ESI) [M+Na]⁺=337. Elementary analyses: (MM=314.12) Calculated C, 68.78; H, 5.77. Found C, 68.68; H, 5.72.

Compound of Formula (II-7)

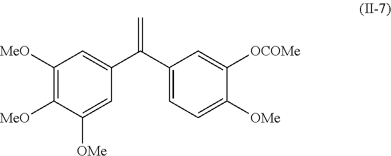

To a solution of compound (II-2) (0.316 mmol) dissolved in 1 ml of CH₂Cl₂ are added 54 μl of pyridine and 0.016 mmol of DMAP. The mixture is cooled to 0° C., then 42 μl of acetic anhydride (0.442 mmol) are added slowly. After 1 hour of stirring at 0° C., the reaction mixture is hydrolyzed (H₂O, 3 ml) then extracted with ethyl acetate (3×3 ml). The organic phases are collected, dried on sodium sulfate and concentrated to give a residue that is purified on silica gel. Yield 65%.

¹H NMR: δ, ppm, CDCl₃, 300 MHz: 2.28 (s, 3H), 3.74 (s, 6H), 3.78 (s, 3H), 3.84 (s, 3H), 5.26 (d, 1H, J=1.5 Hz), 5.31 (d, 1H, J=1.5 Hz), 6.48 (s, 2H), 6.86 (d, 1H, J=8.7 Hz), 6.97 (d, 1H, J=2.1 Hz), 7.16 (dd, 1H, J=8.4 Hz, J=2.1 Hz). Mass spectrometry (ESI) [M+Na]⁺=381. Elementary analyses: (MM=358.14) Calculated C, 67.03; H, 6.19. Found C, 66.88, H, 6.06.

Compound of Formula (II-8)

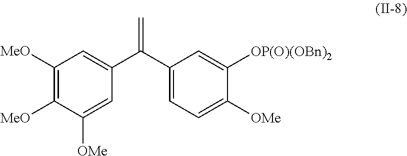

Compound (II-2) (0.136 mmol) is diluted in a mixture consisting of 153 μl of carbon tetrachloride and 1.3 ml of dry acetonitrile at −25° C. After 10 minutes of stirring, the following are successively added to the reaction mixture: diisopropylethylamine (0.663 mmol), dimethylaminopyridine (0.0136 mmol) and dibenzyl phosphite (0.458 mmol). After 1 hour 30 min of stirring at −25° C., the reaction mixture is hydrolyzed with an aqueous solution of KH₂PO₄ then extracted with ethyl acetate (3×3 ml). The organic phases are collected, dried on sodium sulfate and concentrated to give a residue that is purified on silica gel. Yield 40%.

¹H NMR: δ, ppm, CDCl₃, 300 MHz: 3.81 (s, 6H), 3.82 (s, 3H), 3.88 (s, 3H), 5.17 (d, 4H, J=7.8 Hz,), 5.32 (s, 1H), 5.33 (d, 1H, J=0.6 Hz), 6.55 (s, 2H), 6.89 (d, 1H, J=8.4 Hz), 7.14 (m, 1H), 7.23 (t, 1H, J=7.3 Hz), 7.23-7.4 (m, 10H). Mass spectrometry (ESI) [M+Na]⁺=599. Elementary analyses: (MM=576.19) Calculated C, 66.66; H, 5.77. Found C, 66.58; H, 5.72.

Compound of Formula (II-9)

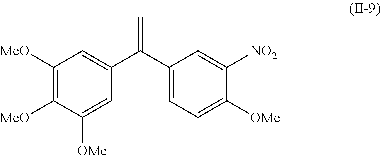

Under inert atmosphere, 1.07 g of methyl triphenylphosphonium bromide (3 mmol, 1 eq.) are diluted in 10 ml of THF. Then, 2.83 ml of a molar solution of lithium hexamethyldisilazide (LiHMDS) in THF (3 mmol) are added slowly and dropwise at 0° C. The reaction medium is stirred at 0° C. for 1 hour. The solution turns bright yellow. Then a solution of 520 mg of diarylketone (1.5 mmol) (prepared according to US 2005/107 339) in 10 ml of THF is added dropwise at 0° C. The mixture is stirred for 30 minutes under inert atmosphere at 0° C. and then at room temperature. 1 ml of water is added and then the medium is concentrated under vacuum. The residue is dissolved in 20 ml of dichloromethane then is washed 3 times with water. The organic phase is dried on MgSO₄ and then concentrated under vacuum. The residue obtained is chromatographed on silica gel. Yield 70%.

¹H NMR: δ, ppm, CDCl₃, 300 MHz: 3.82 (s, 6H), 3.88 (s, 3H), 3.98 (s, 3H), 5.44 (s, 2H), 6.80 (s, 2H), 7.05 (d, 1H, J=8.7 Hz), 7.52 (dd, 1H, J=8.7 Hz, J=2.3 Hz), 7.87 (d, 1H, J=2.3 Hz). Mass spectrometry (ESI)[M+Na]⁺=368.

Compound of Formula (II-10)

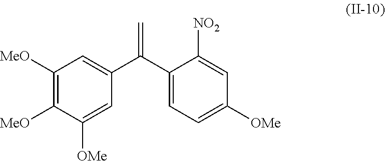

This compound was prepared according to the operating procedure described for the compound of formula (II-9) from the corresponding diarylketone (prepared according to US 2005/107 339). Yield 70%.

¹H NMR: δ, ppm, CDCl₃, 300 MHz: 3.77 (s, 6H), 3.83 (s, 3H), 3.89 (s, 3H), 6.44 (s, 2H), 7.14 (dd, 1H, J=8.4 Hz, J=2.7 Hz), 7.34 (d, 1H, J=8.4 Hz), 7.42 (d, 1H, J=2.7 Hz). Mass spectrometry (ESI) [M+Na]⁺=368.

Compound of Formula (II-11)

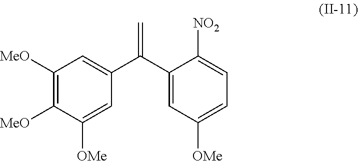

This compound was prepared according to the operating procedure described for the compound of formula (II-9) from the corresponding diarylketone (prepared according to US 2005/107 339). Yield 54%.

¹H NMR: δ, ppm, CDCl₃, 300 MHz: 3.77 (s, 6H), 3.83 (s, 3H), 3.92 (s, 3H), 6.45 (s, 2H), 6.91 (d, 1H, J=3.0 Hz), 6.96 (dd, 1H, J=9.0 Hz, J=3.0 Hz), 8.05 (d, 1H, J=9.0 Hz). Mass spectrometry (ESI) [M+Na]⁺=368.

Compound of Formula (II-12)

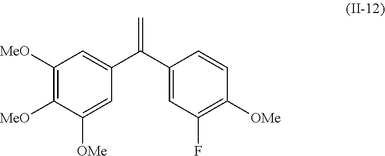

This compound was prepared according to the operating procedure described for the compound of formula (II-1) from 3,4,5-trimethoxyacetophenone and 2-fluoro-4-iodoanisole. Yield 48%.

¹H NMR: δ, ppm, CDCl₃, 300 MHz: 3.82 (s, 6H), 3.88 (s, 3H), 3.92 (s, 3H), 5.35 (d, 1H, J=1.5 Hz), 5.38 (d, 1H, J=1.5 Hz), 6.58 (s, 2H), 6.95 (m, 1H), 7.05-7.19 (m, 2H). Mass spectrometry (ESI)[M+Na]⁺=341.

Compound of Formula (II-13)

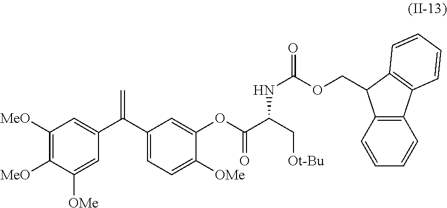

To a solution of compound (II-2) (0.79 mmol) in 15 ml of CH₂Cl₉ are added 0.94 mmol of 1-ethyl-3-(3-dimethylaminopropyl)carbodiimide hydrochloride (EDCl), 0.87 mmol of N,N-4-dimethylaminopyridine (DMAP) and 0.87 mmol of N-Fmoc serine (Ot-Bu) (serine whose amine function is protected by a 9-fluorenylmethoxycarbonyl (Fmoc) group and whose acid function is protected by a tert-butyl group). After stirring overnight, the reaction mixture is hydrolyzed with a saturated aqueous solution of NaHCO₃ then extracted with ethyl acetate (3×10 ml). The organic phases are collected, dried on sodium sulfate, filtered and the solvent is evaporated and then the residue obtained is chromatographed on silica gel. Yield 39%.

¹H NMR: δ, ppm, CDCl₃, 300 MHz: 1.09 (s, 9H), 3.64 (dd, 1H, J=9.0 Hz, J=2.7 Hz), 3.73 (s, 6H), 3.76 (s, 3H), 3.79 (s, 3H), 3.95 (dd, 1H, J=9.0 Hz, J=3.0 Hz), 4.19 (t, 1H, J=6.9 Hz), 4.27-4.39 (m, 2H), 4.72 (m, 1H), 5.26 (s, 1H), 5.31 (s, 1H), 6.67 (d, 1H, J=9.0 Hz), 6.47 (s, 2H), 6.87 (d, 1H, J=8.7 Hz), 6.98 (d, 1H, J=2.1 Hz), 7.18 (dd, 1H, J=8.4 Hz, J=2.4 Hz), 7.23 (d, 2H, J=7.5 Hz), 7.31 (t, 2H, J=7.2 Hz), 7.53 (m, 2H), 7.68 (d, 2H, J=7.2 Hz). Mass spectroscopy (ESI) [M+Na]⁺=704.

Compound of Formula (II-14)

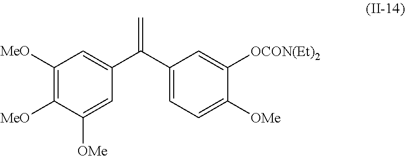

To a solution of compound (II-2) (0.316 mmol) in 2 ml of dry CH₂Cl₂ are added 54 μl of pyridine and 0.632 mmol of N,N-diethylcarbamic acid chloride. After stirring overnight at room temperature, the reaction mixture is hydrolyzed and extracted with ethyl acetate (3×3 ml). The organic phases are collected, dried on sodium sulfate, filtered and the solvent is evaporated. The residue obtained is chromatographed on silica gel. Yield 50%.

¹H NMR: δ, ppm, CDCl₃, 300 MHz: 1.11-1.20 (m, 6H), 3.28-3.39 (m, 4H), 3.75 (s, 6H), 3.77 (s, 3H), 3.80 (s, 3H), 5.25 (d, 1H, J=0.9 Hz), 5.32 (d, 1H, J=1.2 Hz), 6.50 (s, 2H), 6.82 (d, 1H, J=8.4 Hz), 7.05-7.10 (m, 2H). Mass spectroscopy (ESI) [M+Na]⁺=438.

Compound of Formula (II-15)

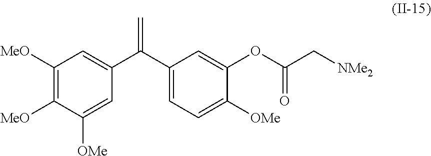

To a solution of compound (II-2) (0.316 mmol) in 5 ml of CH₂Cl₂ are added 0.47 mmol of EDCI, 0.47 mmol of DMAP and 0.47 mmol of N,N-dimethylglycine. After stirring overnight at room temperature, the reaction mixture is hydrolyzed with 6 ml of a saturated aqueous NaHCO₃ solution and extracted with ethyl acetate (3×3 ml). The organic phases are collected, dried on sodium sulfate, filtered and the solvent is evaporated. The residue obtained is chromatographed on silica gel. Yield 65%.

¹H NMR: δ, ppm, CDCl₃, 300 MHz: 2.37 (s, 6H); 3.37 (s, 2H); 3.74 (s, 6H), 3.77 (s, 3H), 3.80 (s, 3H), 5.26 (s, 1H), 5.31 (s, 1H), 6.47 (s, 2H), 6.86 (d, 1H, J=8.7 Hz), 6.97 (d, 1H, J=2.1 Hz), 7.16 (dd, 1H, J=8.4 Hz, J=2.1 Hz). Mass spectroscopy (ESI) [M+Na]⁺=424.

Compound of Formula (II-16)

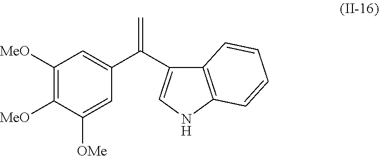

To an indole solution (165 mg, 1.41 mmol) in 5 ml of anhydrous THF, are added successively 1.83 mmol of 3,4,5-trimethoxyacetophenone and 0.14 mmol of TiCl₄. The mixture is stirred under nitrogen atmosphere at room temperature for 2 hours. 100 ml of water are added to the reaction mixture and a white suspension is formed that is filtered through sintered glass to deliver 150 mg of white powder. The filtrate is extracted with 3×30 ml of dichloromethane. The aqueous phase is then treated with a saturated solution of sodium carbonate to pH=10, then is extracted again with 3×30 ml of dichloromethane. The organic phase is washed with a saturated solution of sodium carbonate, then dried on sodium sulfate, filtered and concentrated under reduced pressure to provide 415 mg of crude product that is then dissolved in 5 ml of dichloromethane and 0.68 mmol of PTSA are added. The mixture is stirred under nitrogen atmosphere at room temperature for 30 minutes. 100 ml of a saturated solution of sodium carbonate are added, and the solution is extracted with 3×30 ml of dichloromethane. The organic phase is dried on sodium sulfate, filtered and concentrated under reduced pressure to provide 110 mg of crude product that is purified on silica column. Yield=70%.

¹H NMR: δ, ppm, CDCl₃, 300 MHz: 3.72 (s, 6H); 3.81 (s, 3H); 5.33 (s, 1H), 5.50 (s, 1H), 6.64 (s, 2H), 7.04-7.07 (m, 2H), 7.15 (t, 1H, J=8.0 Hz), 7.33 (d, 1H, J=8.0 Hz), 7.56 (d, 1H, J=8.0 Hz), 8.30 (sl, 1H). Mass spectroscopy (ESI) [M+Na]⁺=332.0.

Compound of Formula (II-17)

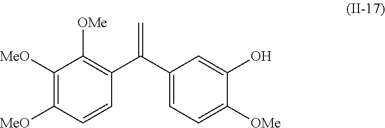

At −78° C., 1 mmol of tBuLi (2 eq.) is added to a solution containing 0.5 mmol of t-butyl[(5-iodo-2-methoxyphenoxy)]dimethylsilane dissolved in 15 ml of distilled hexane. After 45 minutes of stirring at this temperature, 0.5 mmol of 2,3,4,-trimethoxyacetophenone diluted in 5 ml of distilled toluene are added. This mixture is stirred for 12 hours while letting the temperature progressively increase, then is slowly hydrolyzed with a saturated solution of NH₄Cl with pH=7-8. After extraction with diethyl ether (3×20 ml), the combined organic phases are dried on Na₂SO₄ and concentrated on a rotary evaporator. The crude reaction mixture is dissolved in 10 ml of methanol to which 0.25 mmol of K₂CO₃ are added. The solution is stirred at room temperature for 12 hours then is washed with a saturated NaCl solution. The aqueous phase is extracted with ethyl acetate (3×10 ml). The combined organic phases are dried in Na₂SO₄ and concentrated on a rotary evaporator to give a residue that is then purified on silica gel. Yield 51%.

¹H NMR: δ, ppm, CDCl₃, 300 MHz: 3.60 (s, 3H), 3.82 (s, 3H), 3.84 (s, 3H), 3.88 (s, 3H), 5.21 (d, 1H, J=1.5 Hz), 5.54 (d, 1H, J=1.5 Hz), 5.63 (sl, 1H), 6.70 (d, 1H, J=8.7 Hz), 6.78-6.84 (m, 2H), 6.95-6.99 (m, 2H). Mass spectroscopy (ESI) [M+Na]⁺=339.

Compound of Formula (II-18)

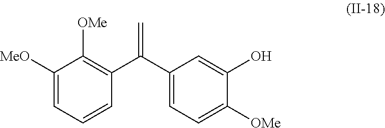

At −78° C., 1 mmol of tBuLi (2 eq.) is added to a solution containing 0.5 mmol of t-butyl[(5-iodo-2-methoxyphenoxy)]dimethylsilane dissolved in 15 ml of distilled hexane. After 45 minutes of stirring at this temperature, 0.5 mmol of 2,3-dimethoxybenzaldehyde diluted in 5 ml of distilled toluene are added. This mixture is stirred for 12 hours while letting the temperature progressively increase, then is slowly hydrolyzed with a saturated solution of NH₄Cl until pH=7-8. After extraction with diethyl ether (3×20 ml), the combined organic phases are dried on Na₂SO₄ and concentrated on a rotary evaporator. The crude reaction mixture is diluted in 30 ml of CH₂Cl₂ and 3 equivalents of PDC are added portionwise. This is all stirred for 12 h at room temperature and then filtered through silica. After concentration on a rotavapor, the crude ketone is obtained, sufficiently clean to be used without purification. Yield 87%. The crude ketone is treated according to the protocol described for (II-9) and is then desilylated without previous purification according to the protocol described for (II-2). Yield 54%.

¹H NMR: δ, ppm, CDCl₃, 300 MHz: 3.50 (s, 3H), 3.80 (s, 6H), 5.17 (d, 1H, J=1.5 Hz), 5.49 (s, 1H), 5.60 (d, 1H, J=1.5 Hz), 6.70-6.98 (m, 6H). Mass spectroscopy (ESI) [M+Na]⁺=309.

Compound of Formula (II-19)

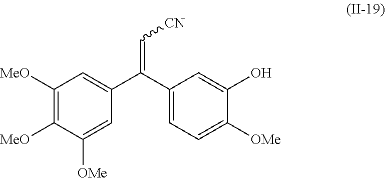

This compound (1/1 mixture of Z/E isomers) was prepared according to the operating procedure described for the compound of formula (II-9) from silylated phenstatin (G. R. Pettit et al. J. Med. Chem. 1998, 41, 1688-1695) and the corresponding ylide prepared from cyanomethyl triphenylphosphonium bromide. (Yield 87%).

¹H NMR: δ, ppm, CDCl₃, 300 MHz: 3.80 (s, 3H), 3.83 (s, 3H), 3.88 (s, 1.5H), 3.91 (s, 1.5H), 3.93 (s, 1.5H), 3.95 (s, 1,5H), 5.56 (s, 0.5H), 5.60 (s, 0.5H), 5.67 (s, 1H), 6.49 (s, 1H), 6.36 (s, 1H), 6.83 (s, 1H), 6.90-6.95 (m, 1.5H), 7.10 (dd, 0.5H, J=9.0 Hz, J=2.1 Hz). Mass spectroscopy (ESI) [M+Na]⁺=364.

Compound of Formula (II-20)

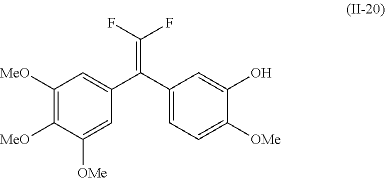

This compound was prepared according to the operating procedure described for the compound of formula (II-9) from silylated phenstatin (G. R. Pettit et al. J. Med. Chem. 1998, 41, 1688-1695) and the corresponding ylide prepared from ethyl difluoromethylphosphate. (Yield 89%).

¹H NMR: δ, ppm, CDCl₃, 300 MHz: 3.85 (s, 6H), 3.92 (s, 3H), 3.99 (s, 3H), 5.68 (s, 1H), 6.50 (s, 2H), 6.80-6.98 3H). Mass spectrometry (ESI) [M+Na]⁺=375.2.

Compound of Formula (II-21)

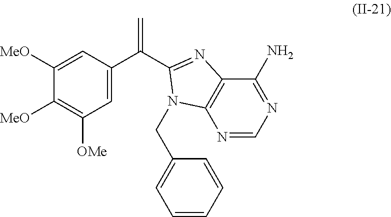

A solution of N-benzyladenine (1.0 mmol, 1 eq.) and 1-iodo-1-(3,4,5-trimethoxyphenyl)ethene (1.5 mmol, 1.5 eq.) in the presence of CsCO₃ (2.0 mmol, 2 eq.), CuI (2.0 mmol, 2 eq.) and Pd(OH)₂/C (20% by mass) is prepared in a dry tube, capped by a septum. After an argon flow, NMP (6 ml) is added through the septum by means of a syringe. After this operation, the tube is sealed, and the mixture is stirred at 160° C. under microwave irradiations for 30 minutes. The resulting suspension is cooled to room temperature and filtered through sintered glass bearing a thin layer of celite and using a mixture of CH₂Cl₂/MeOH (7:3, v/v) as the elution solvent. The filtrate is concentrated and the residue is purified by silica gel column chromatography (cyclohexane/ethyl acetate: 7:3. (Yield 40%).

¹H NMR: δ, ppm, CDCl₃, 300 MHz: 3.68 (s, 3H), 3.73 (s, 2H), 5.25 (s, 2H), 5.65 (s, 1H), 5.98 (s, 1H), 6.52 (s, 2H), 6.96-7.20 (m, 1H), 8.28 (bs, 1H). Mass spectroscopy (positive ESI): [M+H]⁺=418, [M+Na]⁺=440.

Compound of Formula (II-22)

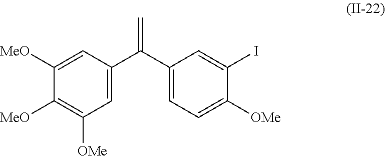

Step 1: Synthesis of Compound (V-I) Below

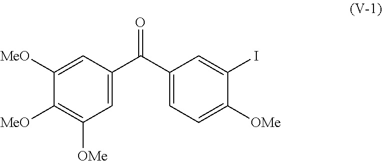

At 0° C. and under argon atmosphere, a 1M (2.2 ml) solution of 3,4,5-trimethoxyphenyl magnesium bromide

(2.2 mmol) is slowly added to a solution containing 1 mmol of 3-iodo-4-methoxybenzaldehyde diluted in 5 ml of distilled tetrahydrofuran (THF). This mixture is stirred for 12 hours at room temperature then is hydrolyzed by addition of a saturated solution of NH₄Cl until pH=7-8. After extraction with dichloromethane (3×20 ml), the combined organic phases are dried on Na₂SO₄ and concentrated on a rotary evaporator. The crude reaction secondary alcohol is then mixed with 215.5 mg of pyridinium chlorochromate (PCC, 1 eq.) in CH₂Cl₂ for 1 h. Then an additional 215 mg PCC are added to the reaction medium, which is stirred for 1 h at room temperature. This operation is repeated with 100 mg of PCC and the mixture is reacted for an additional 2 h. The crude reaction mixture is filtered through silica and then concentrated on a rotary evaporator. The residue obtained is chromatographed on silica gel. (Yield for the 2 steps: 40%).

¹H NMR: δ, ppm, CDCl₃, 300 MHz: 3.90 (s, 6H), 3.95 (s, 3H), 4.00 (s, 3H), 6.90 (d, 1H, J=8.5 Hz), 6.95 (s, 2H), 7.82 (dd, 1H, J=8.5 Hz, J=1.2 Hz), 8.32 (d, 1H, J=1.2 Hz). Elementary analyses: (MM=428.22) Calculated C, 47.68; H, 4.00. Found C, 47.48; H, 3.92.

Step 2: Synthesis of Compound (II-22)

Under inert atmosphere, 1.07 g of methyl triphenylphosphonium bromide (3 mmol, 1 eq.) are diluted in 10 ml of THF. Then, 2.83 ml of a molar solution of lithium hexamethyldisilazide (LiHMDS) in THF (3 mmol) are added slowly and dropwise at 0° C. The reaction medium is stirred at 0° C. for 1 hour. The solution turns bright yellow. Then a solution of 520 mg of compound (V-1) (1.5 mmol) in 10 ml of THF is added dropwise at 0° C. The mixture is stirred for 30 minutes under inert atmosphere at 0° C. and then at room temperature. 1 ml of water is added to the medium and then the medium is concentrated under vacuum. The residue is dissolved in 20 ml of dichloromethane then is washed 3 times with water. The organic phase is dried on MgSO₄ and then condensed under vacuum. The residue obtained is chromatographed on silica gel. (Yield 82%).

¹H NMR: δ, ppm, CDCl₃, 300 MHz: 4.07 (s, 6H), 4.13 (s, 3H), 4.15 (s, 3H), 5 60 (d, 2H, J=5.0 Hz), 6.70 (s, 2H), 7.03 (d, 1H, J=8.5 Hz), 7.53 (dd, 1H, J=8.5 Hz, J=1.2 Hz), 8.06 (d, 1H, J=1.2 Hz). Mass spectroscopy (APCI+) [M+H]⁺=427.

Compound of Formula (II-23)

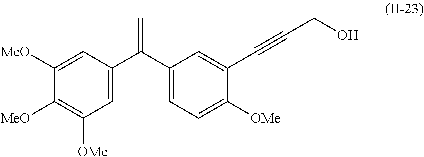

1.2 ml of distilled triethylamine, 100 mg of PdCl₂(PPh₃)₂, and 54 mg of cuprous iodide are added to a solution of compound II-22 (1.1 g, 2.58 mmol, 1 eq.) in 25 ml of tetrahydrofuran, under inert atmosphere. After the reaction mixture is stirred, it is immersed into an oil bath at 60° C. and a solution of prop-2-yn-1-ol (0.5 ml, 3.3 eq.) in 25 ml of THF, is added dropwise. After 16 hours of stirring at 60° C. under inert atmosphere, the reaction medium is taken up with 40 ml ethyl acetate. The organic phase is washed with a saturated solution of NH₄Cl (3×30 ml), dried on sodium sulfate, filtered through sintered glass, then concentrated under vacuum. The residue is then purified by silica gel column chromatography. (Yield 50%).

¹H NMR: δ, ppm, CDCl₃, 300 MHz: 3.75 (s, 6H), 3.81 (s, 3H), 3.84 (s, 3H), 4.46 (s, 2H), 5.28 (d, 2H, J=8.5 Hz), 6.45 (s, 2H), 6.78 (d, 1H, J=8.5 Hz), 7.24 (m, 1H₅), 7.37 (d, 1H, J=2.3 Hz). Mass spectroscopy (APCI+) [M+H]⁺=355.

Compound of Formula (II-24)

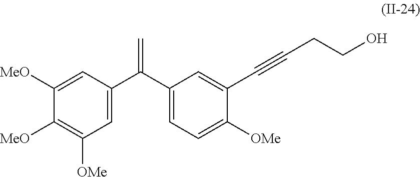

Compound (II-24) is prepared according to the operating protocol described for (II-23) by using 3.3 equivalents of but-3-yn-1-ol and after 16 h of stirring. (Yield 46%).

¹H NMR: δ, ppm, CDCl₃, 300 MHz: 2.42 (t, 2H, J=6.3 Hz), 2.64 (t, 2H, J=6.3 Hz), 3.73 (s, 6H), 3.79 (s, 3H), 3.81 (s, 3H), 5.26 (d, 2H, J=6.7 Hz), 6.44 (s, 2H), 6.75 (d, 1H₆, J=8.6 Hz), 7.15 (dd, 1H, J=2.2 Hz, J=8.6 Hz), 7.32 (d, 1H, J=2.2 Hz). Mass spectrometry (APCI+) [M+H]⁺=369.

Compound of Formula (II-25)

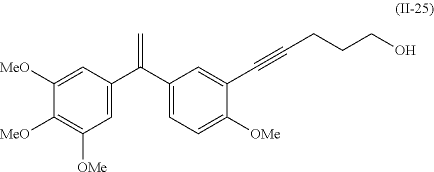

Compound (II-25) is prepared according to the operating protocol described for (II-23) by using 3.3 equivalents of pent-4-yn-1-ol and after 16 h of stirring.

Yield 54%.

¹H NMR: δ, ppm, CDCl₃, 300 MHz: 1.88 (q, 2H, J=6.8 Hz), 2.60 (t, 2H, J=6.8 Hz), 3.81 (m, 8H), 3.88 (s, 3H), 3.89 (s, 3H), 5.34 (d, 2H, J=7.2 Hz), 6.52 (s, 2H), 6.81 (d, 1H, J=8.6 Hz), 7.22 (dd, 1H, J=2.3 Hz, J=8.6 Hz), 7.39 (d, 1H, J=2.3 Hz). Mass spectrometry (APCI+) [M+H]⁺=383.

Compound of Formula (II-26)

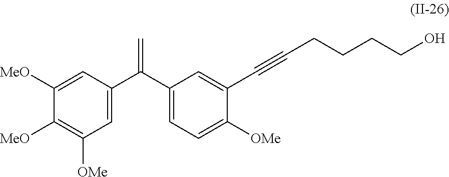

Compound (II-26) is prepared according to the operating protocol described for (II-23) by using 3.3 equivalents of hex-5-yn-1-ol and after 16 h of stirring. (Yield 45%).

¹H NMR: δ, ppm, CDCl₃, 300 MHz: 1.73 (m, 4H), 2.51 (t, 2H, J=6.5 Hz), 3.71 (t, 2H, J=5.5 Hz), 3.80 (s, 6H), 3.87 (s, 3H), 3.89 (s, 3H), 5.34 (d, 2H, J=7.9 Hz), 6.52 (s, 2H), 6.81 (d, 1H, J=8.6 Hz), 7.20 (dd, 1H, J=2.2 Hz, J=8.6 Hz), 7.39 (d, 1H, J=2.2 Hz). Mass spectrometry (APCI+) [M+H]⁺=397.

Compound of Formula (II-27)

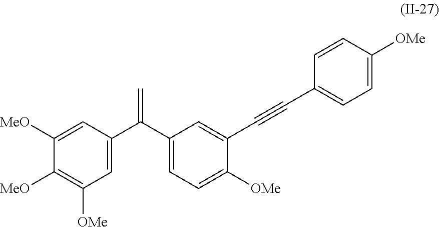

Compound (II-27) is prepared according to the operating protocol described for (II-23) by using 2.0 equivalents of 4-methoxyphenyl-1-ethyne and after 16 h of stirring. (Yield 80%).

¹H NMR: δ, ppm, CDCl₃, 300 MHz: 3.78 (s, 3H), 3.81 (s, 6H), 3.88 (s, 3H), 3.91 (s, 3H), 5.37 (d, 2H, J=10.6 Hz), 6.55 (s, 2H), 6.84 (m, 3H), 7.25 (dd, 1H, J=2.0 Hz, J=8.8 Hz), 7.52-7.46 (m, 3H). Mass spectrometry (APCI+) [M+H]⁺=431.

Compound of Formula (II-28)

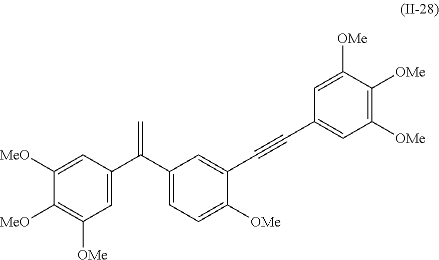

Compound (II-28) is prepared according to the operating protocol described for (II-23) by using 2.5 equivalents of 3,4,5-trimethoxyphenyl-1-ethyne and after 16 h of stirring. (Yield 74%).

¹H NMR: δ, ppm, CDCl₃, 300 MHz: 3.78 (s, 3H), 3.81 (s, 6H), 3.88 (s, 3H), 3.91 (s, 3H), 5.37 (d, 2H, J=10.6 Hz), 6.55 (s, 2H), 7.25 (s, 2H), 7.30 (d, 1H, J=8.7 Hz), 7.48 (dd, 1H, J=2.0 Hz, J=8.7 Hz), 7.51 (d, 1H, J=2.0 Hz). Mass spectrometry (APCI+) [M+H]⁺=491.

Compound of Formula (II-29)

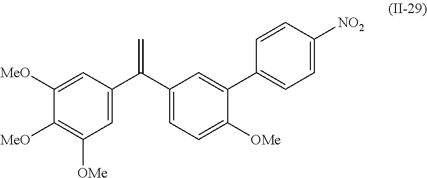

A solution of (II-22) (426 mg; 1 mmol; 1 eq.) in 2 ml of DME are added under inert atmosphere at room temperature to 4-nitrophenyl boronic acid (488 mg, 2.5 mmol), NaHCO₃ (420 mg, 5 eq.) in 0.4 ml of distilled water and Pd(PPh₃)₄ (70 mg, 0.06 mmol) The mixture is brought to reflux for 24 hours. The organic phase is washed with a saturated solution of NH₄Cl (3×30 ml), dried on sodium sulfate, filtered through sintered glass, then concentrated under vacuum. The residue is then purified by silica gel column chromatography. (Yield 56%).

¹H NMR: δ, ppm, CDCl₃, 300 MHz: 3.76 (s, 6H), 3.80 (s, 3H), 3.81 (s, 3H), 5.32 (d, 2H, J=10.6 Hz), 6.51 (s, 2H), 6.91 (d, 1H, J=8.3 Hz), 7.19 (s, 1H), 7.29 (m, 1H), 7.62 (d, 2H, H₁₆, J=8.0 Hz), 8.18 (d, 2H, J=8.0 Hz). Mass spectrometry (APCI+)[M+H]⁺=422

Compound of Formula (II-30)

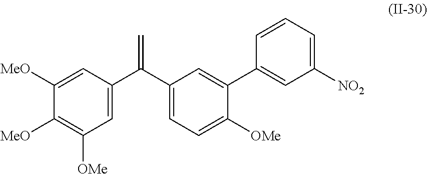

Compound (II-30) is prepared following the operating protocol described for (II-29) using 3-nitrophenyl boric acid. (Yield 60%).

¹H NMR: δ, ppm, CDCl₃, 300 MHz: 3.83 (s, 6H), 3.87 (s, 3H), 3.88 (s, 3H), 5.41 (d, 2H, J=11.8 Hz), 6.59 (s, 2H), 6.99 (d, 1H, J=8.1 Hz), 7.36 (m, 2H), 7.56 (t, 1H J=7.9 Hz,), 7.82-7.86 (m, 1H), 8.15-8.19 (m, 1H), 8.41 (t, 1H, J=1.9 Hz). Mass spectrometry (APCI+) [M+H]⁺=422

Compound of Formula (II-31)

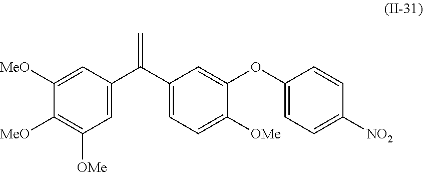

To a suspension of K₂CO₃ (1 eq.) in anhydrous DMSO (5 ml), II-2 (1 eq.) is added at −15° C. After stirring for 30 minutes at room temperature, a solution of 4-fluoro-nitrobenzene (1 eq.) is added dropwise and the reaction tube is sealed. After stirring for 1 h at 100° C., the crude reaction mixture is extracted with ethyl acetate (10 ml) then washed with saturated NH₄Cl solution (10 ml). The crude mixture is then purified on a silica gel column. (Yield 99%).

¹H NMR: δ, ppm, CDCl₃, 300 MHz: 3.80 (s, 3H), 3.81 (s, 6H), 3.86 (s, 3H), 5.38 (d, 2H, J=13.8 Hz), 6.54 (s, 2H), 6.94 (d, 2H, J=9.3 Hz), 7.01 (d, 1H J=8.5 Hz), 7.13 (d, 1H, J=2.2 Hz), 7.29 (dd, 1H, J=2.2 Hz, J=8.5 Hz), 8.16 (d, 2H, J=9.3 Hz). Mass spectrometry (APCI+) [M+H]⁺=438

Compound of Formula (II-32)

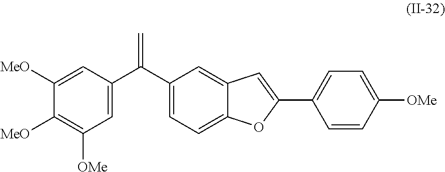

The derivative (II-27) (100 mg, 1 eq.) and para-toluene sulphonic acid (PTSA, 0.1 eq.) are placed in solution in 3 ml of ethanol in a sealed tube (M. Jacubert et al, Tetrahedron Lett. 2009, 50, 3588-3592). This tube is heated to 170° C. under microwave radiations for 30 minutes. After adding ethyl acetate to the reaction medium (3 ml), the organic phase is washed with water, dried over sodium sulphate, filtered then concentrated. The residue is purified by chromatography on silica gel column (Yield 71%).

¹H NMR: δ, ppm, CDCl₃, 300 MHz: 3.81 (s, 6H), 3.86 (s, 3H), 3.90 (s, 3H), 5.44 (dd, 2H, J=1.2 Hz, J=6.8 Hz), 6.60 (s, 2H), 6.86 (s, 1H), 6.98 (d, 2H, J=8.8 Hz), 7.27 (dd, 1H, J=1.8 Hz, J=8.5 Hz), 7.5 (d, 1H, J=8.5 Hz), 7.54 (bs, 1H, J=1.8 Hz), 7.80 (d, 2H, J=8.8 Hz). Mass spectrometry (APCI+) [M+H]⁺=417.

Compound of Formula (II-33)

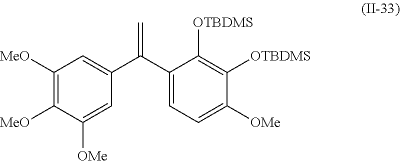

### Step 1: Synthesis of Compound (XI-I)

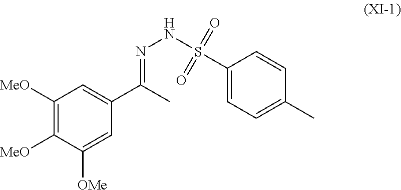

5 g of 3,4,5-trimethoxyacetophenone (24 mmol; 1 eq.) and 5.48 g of p-toluenesulfonyl hydrazine (28.8 mmol; 1.2 eq.) are placed in solution in 100 ml of absolute ethanol. The reaction medium (yellow solution) is stirred under reflux and the reaction is followed by TLC (Cyclohexane/ethyl acetate: 7:3, Rf=0.49). After 4 h, the reaction medium (yellow solution) is cooled to 0° C. A yellow precipitate is formed. This precipitate is collected by filtering through a sintered filter and washed with cold ethanol, then recrystallized in ethanol (Yield 79%).

¹H NMR: δ, ppm, CDCl₃, 300 MHz: 2.14 (s, 3H), 2.41 (s, 3H), 3.85 (s, 3H), 3.86 (s, 6H), 6.86 (s, 2H), 7.31 (d, 2H J=805 Hz), 7.74 (s, 1H), 7.92 (d, 2H, J=8.20 Hz). Mass spectrometry (APCI+) [M+H]⁺=379.

### Step 2: Synthesis of Compound (II-33)

To a solution of 454 mg of XI-I (1.2 mmol; 1.2 eq.), 196 mg of t-BuOLi (2.4 mmol; 2.4 eq.), 52 mg of Pd₂ dba₃ (0.005 mmol; 10%), 98 mg of X-Phos in 20 ml dioxane is added a solution of 448 mg of 1-bromo-2,3-di-tert-butyldimethylsilyloxy-4-methoxybenzene (1 mmol; 1 eq.) in 5 ml dioxane at room temperature. The reaction medium is then heated to 70° C. and the reaction is followed by TLC (Cyclohexane/ethyl acetate—7:3 for hydrazine and cyclohexane for the aromatic bromine derivative). After 6 h, the reaction medium is cooled to room temperature and diluted with CH₂Cl₂ then filtered through celite and concentrated under reduced pressure. The crude product is purified on silica gel column (Cyclohexane/ethyl acetate—9:1). (Yield 82%).

¹H NMR: δ, ppm, CDCl₃, 300 MHz: 0.05 (s, 6H), 0.71 (s, 6H), 0.76 (s, 9H), 0.98 (s, 9H), 3.77 (s, 6H), 3.79 (s, 3H), 3.83 (s, 3H), 5.32 (s, 1H), 5.65 (s, 1H), 6.52 (d, 1H, J=8.4 Hz), 6.56 (s, 2H), 6.78 (d, 1H, J=8.5 Hz).

Compound of Formula (II-34)

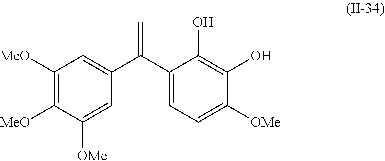

To a solution of 0.46 g of compound II-33 (8.2 mmol, 1 eq.) in 20 ml of THF are added 11.5 ml of 1M tetra-butyl ammonium fluoride (11.5 mmol, 1.4 eq.) at 0° C. The reaction medium is stirred at room temperature and followed by TLC (Cyclohexane/ethyl acetate-9:1). After 1.5 h0 the reaction medium is hydrolysed, the THF is evaporated and the residue taken up with ethyl acetate. The organic phases are washed with saturated NaCl solution, dried over MgSO₄, and concentrated under reduced pressure. The crude product is purified on a silica gel column (Cyclohexane/ethyl acetate-1:1). (Yield 37%).

¹H NMR: δ, ppm, CDCl₃, 300 MHz: 3.78 (s, 6H), 3.84 (s, 3H), 3.87 (s, 3H), 5.35 (d, 1H, J=1.3 Hz), 5.47 (s, 1H), 5.65 (d, 1H, J=1.3 Hz), 5.69 (s, 1H), 6.48 (d, 1H, J=8.6 Hz), 6.57 (s, 2H), 6.69 (d, 1H, J=8.5 Hz). Mass spectrometry (ESI, m/z,%): 355 (M+Na, 100).

Compound of Formula (II-35)

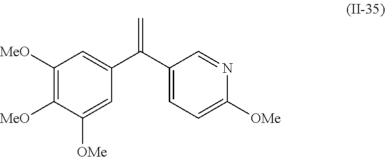

This compound was prepared following the operating mode described for the compound of formula (II-33) from 5-bromo-2-methoxypyridine. (Yield 60%).

¹H NMR: δ, ppm, CDCl₃, 300 MHz: 3.80 (s, 6H), 3.85 (s, 3H), 3.94 (s, 3H), 5.37 (d, 2H, J=1.2 Hz), 6.51 (s, 2H), 6.70 (d, 1H, J=8.6 Hz), 7.51 (dd, 1H, J=2.5 Hz, J=8.6 Hz), 8.17 (d, 1H, J=2.4 Hz). Mass spectrometry (APCI+) [M+H]⁺=302.

Compound of Formula (II-36)

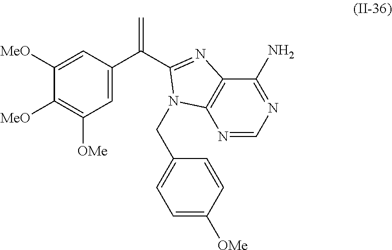

This compound was prepared following the operating mode described for the compound of formula (II-21) by coupling between 1-iodo-1-(3,4,5-tri-methoxyphenyl)ethene and N⁹-4-methoxybenzyladenine (Yield 42%).

¹H NMR: δ, ppm, CDCl₃, 300 MHz: 3.71 (s, 9H), 3.84 (s, 3H), 5.10 (s, 2H), 5.65 (s, 1 Hz), 5.90 (s, 2H), 6.25 (s, 2H), 6.34 (s, 2H), 6.68 (d, 2H, J=8.4 Hz), 6.93 (d, 2H, J=8.4 Hz), 8.40 (se, 1H). Mass spectrometry (APCI+) [M+H]+=448

Compound of Formula (II-37)

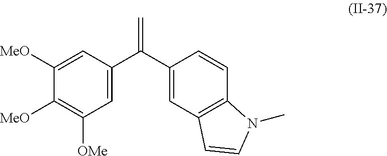

This compound was prepared following the operating mode described for formula (II-33) from 5-bromo-N-methylindole. (Yield 53%).

¹H NMR: δ, ppm, CDCl₃, 300 MHz: 3.79 (s, 6H), 3.83 (s, 3H), 3.84 (s, 3H), 5.38 (d, 1H, J=1.5 Hz)), 5.41 (d, 1H, J=1.5 Hz), 6.44 (dd, 1H, J=0.6 Hz, J=3.1 Hz), 6.66 (s, 2H), 7.18 (d, 1H, J=3.2 Hz), 7.18 (dd, 1H, J=1.7 Hz, J=8.3 Hz), 7.35 (d, 1H, J=8.5 Hz), 7.53 (dd, 1H, J=0.6 Hz, J=1.6 Hz). Mass spectrometry (APCI+) [M+H]⁺=324.

Compound of Formula (II-38)

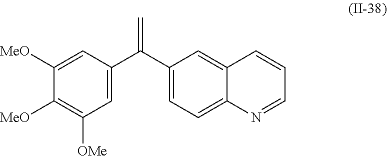

This compound was prepared following the operating mode described for formula (II-33) from 6-bromoquinolein. (Yield 47%).

¹H NMR: δ, ppm, CDCl₃, 300 MHz: 3.80 (s, 6H), 3.90 (s, 3H), 5.56 (s, 1H), 5.59 (s, 1H), 6.57 (s, 2H), 7.41 (dd, 1H, J=4.3 Hz, J=8.3 Hz), 7.75 (dd, 1H, J=2.0 Hz, J=8.7 Hz), 7.79 (d, 1H, J=1.6 Hz), 8.08 (d, 1H, J=8.7 Hz), 8.14 (dd, 1H, J=0.8 Hz, J=8.3 Hz), 8.91 (dd, 1H, J=1.6 Hz, J=4.2 Hz). Mass spectrometry (APCI+) [M+H]⁺=322.

Compound of Formula (II-39)

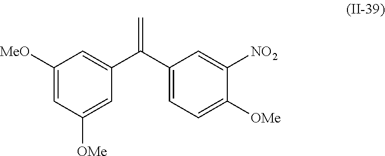

### Step 1: Synthesis of the Following Compound of Formula (XI-2):

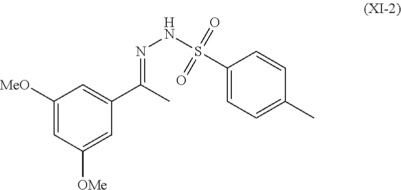

The formula (XI-2) compound was obtained following the preparation protocol described for compound (XI-1) from 3,5-dimethoxyacetophenone. (Yield 760).

¹H NMR: δ, ppm, CDCl₃, 300 MHz: 2.11 (s, 3H), 2.41 (s, 3H), 3.79 (s, 6H), 6.45 (t, 1H, J=2.1 Hz), 6.78 (dd, 2H, J=1.1 Hz), J=2.2 Hz), 7.30 (d, 2H, J=8.0 Hz), 7.92 (d, 2H, J=8.2 Hz). Mass spectrometry (ESI) [M+H]⁺=349.

### Step 2: Synthesis of Compound (II-39)

To a solution of XI-2 (1.2 mmol; 1.2 eq.), 196 mg f t-BuOLi (2.4 mmol; 2.4 eq.), 52 mg Pd₂ dba₃ (0.005 mmol; 10 mold), 98 mg X-Phos in 20 ml of dioxane is added a solution of 5-bromo-2-methoxy-nitrobenzene (1 mmol; 1 eq.) in 5 ml dioxane at room temperature. The reaction medium is then heated to 70° C. and the reaction is followed by TLC. After 6 h, the reaction medium is cooled to room temperature and diluted with CH₂Cl₂, filtered through celite and concentrated under reduced pressure. The crude product is purified on silica gel column (Cyclohexane/ethyl acetate—7:3). (Yield 80%).

¹H NMR: δ, ppm, CDCl₃, 300 MHz: 3.77 (s, 6H), 3.98 (s, 3H), 5.46 (d, J=5.33 Hz, 2H), 6.44 (m, 3H), 7.04 (d, J=8.73 Hz, 1H), 7.50 (dd, J=2.31 and 8.72 Hz, 1H), 7.86 (d, J=2.31 Hz, 1H).

Compound of Formula (II-40)

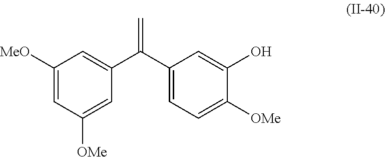

To a solution of XI-2 (1.2 mmol; 1.2 eq.), 196 mg of t-BuOLi (2.4 mmol; 2.4 eq.), 52 mg of Pd₂ dba₃ (0.005 mmol; 10 mol %), 98 mg of X-Phos in 20 ml of dioxane is added at room temperature a solution of 4-iodo-2-tert-butyldimethylsilyloxy anisole (1 mmol; 1 eq.) in 5 ml of dioxane. The reaction medium is then heated to 70° C. and the reaction is followed by TLC. After 6 h, the reaction medium is cooled to room temperature and diluted with CH₂Cl₂, then filtered through celite and concentrated under reduced pressure. The crude product is then desilylated in the presence of tetrabutylammonium fluoride (TBAF) following the protocol described for product II-34. The compound II-40 is purified on a silica gel column (Cyclohexane/ethyl acetate—7:3). (Yield 78%).

¹H NMR: δ, ppm, CDCl₃, 300 MHz: 3.78 (s, 6H), 3.88 (s, 3H), 5.36 (d, J=2.1 Hz, 1H), 5.41 (d, J=1 Hz, 1H), 5.72 (s, 3H), 6.46 (t, J=2.1 Hz, 1H), 6.52 (d, J=2.1 Hz, 1H), 6.77-6.83 (m, 2H), 6.99 (d, J=2.0 Hz, 1H). Mass spectrometry (ESI) [M+Na]⁺=309, (2M+Na]=595.

### 1.2 Synthesis of the Formula (I) Compounds of the Invention with X═CH

### General Protocol for Catalytic Reduction of Diarylethylenes:

1 mmol of diarylethylene is dissolved in 5 ml of ethyl acetate in the presence of 10 mol % of Pd/C. The whole is left to react under a hydrogen atmosphere until total disappearance of the starting product (TLC). The catalyst is filtered, the solvent is evaporated under reduced pressure and the residue obtained is passed through a silica gel chromatography column.

Compound of Formula (I-1) (Also Called Dihydro iso CA-4 or DHiCA-4 or isoerianine).

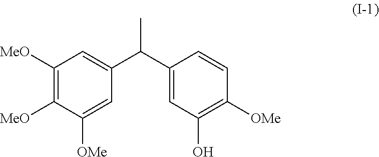

This was prepared following the general protocol from diarylethylene (II-2). Yield 98%.

¹H NMR: δ, ppm, CDCl₃, 300 MHz: 1.58 (d, 3H, J=7.2 Hz), 3.81 (s, 9H), 3.83 (s, 3H), 3.88 (q, 1H, J=7.2 Hz), 5.60 (s, 1H), 6.43 (s, 2H), 6.70 (dd, 1H, J=10.2 Hz, J=2.2 Hz), 6.78 (d, 1H, J=10.2 Hz), 6.81 (d, 1H, J=2.2 Hz). Mass spectrometry (ESI) [M+Na]⁺=341.

The two enantiomers (I-1a) and (I-1b) were separated on chiral column HPLC (column AD-H, P=621 psi, flow rate=1 ml/min; hexane/ethanol eluent: 75:25 (I-1a=8.5 min and I-1b=12.5 min).

Compound of Formula (I-2)

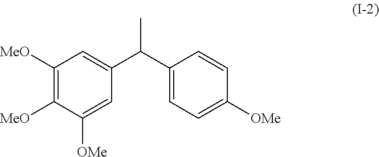

This was prepared following the general protocol from diarylethylene (II-3). (Yield 930).

¹H NMR: δ, ppm, CDCl₃, 300 MHz: 1.50 (d, 3H, J=7.2 Hz), 3.66 (s, 6H), 3.72 (s, 3H), 3.74 (s, 3H), 3.95 (q, 1H, J=7.2 Hz), 6.30 (s, 2H), 6.75 (d, 1H, J=7.2 Hz), 7.05 (d, 1H, J=10.2 Hz). Mass spectrometry (ESI) [M+Na]⁺=325.

Compound of Formula (I-3)

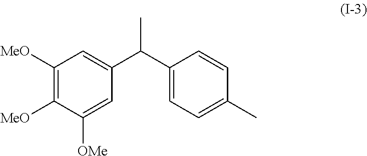

This was prepared following the general protocol from diarylethylene (II-4). Yield 990.

¹H NMR: δ, ppm, CDCl₃, 300 MHz: 1.52 (d, 3H, J=7.2 Hz), 2.20 (s, 3H), 3.70 (s, 6H), 3.72 (s, 3H), 3.94 (q, 1H, J=7.2 Hz), 6.35 (s, 2H), 6.98-7.05 (m, 4H). Mass spectrometry (ESI) [M+Na]⁺=309.

Compound of Formula (I-4)

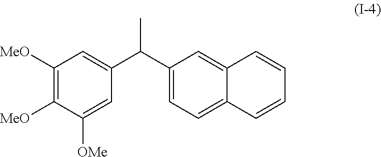

This was prepared following the general protocol from diarylethylene (II-5). Yield 91%.

¹H NMR: δ, ppm, CDCl₃, 300 MHz: 1.62 (d, 3H, J=7.2 Hz), 3.70 (s, 6H), 3.74 (s, 3H), 4.16 (q, 1H, J=7.2 Hz), 6.38 (s, 2H), 7.22 (dd, 1H, J=8.5 Hz, J=2.2 Hz), 7.28-7.42 (m, 2H), 7.61 (s, 1H), 7.64-7.77 (m, 3H). Mass spectrometry (EST) [M+Na]⁺=345.

Compound of Formula (I-5)

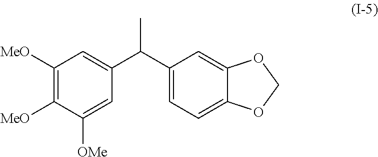

This was prepared following the general protocol from diarylethylene (II-6). Yield 86%.

¹H NMR: δ, ppm, CDCl₃, 300 MHz: 1.52 (d, 3H, J=7.2 Hz), 3.75 (s, 9H), 4.00 (q, 1H, J=7.2 Hz), 5.80 (s, 2H), 6.35 (s, 2H), 6.62-6.68 (m, 3H). Mass spectrometry (ESI) [M+Na]⁺=339.

Compound of Formula (I-6)

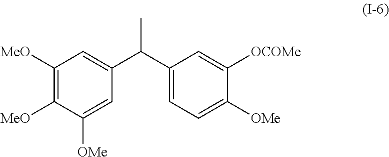

This was prepared following the general protocol from diarylethylene (II-7). Yield 90%.

¹H NMR: δ, ppm, CDCl₃, 300 MHz: 1.51 (d, 3H, J=7.2 Hz), 2.22 (s, 3H), 3.73 (s, 3H), 3.74 (s, 6H), 3.75 (s, 3H), 3.95 (q, 1H, J=7.2 Hz), 6.32 (s, 2H), 6.79-6.83 (m, 2H), 6.97 (dd, 1H, J=8.4 Hz, J=1.7 Hz). Mass spectrometry (ESI) [M+Na]⁺=383.

Compound of Formula (I-7)

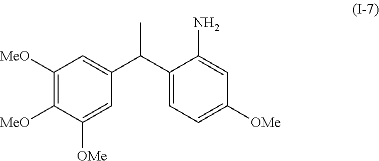

This was prepared following the general protocol from diarylethylene (II-10). Yield 86%.

¹H NMR: δ, ppm, CDCl₃, 300 MHz: 1.50 (d, 3H, J=7.2 Hz), 3.63 (s, 3H), 3.70 (s, 6H), 3.74 (s, 3H), 3.85 (q, 1H, J=7.2 Hz), 6.14 (m, 1H), 6.32 (m, 3H), 7.06 (d, 1H, J=8.4 Hz). Mass spectrometry (ESI) [M+H]⁺=318.

Compound of Formula (I-8)

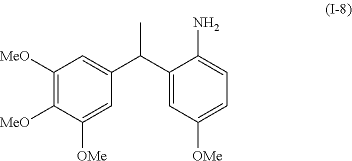

This was prepared following the general protocol from diarylethylene (II-11). Yield 89%.

¹H NMR: δ, ppm, CDCl₃, 300 MHz: 1.50 (d, 3H, J=7.2 Hz), 3.71 (s, 6H), 3.72 (s, 3H), 3.74 (s, 3H), 3.85 (q, 1H, J=7.2 Hz), 6.33 (s, 2H), 6.55 (d, 1H, J=8.4 Hz), 6.60 (dd, 1H, J=8.4 Hz, J=2.7 Hz), 6.80 (d, 1H, J=2.7 Hz). Mass spectrometry (ESI) [M+Na]⁺=340.

Compound of Formula (I-9)

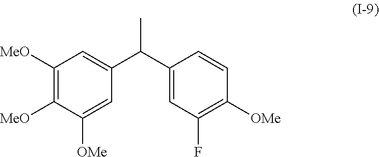

This was prepared following the general protocol from diarylethylene (II-12). Yield 97%.

¹H NMR: δ, ppm, CDCl₃, 300 MHz: 1.50 (d, 3H, J=7.2 Hz), 3.73 (s, 6H), 3.74 (s, 3H), 3.78 (s, 3H), 3.95 (q, 1H, J=7.2 Hz), 6.32 (s, 2H), 6.68-6.90 (m, 3H). Mass spectrometry (ESI) [M+Na]⁺=343.

Compound of Formula (I-10)

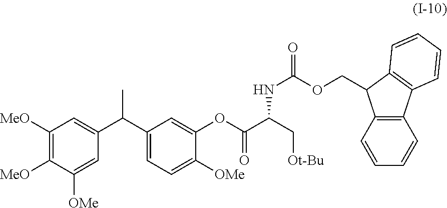

This was prepared following the general protocol from diarylethylene (II-13). Yield 91%.

¹H NMR: δ, ppm, CDCl₃, 300 MHz: 1.15 (s, 9H), 1.53 (d, 3H, J=7.2 Hz), 3.60-3.71 (m, 1H), 3.73 (s, 3H), 3.74 (s, 6H), 3.78 (s, 3H), 3.90-4.06 m, 2H), 4.21 (t, 1H, J=7.8 Hz), 4.31-4.49 (m, 2H), 4.72-4.79 (m, 1H), 5.70 (m, 1H), 6.30 (s, 2H), 6.60-6.83 (m, 2H), 6.95 (dd, 1H, J=8.4 Hz, J=2.7 Hz), 7.22 (t, 2H, J=7.4 Hz), 7.32 (t, 2H, J=7.4 Hz), 7.54 (m, 2H), 7.68 (d, 2H, J=7.4 Hz). Mass spectrometry (ESI) [M+Na]⁺=706.7.

Compound of Formula (I-11)

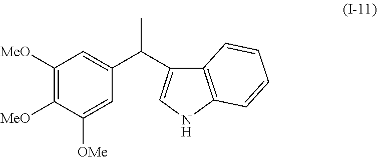

This was prepared following the general protocol from diarylethylene (II-16). Yield 79%.

¹H NMR: δ, ppm, CDCl₃, 300 MHz: 1.55 (d, 3H, J=7.2 Hz), 3.72 (s, 6H), 3.74 (s, 3H), 4.20 (q, 1H, J=7.2 Hz), 6.43 (s, 2H), 6.92-6.99 (m, 2H), 7.06-7.09 (dd, 1H, J=8.1 Hz, J=0.9 Hz), 7.15 (d, 1H, J=7.8 Hz), 7.27 (d, 1H, J=7.8 Hz), 7.95 (s, 1H). Mass spectrometry (ESI) [M+Na]⁺=334.

Compound of Formula (I-12)

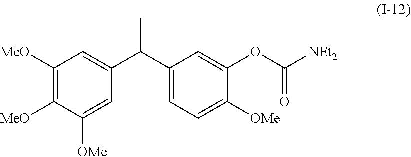

This was prepared following the general protocol from diarylethylene (II-14). Yield 91%.

¹H NMR: δ, ppm, CDCl₃, 300 MHz: 1.10-1.30 (m, 6H), 1.50 (d, 3H, J=7.2 Hz), 3.25-3.45 (m, 4H), 3.72 (s, 6H), 3.74 (s, 3H), 3.75 (s, 3H), 3.95 (q, 1H, J=7.2 Hz), 6.32 (s, 2H), 6.78 (d, 1H, J=8.4 Hz), 6.86-6.95 (m, 2H). Mass spectrometry (ESI) [M+Na]⁺=440.

Compound of Formula (I-13)

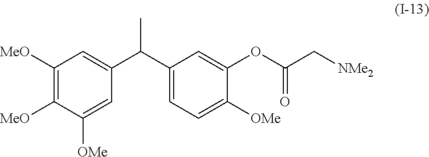

This was prepared following the general protocol from diarylethylene (II-15). Yield 93%.

¹H NMR: δ, ppm, CDCl₃, 300 MHz: 1.50 (d, 3H, J=7.2 Hz), 2.35 (s, 6H), 3.35 (s, 2H), 3.72 (s, 3H), 3.74 (s, 6H), 3.76 (s, 3H), 3.95 (q, 1H, J=7.2 Hz), 6.31 (s, 2H), 6.78-6.85 (m, 2H), 6.97 (dd, 1H, J=8.5 Hz, J=2.0 Hz). Mass spectrometry (ESI) [M+Na]⁺=426.

Compound of Formula (I-14)

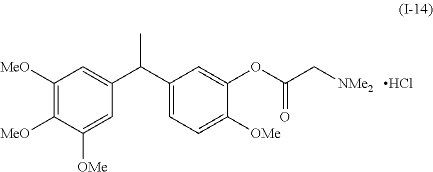

To a solution of 0.2 mmol of compound (I-13) in 1 ml of anhydrous methanol is added 1 ml of saturated HCl/MeOH solution. After stirring for 12 h at room temperature, the solvent is evaporated and the crude residue is taken up in ether. The solid formed is filtered through sintered glass then washed with ether. Yield 69%.

Elementary analyses: (MW=439.18). Calculated C, 60.06; H, 6.87; N, 3.18. Found C, 59.87; H, 6.74; N, 3.12.

Compound of Formula (I-15)

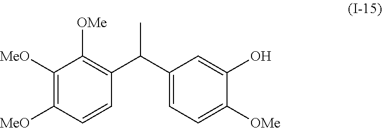

This was prepared following the general protocol from diarylethylene (II-17). Yield 84%.

¹H NMR: δ, ppm, CDCl₃, 300 MHz: 1.48 (d, 3H, J=7.2 Hz), 3.59 (s, 3H), 3.70 (s, 3H), 3.75 (s, 3H), 3.77 (s, 3H), 4.25 (q, 1H, J=7.2 Hz), 6.50-6.60 (m, 2H), 6.60-6.65 (d, 1H, J=8.3 Hz), 6.70 (d, 1H, J=1.9 Hz), 6.79 (d, 1H, J=8.6 Hz). Mass spectrometry (ESI negative) [M−H]⁻=317.

Compound of Formula (I-16)

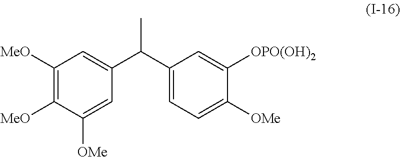

This was prepared following the general protocol from diarylethylene (II-8). Yield 70%.

¹H NMR: δ, ppm, CD₃OD, 300 MHz: 1.40 (m, 3H), 3.50 (s, 3H), 3.61 (s, 9H), 3.95 (m, 1H), 6.40 (m, 2H), 6.75-6.90 (m, 2H), 7.10-7.30 (m, 1H). Mass spectrometry (ESI negative) [M−H]⁻=397.

Compound of Formula (I-17)

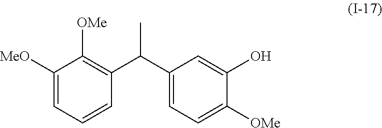

This was prepared following the general protocol from diarylethylene (II-18). Yield 81%.

¹H NMR: δ, ppm, CD₃OD, 300 MHz: 1.51 (d, 3H, J=7.1 Hz), 3.65 (s, 3H), 3.81 (s, 6H), 4.48 (q, 1H, J=7.2 Hz), 6.71-6.82 (m, 5H), 6.97 (t, 1H, J=8.0 Hz). Elementary analyses: (MW=288.14), Calculated C, 70.81; H, 6.99. Found C, 70.58; H, 6.94.

Compound of Formula (I-18)

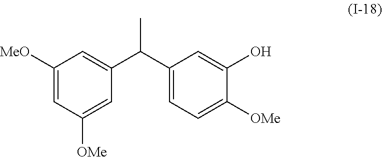

This was prepared following the general protocol from diarylethylene II-40. Yield 87%.

¹H NMR: δ, ppm, CDCl₃, 300 MHz: 1.52 (d, 3H, J=7.1 Hz), 3.65 (s, 3H), 3.81 (s, 6H), 4.48 (q, 1H, J=7.2 Hz), 6.71 (s, 1H), 6.71-6.81 (m, 5H), 6.98 (t, 1H, J=8.1 Hz). Elementary analyses: (MW=288.14) Calculated C, 70.81; H, 6.99. Found C, 70.74; H, 6.96.

Compound of Formula (I-19)

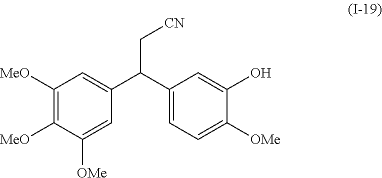

This was prepared following the general protocol from diarylethylene (II-19). Yield 50%.

¹H NMR: δ, ppm, CDCl₃, 300 MHz: 2.97 (d, 2H, J=7.5 Hz), 3.83 (s, 9H), 3.88 (s, 3H), 4.21 (t, 1H, J=7.6 Hz), 5.61 (s, 1H), 6.42 (s, 2H), 6.73-6.83 (m, 3H). Mass spectrometry (ESI) (M+Na]⁺=366.

Compound of Formula (I-20)

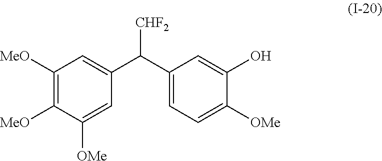

This was prepared following the general protocol from corresponding diarylethylene (II-20). Yield 62%.

¹H NMR: δ, ppm, CDCl₃, 300 MHz: 3.76 (s, 9H), 3.83 (s, 3H), 4.15 (td, 1H, J=15.8 Hz, J=4.2 Hz), 5.51 (s, 1H), 6.15 (td, 1H, J=55.9 Hz, J=4.2 Hz), 6.42 (s, 2H), 6.70-6.83 (m, 3H). Elementary analyses: (MW=354.35) Calculated C, 61.01; H, 5.68. Found C, 60.81; H, 5.46.

Compound of Formula (I-21)

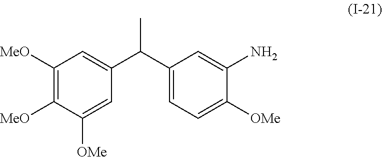

This was prepared following the general protocol from diarylethylene (II-9). Yield 58%.

¹H NMR: δ, ppm, CDCl₃, 300 MHz: 1.57 (d, 3H, J=7.2 Hz), 3.82 (s, 12H), 3.98 (q, 1H, J=7.2 Hz), 6.44 (s, 2H), 6.56 (d, 1H, J=2.1 Hz), 6.60 (dd, 1H, J=8.2 Hz, J=2.1 Hz), 6.72 (d, 1H, J=8.2 Hz). Mass spectrometry (ESI) [M+H]⁺=318.

Compound of Formula (I-22)

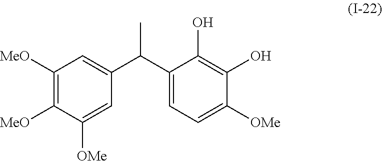

This was prepared following the general protocol from diarylethylene (II-34). Yield 95%.

¹H NMR: δ, ppm, CDCl₃, 300 MHz: 1.57 (d, 3H, J=7.2 Hz), 3.82 (s, 9H), 3.86 (s, 3H), 4.39 (q, 1H, J=7.0 Hz), 5.37 (s, 2H), 6.44 (d, 1H, J=8.6 Hz), 6.50 (s, 2H), 6.65 (d, 1H, J=8.6 Hz). Mass spectrometry SM (APCI, m/z,%): 335 (M+1, 100).

Compound of Formula (I-23)

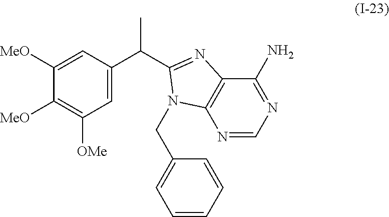

To a solution of II-21 in an AcOEt/MeOH mixture (4:1) is added 30 weight % PtO₂. The reaction medium is placed under vacuum using a filter pump then placed under an atmosphere of hydrogen. After 72 h at room temperature, the reaction medium is filtered through a sintered filter carrying a thin layer of celite eluting with AcOEt. After vacuum concentrating, the residue is purified by silica gel chromatography: cyclohexane/acetone (1:1). (Yield 30%).

¹H NMR: δ, ppm, CDCl₃, 300 MHz: 1.64 (d, 1H, J=7.0 Hz), 3.65 (s, 3H), 3.68 (s, 3H), 4.27 (q, 1H, J=7.0 Hz), 5.15 (d, J=16.2 Hz), 5.46 (d, J=16.2 Hz), 6.64 (bs, 2H), 7.03-7.23 (m, 1H), 8.18 (s, 1H). Mass spectrometry (positive ESI):[M+H]⁺=420.

Compound of Formula (I-24)

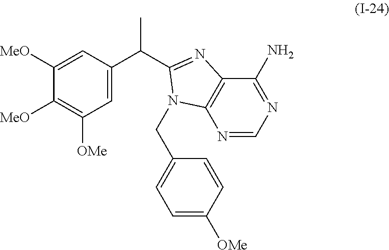

This compound is prepared following the same procedure as described for 1-23 from II-36. (Yield 39%).

¹H NMR: δ, ppm, CDCl₃, 300 MHz: 2.05 (s, 3H), 3.65 (s, 3H), 3.70 (s, 6H), 3.73 (s, 3H), 5.08 (d, 1H, J=15.8 Hz), 5.37 (d, 1H, J=15.8 Hz), 6.48 (s, 1H), 6.56 (s, 1H), 6.76-6.79 (m, 2H), 7.00-7.02 (m, 2H), 8.19 (s, 1H). Mass spectrometry (APCI⁺) [M+H]⁺=450.

Compound of Formula (I-25)

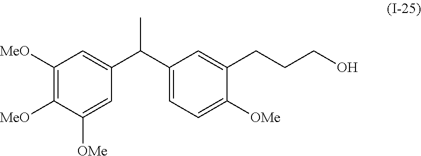

This was prepared following the general protocol from diarylethylene (II-23). (Yield 92%).

¹H NMR: δ, ppm, CDCl₃, 300 MHz: 1.59 (d, 3H, J=7.2 Hz), 1.83 (q, 2H, J=6.3 Hz), 2.69 (t, 2H, J=7.2 Hz), 3.59 (t, 2H, J=6.3 Hz), 3.81 (s, 9H), 3.82 (s, 3H), 4.00 (q, 1H, J=7.2 Hz), 6.42 (s, ²H), 6.8 (d, 1H, J=8.0 Hz), 7.01-7.04 (m, 2H). Mass spectrometry (APCI⁺) [M+H]⁺=361.

Compound of Formula (I-26)

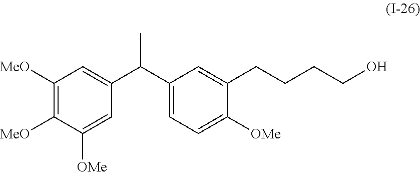

This was prepared following the general protocol from diarylethylene (II-24). (Yield 85%).

¹H NMR: δ, ppm, CDCl₃, 300 MHz: 1.58-1.63 (m, 7H), 2.61 (t, 2H, J=7.0 Hz), 3.66 (t, 2H, J=6.0 Hz), 3.79 (s, 3H), 3.81 (m, 9H), 3.70-4.06 (m, 1H), 6.42 (s, 2H), 6.76 (d, 1H, J=8.2 Hz), 6.96-7.02 (m, 2H). Mass spectrometry (APCI⁺) [M+H]⁺=375.

Compound of Formula (I-27)

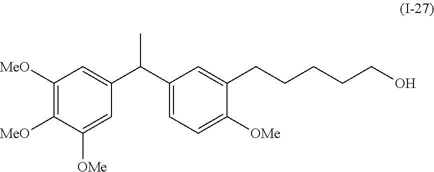

This was prepared following the general protocol from diarylethylene (II-25). (Yield 100%).

¹H NMR: δ, ppm, CDCl₃, 300 MHz: 1.36-1.43 (m, 2H), 1.60-1.66 (m, 7H), 2.56-2.61 (m, 2H), 3.62 (t, 2H, J=6.6 Hz), 3.80 (s, 3H), 3.81 (m, 9H), 3.97-4.05 (m, 1H), 6.43 (s, 2H), 6.76 (d, 1H, J=8.3 Hz), 6.97-7.03 (m, 2H). Mass spectrometry (APCI⁺) [M+H]⁺=389.

Compound of Formula (I-28)

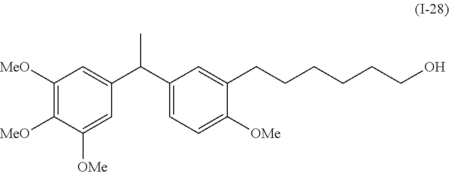

This was prepared following the general protocol from diarylethylene (II-26). (Yield 930).

¹H NMR: δ, ppm, CDCl₃, 300 MHz: 1.33-1.36 (m, 4H), 1.52-1.60 (m, 7H), 2.54-2.60 (m, 2H), 3.61 (t, 2H, J=6.6 Hz), 3.79 (s, 3H), 3.81 (s, 9H), 4.01 (q, 1H, J=7.3 Hz), 6.43 (s, 2H), 6.76 (d, 1H, J=8.3 Hz), 6.96 (d, 1H, J=2.5 Hz), 7.00 (dd, 1H, J=2.5 Hz, J=8.3 Hz). Mass spectrometry (APCI⁺) [M+H]⁺=403.

Compound of Formula (I-29)

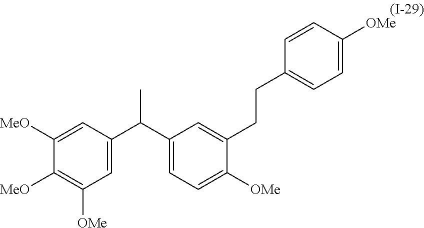

This was prepared following the general protocol from diarylethylene (II-27). (Yield 98%).

¹H NMR: δ, ppm, CDCl₃, 300 MHz: 1.56 (d, 3H, J=7.2 Hz), 2.76-2.89 (m, 4H), 3.79 (5, 3H), 3.80 (s, 3H), 3.81 (s, 6H), 3.82 (s, 3H), 3.99 (q, 1H, J=7.2 Hz), 6.42 (s, 2H), 6.77-6.82 (m, 3H), 6.94 (d, 1H, J=2.2 Hz), 7.02 (dd, 1H, J=2.2 Hz, J=8.4 Hz), 7.10 (d, 2H, J=8.6 Hz). Mass spectrometry (APCI⁺) [M+H]⁺=437.

Compound of Formula (I-30)

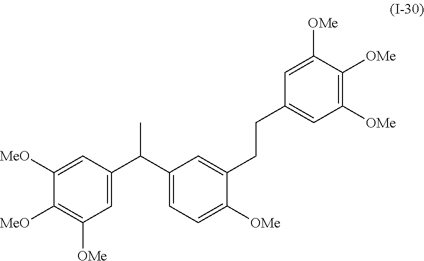

This was prepared following the general protocol from diarylethylene (II-28). (Yield 98%).

¹H NMR: δ, ppm, CDCl₃, 300 MHz: 1.56 (d, 3H, J=7.2 Hz), 2.76-2.91 (m, 4H), 3.81 (m, 21H), 3.95-4.05 (m, 1H), 6.38 (s, 2H), 6.42 (s, 2H), 6.79 (d, 1H, J=8.4 Hz), 6.93 (d, 1H, J=2.1 Hz), 7.01-7.05 (m, 1H₅). Mass spectrometry (APCI⁺) [M+H]⁺=497.

Compound of Formula (I-31)

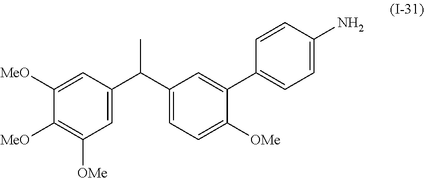

This was prepared following the general protocol from diarylethylene (II-29). (Yield 88%).

¹H NMR: δ, ppm, CDCl₃, 300 MHz: 1.62 (d, 3H, J=7.2 Hz), 3.78 (s, 3H), 3.82 (s, 9H), 4.02-4.10 (m, 3H), 6.46 (s, 2H), 6.74 (d, 2H, J=8.5 Hz), 6.87 (d, 1H, J=8.4 Hz), 7.10 (dd, 1H, J=2.2 Hz, J=8.4 Hz), 7.17 (d, 1H, J=2.2 Hz), 7.33 (d, 2H, J=8.5 Hz). Mass spectrometry (APCI⁺) [M+H]⁺=394.

Compound of Formula (I-32)

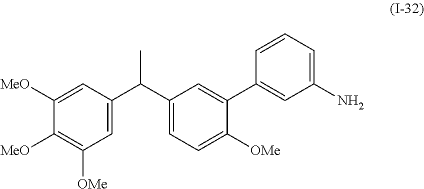

This was prepared following the general protocol from diarylethylene (II-30). (Yield 89%).

¹H NMR: δ, ppm, CDCl₃, 300 MHz: 1.62 (d, 3H, J=7.2 Hz), 3.78 (s, 3H), 3.82 (s, 11H), 4.03-4.09 (m, 1H), 6.45 (s, 2H), 6.64-6.68 (m, 1H), 6.84 (m, 1H), 6.89 (d, 2H, J=8.4 Hz), 7.12-7.19 (m, 3H). Mass spectrometry (APCI⁺)[M+H]⁺=394.

Compound of Formula (I-33)

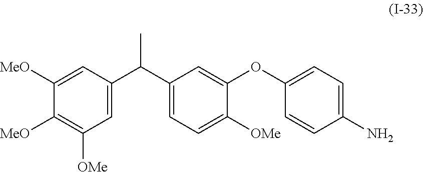

This was prepared following the general protocol from diarylethylene (II-31). (Yield 85%).

¹H NMR: δ, ppm, CDCl₃, 300 MHz: 1.52 (d, 3H, J=7.2 Hz), 3.79 (s, 6H), 3.81 (s, 5H), 3.84 (s, 3H), 3.90-3.98 (m, 1H), 6.35 (s, 2H), 6.62 (d, 2H, J=8.9 Hz), 6.76-6.81 (m, 3H), 6.88 (m, 2H, H₅, H₆). Mass spectrometry (APCI⁺) [M+H]⁺=410.

Compound of Formula (I-34)

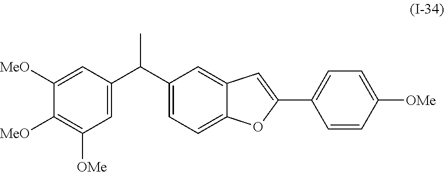

This was prepared following the general protocol from diarylethylene (II-32). (Yield 98%).

¹H NMR: δ, ppm, CDCl₃, 300 MHz: 1.68 (d, 3H, J=7.2 Hz), 3.82 (s, 6H), 3.83 (s, 3H), 3.86 (s, 3H), 4.19 (q, 1H, J=7.2 Hz), 6.48 (s, 2H), 6.84 (s, 1H), 6.97 (d, 2H, J=9.0 Hz), 7.10-7.14 (m, 1H), 7.40-7.42 (m, 2H), 7.77 (d, 2H, J=9.0 Hz). Mass spectrometry (APCI⁺) [M+H]⁺=419.

Compound of Formula (I-35)

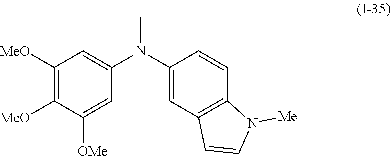

This was prepared following the general protocol from diarylethylene (II-37). (Yield 590).

¹H NMR: δ, ppm, CDCl₃, 300 MHz: 1.67 (d, 3H, J=7.2 Hz), 3.74 (s, 3H), 3.76 (s, 9H), 4.19 (q, 1H, J=7.1 Hz), 6.38 (dd, 1H, J=0.6 Hz, J=3.0 Hz), 6.55 (s, 2H), 7.05 (dd, 1H, J=1.4 Hz, J=8.5 Hz), 7.10 (d, 1H, J=3.1 Hz), 7.26 (d, 1H, J=8.5 Hz), 7.45 (s, 1H). Mass spectrometry (APCI⁺) [M+H]⁺=326.

Compound of Formula (I-36)

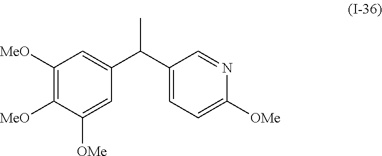

A solution of 30 mg of II-35 (0.099 mmol; 1 eq.) in 4.5 ml of methanol is hydrogenated by an H-Cube (continuous flow hydrogenator. Conditions: 1 ml/min, room temperature). The collected filtrate is concentrated under reduced pressure. The crude product is purified by silica gel chromatography (cyclohexane/diethylether—1:1, Rf=0.27). (Yield 99%).

¹H NMR: δ, ppm, CDCl₃, 300 MHz: 1.63 (d, 3H, J=7.2 Hz), 3.75 (s, 3H), 3.81 (s, 6H), 3.89 (s, 3H), 4.11 (q, 1H, J=7.2 Hz), 6.55 (s, 2H), 6.75 (d, 1H, J=8.6 Hz), 7.59 (dd, 1H, J=2.3 Hz, J=8.6 Hz), 8.04 (d, 1H, J=2.3 Hz). Mass spectrometry (APCI⁺) [M+H]⁺=304.

Compound of Formula (I-37)

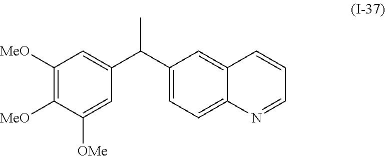

A solution of 26 mg of II-38 (0.081 mmol; 1 eq.) in 4 ml methanol is hydrogenated by the H-Cube (continuous flow hydrogenator. Conditions: 1 ml/min, room temperature). The collected filtrate is concentrated under reduced pressure. The crude product is purified on silica gel column (diethylether. Rf=0.24). (Yield 54%).

¹H NMR: δ, ppm, CDCl₃, 300 MHz: 1.76 (d, 3H, J=7.2 Hz), 3.76 (s, 3H), 3.80 (s, 6H), 4.36 (q, 1H, J=7.0 Hz), 6.61 (s, 2H), 7.53 (dd, 1H, J=4.4 Hz, J=8.3 Hz), 7.70 (dd, 1H, J=1.8 Hz, J=8.8 Hz), 7.87 (s, 1H), 7.96 (d, 1H, J=8.8 Hz), 8.36 (d, 1H, J=8.0 Hz), 8.81 (dd, 1H, J=1.5 Hz, J=4.3 Hz). Mass spectrometry (APCI⁺) [M+H]⁺=324.

Compound of Formula (I-38)

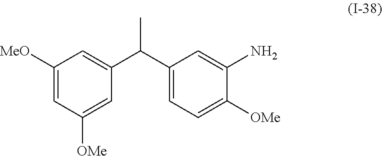

This was prepared following the general protocol from corresponding diarylethylene II-39. (Yield 65%).

¹H NMR: δ, ppm, CDCl₃, 300 MHz: 1.56 (d, 3H, J=7.2 Hz), 3.76 (s, 6H), 3.82 (s, 3H), 3.95 (q, 1H, J=7.2 Hz), 6.29 (t, 1H, J=2.3 Hz), 6.39 (d, 2H, J=2.3 Hz), 6.57 (d, 1H, J=2.1 Hz), 6.61 (dd, 1H, J=2.1 Hz, J=8.2 Hz), 6.71 (d, 1H, J=8.2 Hz). Mass spectrometry (APCI⁺) [M+H]⁺=288.

### 1.3 Synthesis of the Intermediate Compounds of Formula (X)

These compounds were prepared by coupling 3,4,5-trimethoxyaniline with the corresponding halide following the general procedure described in the following reference: Antimitotic and cell growth inhibitory properties of combretastatin A-4-like ethers. Lawrence, N. J.; Rennison, D.; Woo, M.; McGown, A. T.; Hadfield, J. A. Bioorg. Med. Chem. Lett. 2001, 11, 51-54.

General procedure: To a mixture of Pd(OAc)₂ (7.5 mg, 0.05 mmol, 5 mol %), Xantphos (29 mg, 0.05 mmol, 5 mol %), aryl or heteroaryl bromide (1.0 mmol), 3,4,5-trimethoxyaniline (1.5 mmol) and CS₂CO₃ (651 mg, 2 mmol), dioxane (2 ml) is added under a stream of argon. The tube is sealed and the mixture is heated to 100° C. overnight. The mixture is cooled, filtered through celite, then washed with ethyl acetate. The solvent is evaporated and the residue is passed through a silica gel chromatography column.

Compound of Formula (X-1)

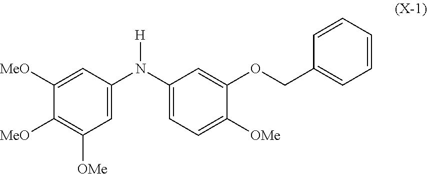

This was prepared following the general procedure for coupling using 2-benzyloxy-4-iodoanisole. (Yield 61%).

¹H NMR: δ, ppm, CDCl₃, 300 MHz: 3.12 (s, 3H), 3.66 (s, 6H), 3.75 (s, 3H), 3.80 (s, 3H), 5.02 (s, 2H), 6.06 (s, 2H), 6.57 (dd, 1H, J=2.4 Hz, J=8.4 Hz, 6.63 (d, 1H, J=2.4 Hz), 6.76 (d, 1H, J=8.4 Hz), 7.18-7.35 (m, 5H). Mass spectrometry (ESI)[M+Na]⁺=419.

Compound of Formula (X-2)

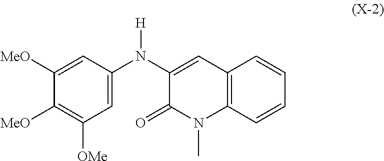

This was prepared following the general procedure for coupling using 3-bromo-N-methyl-2-quinolone. (Yield 88%).

¹H NMR: δ, ppm, CDCl₃, 300 MHz: 3.76 (s, 6H), 3.78 (s, 3H), 6.47 (se, 1H), 6.53 (s, 2H), 6.65 (t, 1H, J=8.2 Hz), 6.75 (d, 1H, J=8.4 Hz), 7.41 (t, 1H, J=8.2 Hz), 8.12 (d, 1H, J=4.8 Hz). Mass spectrometry (ESI) [M+H]⁺=341.

Compound of Formula (X-3)

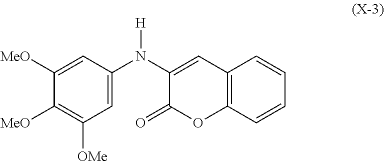

This was prepared following the general procedure for coupling using 3-bromo-coumarin. (Yield 83%).

¹H NMR: δ, ppm, CDCl₃, 300 MHz: 3.79 (s, 9H), 6.40 (s, 2H), 6.59 (s, 1H), 7.00 (s, 1H), 7.13-7.25 (m, 4H). Mass spectrometry (ESI)[M+H]⁺=328.

Compound of Formula (X-4)

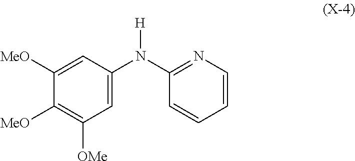

This was prepared following the general procedure for coupling using the corresponding 3-bromo-pyridine. (Yield 69%).

¹H NMR: δ, ppm, CDCl₃, 300 MHz: 3.76 (s, 6H), 3.78 (s, 3H), 6.47 (se, 1H), 6.63-6.67 (m, 1H), 6.75 (d, 1H, J=8.4 Hz), 7.39-7.44 (m, 1H), 8.12 (d, 1H, J=4.8 Hz). Mass spectrometry (APCI) [M+H]⁺=261.

Compound of Formula (X-5)

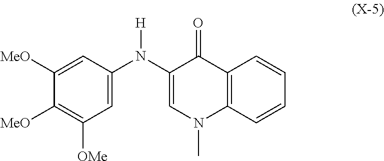

This was prepared following the general procedure for coupling using the corresponding 3-bromo-N-methyl-4-quinolone. (Yield 80%).

¹H NMR: δ, ppm, CDCl₃, 300 MHz: 3.82 (s, 3H, 3.83 (s, 6H), 3.84 (s, 3H), 6.37 (s, 2H), 7.35 (t, 2H, J=7.5 Hz), 7.41 (dd, 2H, J=2.7 Hz, J=8.4 Hz), 7.70 (s, 2H), 8.52 (d, 1H, J=8.4 Hz). Mass spectrometry (ESI) [M+H]⁺=341.

### 1.4 Synthesis of the Formula (I) Compounds of the Invention with X═N

### General Procedure for Alkylation or Acylation of the Formula (X) Compounds.

To a solution of 0.14 mmol of diarylaniline or aryl-heteroarylaniline in 2 ml of DMF is added 0.28 mmol of sodium hydride (2 eq.). After stirring for 20 min at room temperature, 0.28 mmol of methyl iodide or acetyl chloride are added. The whole is stirred 3 hours at room temperature before being hydrolysed with a 1M solution of hydrochloric acid (3 ml). The organic phase is separated and the aqueous phase is extracted with ethyl acetate (2×10 ml). The organic phases are combined, dried over sodium sulphate, and concentrated to give a residue which is purified on silica gel.

Compound of Formula (I-39)

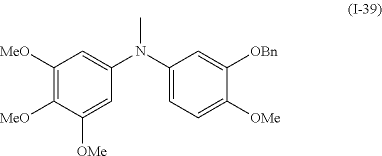

This was prepared by alkylation of secondary diarylamine (prepared according to Lawrence N. J.; Rennison, D.; Woo, M.; McGown, A. T.; Hadfield, J. A. Bioorg. Med. Chem. Lett. 2001, 11, 51-54) following the general procedure. Yield 61%.

¹H NMR: δ, ppm, CDCl₃, 300 MHz: 3.12 (s, 3H), 3.65 (s, 6H), 3.73 (s, 3H), 3.81 (s, 3H), 5.00 (s, 2H), 5.97 (s, 2H), 6.55-6.59 (m, 2H), 6.78 (d, 1H, J=8.4 Hz), 7.18-7.30 (m, 5H). Mass spectrometry (ESI) [M+Na]⁺=432.

Compound of Formula (I-40)

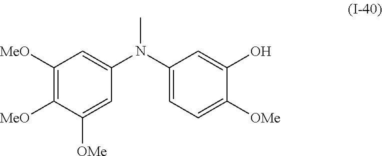

1 mmol of 1-39 is dissolved in 5 ml of ethyl acetate in the presence of 10 mol % of Pd/C. The whole is left to react under a hydrogen atmosphere until total consumption of the starting product (TLC). The catalyst is filtered then the solvent is evaporated under reduced pressure and the residue obtained is passed through a silica gel chromatography column. Yield 98%.

¹H NMR: δ, ppm, CDCl₃, 300 MHz: 3.22 (s, 3H), 3.76 (s, 6H), 3.80 (s, 3H), 3.86 (s, 3H), 5.40-5.80 (se, 1H), 6.14 (s, 2H), 6.53 (dd, 1H, J=8.7 Hz), J=2.7 Hz), 6.66 (d, 1H, J=2.7 Hz), 6.79 (d, 1H, J=8.7 Hz). Mass spectrometry (ESI) [M+Na]⁺=342.

Compound of Formula (I-41)

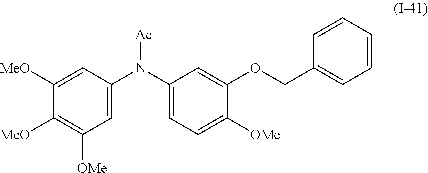

This was prepared following the general procedure for acylation from X-1. Yield 54%.

¹H NMR: δ, ppm, CDCl₃, 300 MHz: 1.96 (s, 3H), 3.67 (s, 6H), 3.74 (s, 3H), 3.80 (s, 3H), 5.06 (s, 2H), 6.33 (s, 2H), 6.69-6.80 (m, 3H), 7.18-7.27 (m, 5H). Mass spectrometry (ESI) [M+H]⁺=438.

Compound of Formula (I-42)

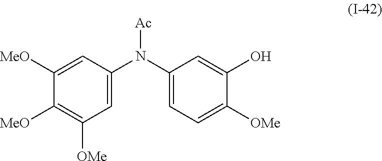

1 mmol of 1-41 is dissolved in 5 ml of ethyl acetate in the presence of 10 mol % of Pd/C. The whole is left to react under a hydrogen atmosphere until complete consumption of the starting product (TLC). The catalyst is filtered, then the solvent is evaporated under reduced pressure and the residue obtained is passed through a silica gel chromatography column. Yield 93%.

¹H NMR: δ, ppm, CDCl₃, 300 MHz: 2.00 (s, 3H), 3.73 (s, 9H), 3.81 (s, 3H), 5.90 (se, 1H), 6.42 (s, 2H), 6.74-6.79 (m, 3H). Mass spectrometry (ESI) [M+H]⁺=348.

Compound of Formula (I-43)

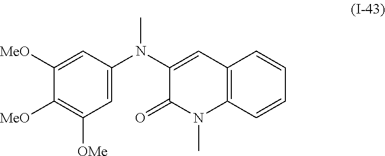

This was prepared following the general procedure for alkylation from X-2. Yield 80%.

¹H NMR: δ, ppm, CDCl₃, 300 MHz: 3.24 (s, 3H), 3.69 (s, 9H), 3.73 (s, 3H), 6.11 (s, 2H), 7.14-7.19 (m, 1H), 7.27-7.30 (m, 1H), 7.34 (s, 1H), 7.40-7.44 (m, 2H). Mass spectrometry (ESI) [M+H]⁺=355.

Compound of Formula (I-44)

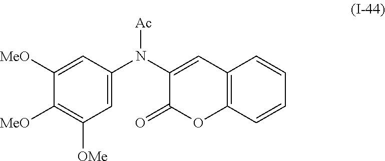

This was prepared following the general procedure for acylation from X-3. Yield 43%.

¹H NMR: δ, ppm, CDCl₃, 300 MHz: 2.06 (s, 3H), 3.78 (s, 3H), 3.79 (s, 6H), 6.63 (s, 2H), 7.20-7.30 (m, 2H), 7.38-7.46 (m, 2H), 7.60 (s, 1H). Mass spectrometry (ESI) [M+H]⁺=370.

Compound of Formula (I-45)

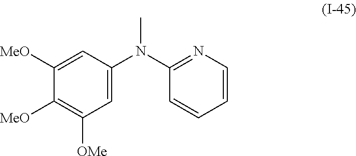

This was prepared following the general procedure for alkylation from X-4. Yield 71%.

¹H NMR: δ, ppm, CDCl₃, 300 MHz: 3.37 (s, 3H), 3.75 (s, 3H), 3.80 (s, 6H), 6.41-6.44 (m, 3H), 6.53 (td, 1H, J=5.7 Hz, J=0.6 Hz), 7.22-7.28 (m, 1H), 8.14-8.16 (m, 1H). Mass spectrometry (ESI)=275

Compound of Formula (I-46)

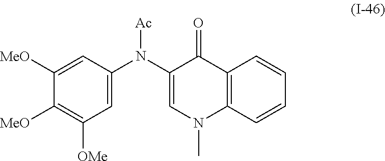

This was prepared following the general procedure for acylation from X-5. Yield 54%.

¹H NMR: δ, ppm, CDCl₃, 300 MHz: 2.07 (s, 9H), 2.11 (s, 3H), 3.75 (s, 6H), 3.78 (s, 3H), 6.64 (s, 1H), 6.75 (s, 1H), 7.34-7.37 (m, 2H), 7.64-7.71 (m, 2H), 8.47 (m, 1H). Mass spectrometry (ESI) [M+H]⁺=383.

### Example 2

### In Vitro Study of the Cytotoxicity of the Compounds of the Invention

The effects on the proliferation of different cancer cells and the proliferation of endothelial cells were studied.

The biological activity of the compounds of the invention was studied in vitro on 7 human cancer cell lines of different tissular origin (HCT116: coloreactal carcinoma; K562: chronic myeloid leukaemia; B16-F10: melanoma; U87: glioblastoma; H1299: non-small cell lung cancer and MDA-MB 231 and MDA-MB 435: breast cancer). The cells chosen for this study were incubated at 37° C. in the presence of one of the compounds added to the culture medium at different concentrations. All the experiments conducted allowed determination of the extent of toxicity of the tested compound, effect thereof on the cell cycle process and capacity thereof to induce cell death by apoptosis.

The cancer cell lines were obtained from the American Type Culture Collection (Rockville, Md., USA) and were cultured following the supplier's recommendations.

The cells H1299, U87, MDA-MB231, MDA-MB435 and B16F10 were cultured in Dulbecco Minimal Essential Medium (DMEM) containing 4.5 g/l glucose, supplemented with 10% foetal calf serum and 1% glutamine. The K562 and HCT116 cells were cultured in RPMI 1640 medium containing 10% foetal calf serum and 1% glutamine. All the cell lines were held in culture at 37° C. in a humid atmosphere containing 5% CO₂. Cell viability was evaluated using the reagent CellTiter-Blue™ (Promega, Wis., USA) paying heed to the manufacturer's instructions. The cells were seeded in 96-well culture plates to the proportion of 5000 cells per well in 50 μl culture medium. After 24 hours of culture, the compounds of general formula (I) dissolved in DMSO were added individually to each of the wells to the proportion of 50 μl per well. All the compounds were tested in triplicate for each defined concentration, and each experiment was repeated 3 times. After 72 hours of incubation, 20 μl resazurin were added to each well. After 2 hours of incubation, the emitted fluorescence was measured at 590 nm after excitation at 560 nm using a fluorescence reader of Victor type (Perkin-Elmer, USA).

The concentration of each of the compounds which induces the death of 50% of cells (IC₅₀) was determined after 72 hours of incubation. Some compounds conforming to the invention show an IC₅₀ of nanomolar order. The results obtained are given in following Table 1.

It is notably ascertained that the two enantiomers (I-1a) and (I-1b) have the same cytotoxic activity.

TABLE 1

| Molecules of | IC₅₀ for different cell lines (nM) | | |
|---|---|---|---|
| the invention | HCT116 | H1299 | M231 |
| (I-1) | 50-65 | — | 40 |
| (I-1a) | 60 | 35 | — |
| (I-1b) | 50 | 30 | — |
| (I-9) | 85 | — | — |
| (I-16) | 90^{a} | — | 50^{a} |
| | 100^{b} | | 50^{b} |
| (I-14) | 50^{a} | — | 50^{a} |
| | 90^{b} | | 50^{b} |
| (I-19) | 80 | — | — |
| (I-20) | 30 | — | — |
| (I-21) | 60 | — | — |

— means that no measurement was made.
^{a}in DMSO;
^{b}in water

### Example 3

### Study of the Inhibition of Tubulin Polymerization

Tests on the inhibition of tubulin polymerization were conducted on the compounds which exhibited the best cytotoxic activity. These tests were conducted on tubulin purified using the Shelanski method (Shelanski, M. C.; Gaskin, F.; Cantor, C. R. Proc. Natl. Acad. Sci. USA, 1973, 70, 765-768) from porcine brain in which it forms 20 to 25% of soluble proteins. The purification method is based on temperature-dependent assembly-disassembly cycles. Polymerisation of tubulin was followed by turbidimetry using the Gaskin method (Gaskin, F.; Cantor, C. R.; Shelanski, M. L.; J. Bio. Mol., 1974, 89, 737) at a wavelength of 350 nm. The different samples were dissolved in DMSO and incubated 10 minutes at 37° C. then 5 minutes at 0° C.

The compound CA-4 and DMSO were used as reference.

The tests, for these compounds, showed inhibiting activity of tubulin polymerisation similar to that of the reference compound CA-4 (of the order of one micromolar to a few tens of micromolars only). The results obtained are given in following Table 3.

It is also ascertained that the two enantiomers (I-1a) and (I-1b) have the same capacity to inhibit tubulin polymerisation.

TABLE 3

| Molecules of the | Tubulin inhibition |
|---|---|
| invention | (IC₅₀ in μM) |
| (I-1) | 3.2 |
| (I-1a) | 5.0-6.0 |
| (I-1b) | 6.7-8.0 |
| (I-9) | 6.0 |
| (I-18) | 11 |
| (I-38) | 11 |

### Example 4

### Study of Anti-Vascular Activity

### 4.1 In Vitro Study of Cytotoxicity on Human Endothelial Cells

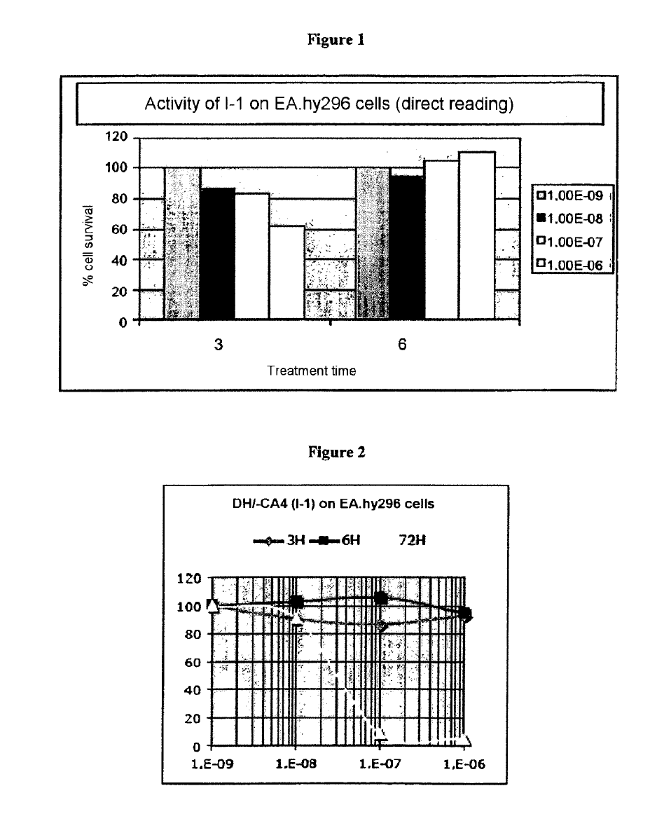

The cytotoxicity of compound (I-1) against human endothelial cells (EAhy926) was evaluated after 3, 6 or 72 hours of treatment. The number of living cells was counted either immediately after treatment lasting 3 or 6 hours (FIG. 1), or 72 hours after halting treatment lasting 3, 6 or 72 hours (FIG. 2). It is observed that when the endothelial cells are treated for 72 hours with compound (I-1), the IC₅₀ is 50 nM. On the other hand, after 3 hours of treatment, compound (I-1) shows no cytotoxic activity even at the dose of 10 nM.

### 4.2 In Vitro Study on the Formation of Vascular Tubes on Matrigel®

To determine whether compound (I-1) or (I-16) perturbs the spatial organisation of endothelial cells into structures similar to vascular capillaries, human endothelial cells (EAhy926) were treated immediately after being placed in culture on Matrigel®, or after a culture time of 24 hours to allow them to form vascular tubes.

The EAhy926 cells (immortalized HUVEC macro-vascular endothelial cells) were cultured in Dulbecco Minimal Essential Medium (DMEM) containing 4.5 g/l glucose supplemented with 10% foetal calf serum, 1% glutamine and HAT supplement (100 μM of hypoxanthine, 0.4 μM of aminopterine and 16 μM of thymidine, Invitrogen; Cergy-Pontoise, France). The cells were held in culture at 37° C. in a humid atmosphere containing 5% CO₂.

The cells were seeded in 96-well culture plates to the proportion of 3000 cells per well in 50 μl culture medium. After 24 hours of incubation, compound (I-1) was added at different concentrations for 1 hour, 3 hours, 6 hours or 72 hours. At the end of the treatment, the number of cells was evaluated using the reagent CellTiter-Blue™ (Promega, Wis., USA) as described previously. In parallel, after 1 hour, 3 hours or 6 hours of treatment with compound (I-1) the culture medium was removed and replaced by fresh medium for 72 hours and the number of living cells was then measured using the CellTiter-Blue™ reagent.

To evaluate the anti-vascular activity of compounds (I-1) and (I-16), the EAhy926 cells were placed in culture in 96-well culture plates previously coated with an extract of extracellular matrix (Matrigel™, BD Biosciences, Le Pont-de-Claix, France) in which they spontaneously form capillary tubes.

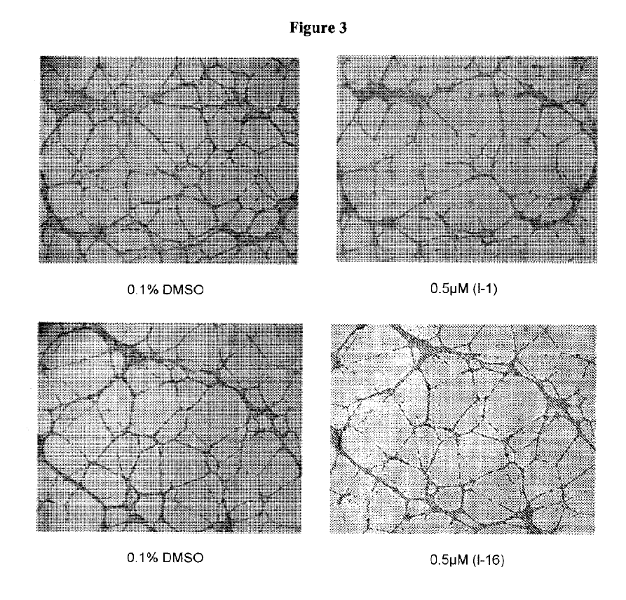

Firstly, we measured the capacity of compounds (I-1) and (I-16) to inhibit the formation of the capillary network. The Matrigel™ is deposited in 96-well culture plates to the proportion of 70 μl/well and left to incubate at 37° C. for 45 minutes to allow polymerization thereof. 15,000 cells in suspension in 150 μl of culture medium were seeded per well in each of the wells containing Matrigel™, in the absence or presence of different concentrations of compound (I-1) or (I-16) (0.5 μM or 1 μM), to the proportion of 3 wells per concentration. After 3 hours of incubation at 37° C., the cells were observed and photographed using an optical microscope of TE2000 type (Nikon, France) equipped with a camera (FIG. 3).

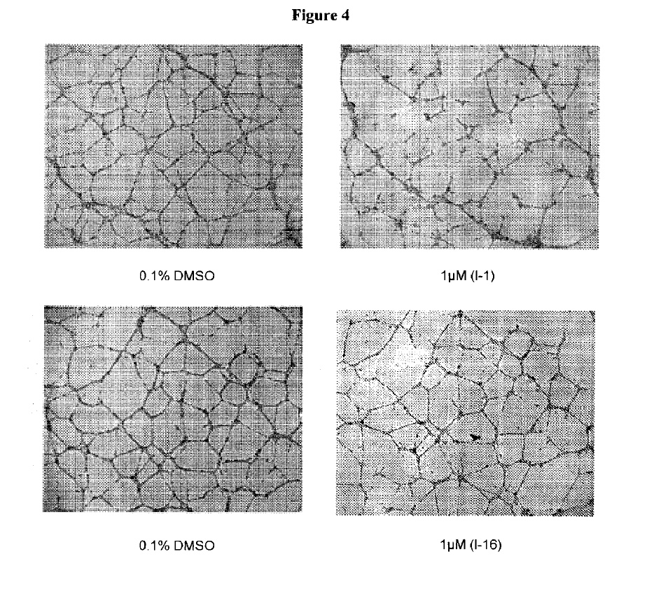

In parallel, 15,000 EAhy926 cells in suspension in 150 μl of culture medium were seeded in each of the wells containing Matrigel™. After 24 hours of incubation, when the capillary network is well formed, compound (I-1) or (I-16) was added at different concentrations (0.5 μM or 1 μM). The effect of the product was observed and photographed after 3 hours of incubation using an optical microscope (FIG. 4).

After a treatment time of 3 hours at a dose of 0.5 μM or 1 μM (non-toxic) it is observed that compounds (I-1) or (I-16) induce a major decrease in the number of vascular tubes. These results indicate that compounds (I-1) and (I-16) also have an anti-vascular activity that is potentially useful in therapeutics.

1. Compound of following formula (I): 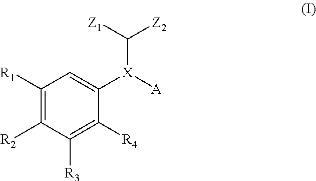
wherein:
R₁ and R₂ each independently represent a methoxy group optionally substituted with one or more fluorine atoms,
R₂ and R₄ each independently represent a hydrogen atom or a methoxy group optionally substituted with one or more fluorine atoms,
A is a cycle chosen from the group comprising aryl and heteroaryl groups, said heteroaryls being chosen from among quinolyl, isoquinolyl, imidazolyl, indolyl, benzothiophenyl, benzofuranyl, benzoimidazolyl, purinyl, pyridinyl, pyridazinyl, pyrimidinyl, pyrazinyl, pyrrolyl, furanyl and thiophenyl groups, said cycle possibly being:
either adjoined to a 6-membered heterocycle, optionally comprising one or more unsaturations and optionally substituted with one or more C₁ to C₄ alkyl groups and/or with an oxo group,
or substituted with one or more groups chosen from among halogen atoms, the groups —B(OH)₂, C₁ to C₆ alkyl optionally substituted with OH, C₂ to C₄ alkenyl, C₂ to C₄ alkynyl, aryl, heteroaryl, aryloxy, aryl-(C₁ to C₄ alkyl), —COOH, —NO₂, —NR₇R₈, —NHCOR₇, —CONR₇R₈, —NHCOOR₉, —OSi(C₁-C₄ alkyl)₃, —NHSO₂R₉, C₁ to C₄ alcoxy optionally substituted with one or more fluorine atoms, —OCONR₇R₈, —OSO₂CF₃, —OSO₂R₉, —SO₂R₉, —SO₃R₉, —OSO₃H, —OPO(OR₁₀)₂, —ONR₇R₈, —OR₁₁, —SO₂NR₁₂R₁₃, —SO₂NHCOR₁₄, —OCOR₁₅, —OCOOR₁₆, —SR₁₇ and a residue of a molecule with anti-tumour activity bound via an ester or amide bond, the aryl rings of said groups optionally being substituted with one or more OH, C₁ to C₄ alkoxy, NR₇R₈ groups,
X represents a nitrogen atom or a CH group and advantageously represents a CH group,
Z₁ represents a hydrogen atom or a fluorine atom, and
Z₂ represents a hydrogen atom, a fluorine atom, a C₁ to C₄ alkyl, —CN, —SO₃R₉, —COOR₁₅ or —COR₁₅ group,
wherein
R₇ and R₈ each independently represent a hydrogen atom or a C₁ to C₄ alkyl, aryl or heteroaryl group, and advantageously represent a hydrogen atom or C₁ to C₄ alkyl group,
R₉ represents a C₁ to C₄ alkyl, aryl or heteroaryl group, and advantageously represents a C₁ to C₄ alkyl group,
R₁₀ represents a hydrogen atom or a C₁ to C₄ alkyl group or a benzyl group,
R₁₁ represents a hydrogen atom, an O-protecting group, a sugar, an amino-sugar, or amino acid, the free OH and NH₂ groups of sugars, amino-sugars and amino acids possibly being substituted with an O-protecting and N-protecting group respectively,
R₁₂ and R₁₃ each independently represent a hydrogen atom or a C₁ to C₄ alkyl, aryl or heteroaryl group,
R₁₄ represents a —CO—(C₁ to C₄ alkyl) group or the residue of an amino acid molecule attached to the —SO₂NH— group via its carboxylic acid function,
R₁₅ represents a hydrogen atom, a C₁ to C₄ alkyl, aryl or heteroaryl group, or a —(CH₂)ₘCO₂H or —(CH₂)ₘNR₇R₈ group where m represents an integer of between 1 and 3,
R₁₆ represents a C₁ to C₄ alkyl, aryl or heteroaryl group, or a —(CH₂)ₘCO₂H or —(CH₂)ₘNR₇R₈ group where m represents an integer of between 1 and 3, and
R₁₇ represents a hydrogen atom or a C₁ to C₄ alkyl or aryl group, and the pharmaceutically acceptable salts thereof and isomers thereof including the enantiomers and mixtures of isomers in any proportion,
with the exception of the following compounds:

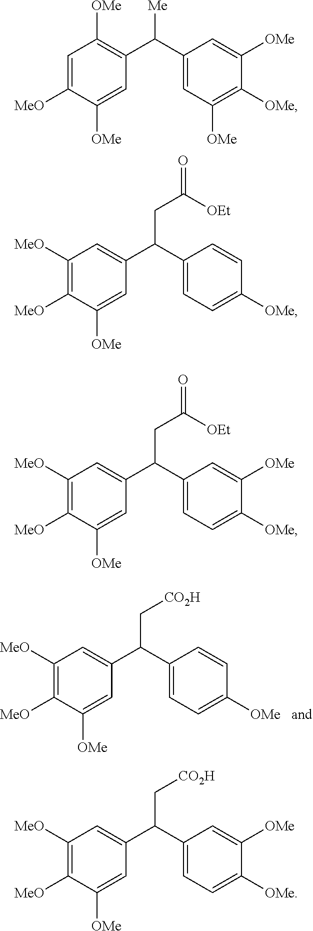

2. The compound according to claim 1, characterized in that R₄ represents a hydrogen atom and R₁, R₂ and R₃ each independently represent a methoxy group optionally substituted with one or more fluorine atoms, and advantageously a methoxy group.

3. The compound according to either of claims 1 and 2, characterized in that the anti-vascular molecule is chosen from among 6-mercaptopurine, fludarabine, cladribine, pentostatin, cytarabine, 5-fluorouracil, gemcitabine, methotrexate, raltitrexed, irinotecan, topotecan, etoposide, daunorubicin, doxorubicin, epirubicin, idarubicin, pirarubicin, mitoxantrone, chlormethine, cyclophosphamide, ifosfamide, melphalan, chlorambucil, busulfan, carmustine, fotemustine, streptozocin, carboplatin, cisplatin, oxaliplatin, procarbazine, dacarbazine, bleomycin, vinblastine, vincristine, vindesine, vinorelbine, paclitaxel, docetaxel, L-asparaginase, flutamide, nilutamide, bicalutamide, cyproterone acetate, triptorelin, leuprorelin, goserelin, buserelin, formestan, aminoglutethimide, anastrazole, letrozole, tamoxifene, octreotide, lanroetide, (Z)-3-[2,4-dimethyl-5-(2-oxo-1,2-dihydro-indol-3-ylidenemethyl)-1H-pyrrol-3-yl]-propionic acid, 4-((9-chloro-7-(2,6-difluorophenyl)-5H-pyrimidol(5,4-d)(2)benzazepin-2-yl)amino)benzoic acid, 5,6-dimethylxanthenone-4-acetic acid or even 3-(4-(1,2-diphenylbut-1-enyl)phenyl)acrylic acid.

4. The compound according to any of claims 1 to 3, characterized in that A is a cycle chosen from the group comprising the groups phenyl, naphtyl, quinolyl, isoquinolyl, imidazolyl, indolyl, benzothiophenyl, benzofuranyl, benzoimidazolyl, purinyl, pyridinyl, pyridazinyl, pyrimidinyl, pyrazinyl, pyrrolyl, furanyl and thiophenyl, and in particular the phenyl, naphtyl, purinyl, benzofuranyl, pyridinyl, quinolyl and indolyl groups,
said cycle possibly being substituted with one or more groups chosen from among -Me, -Bn, —C₆H₄—OMe, —CH₂—C₆H₄—OMe, —(CH₂)₂—C₆H₄—OMe, —(CH₂)₂—C₆H₂—(OMe)₃, —OH, —OMe, —OBn —OCOMe, —C₆H₄NH₂, —OC₆H₄NH₂, —NH₂, —OCONEt₂, —(CH₂)ₓ—OH where x=3, 4, 5 or 6, —OCOCH₂NMe₂, —OPO₃H₂, —F and 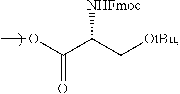
or possibly being fused to a heterocycle of formula 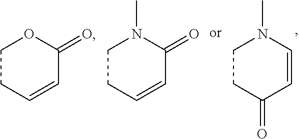
the dotted line representing the common bond between the heterocycle and said cycle.

5. The compound according to any of claims 1 to 4, characterized in that it meets the following formula (Ia): 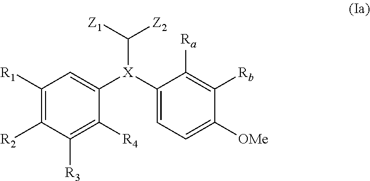
or a pharmaceutically acceptable salt or isomer thereof,
wherein:
R₁, R₂, R₃, R₄, X, Z₁ and Z₂ are such as defined in claim 1,
Rₐ represents a hydrogen or halogen atom, or a group —B(OH)₂, C₁ to C₄ alkyl, C₂ to C₄ alkenyl, C₂ to C₄ alkynyl, aryl, heteroaryl, —COOH, —NO₇, —NR₇R₈, —NHCOR₇, —CONR₇R₈, —NHCOOR₉, —OSi(C₁ to C₄ alkyl)₃, —NHSO₂R₉, C₁ to C₄ alcoxy optionally substituted with one or more fluorine atoms, —OCONR₇R₈, —OSO₂CF₃, —OSO₂R₉, —SO₂R₉, —SO₃R₉, —OSO₃H, —OPO(OR₁₀)₂, —ONR₇R₈, —OR₁₁, —SO₂NR₁₂R₁₃, —SO₂NHCOR₁₄, —OCOR₁₅, —OCOOR₁₆ or —SR₁₇, and advantageously represents a hydrogen atom, and
R_{b} represents a halogen atom, and preferably a fluorine atom, a group aryloxy —OR₁₁, —OCOR₁₅, —OCOOR₁₅, —OCONR₇R₈, —OSO₂R₉, —OSO₂CF₃, —OSO₃H, —OPO(OR₁₀)₂, —ONR₇R₈, —NR₇R₈, —NHCOR₇, —NHCOOR₉, —NHSO₂R₉ or a residue of an anti-vascular molecule bound via an ester or amide bond,
the aryl nuclringsei of said Rₐ and R_{b} groups optionally being substituted with one or more OH, C₁ to C₄ alcoxy, NR₇R₈ groups,
R₇, R₈, R₉, R₁₀, R₁₁, R₁₂, R₁₃, R₁₄ and R₁₅ being such as defined in claim 1.

6. The compound according to any of claims 1 to 5 characterized in that it is chosen from among:

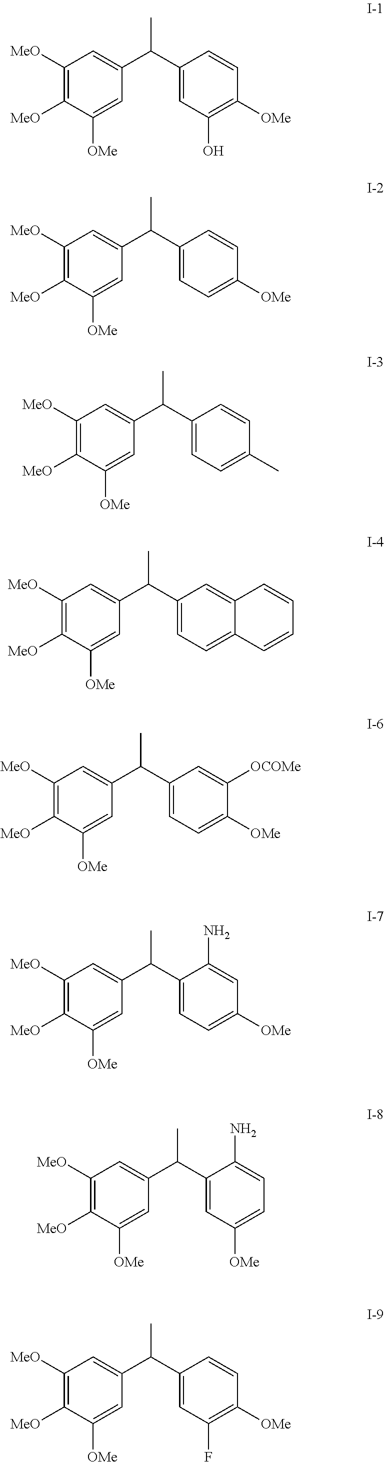

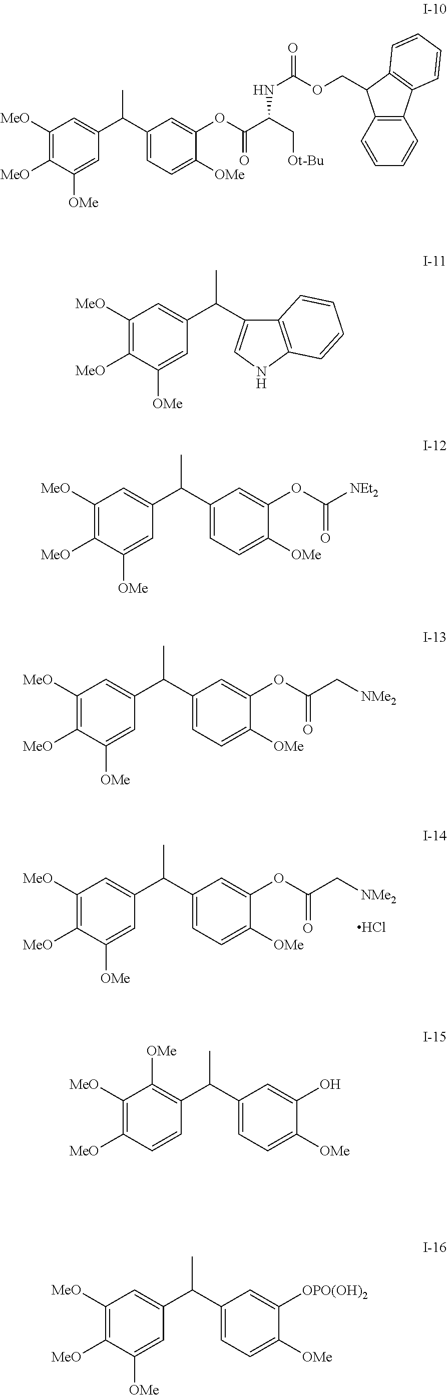

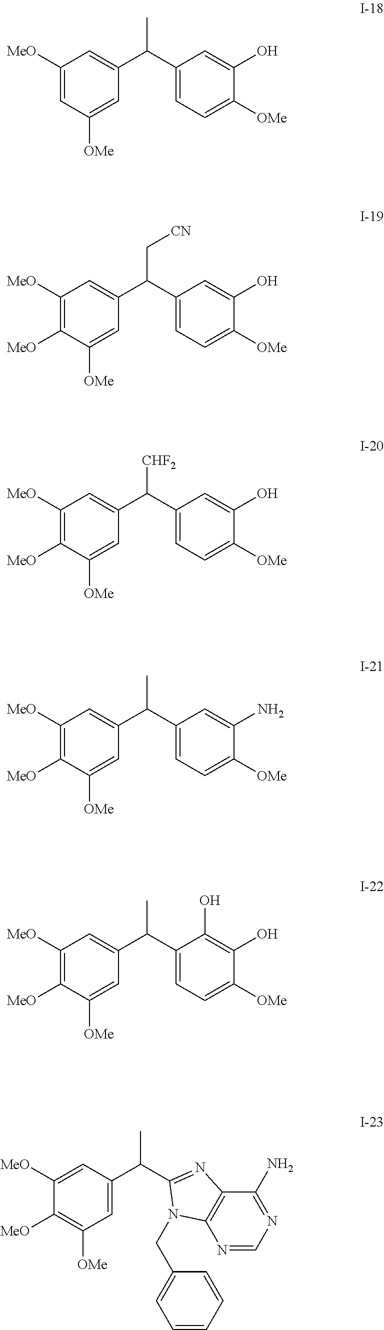

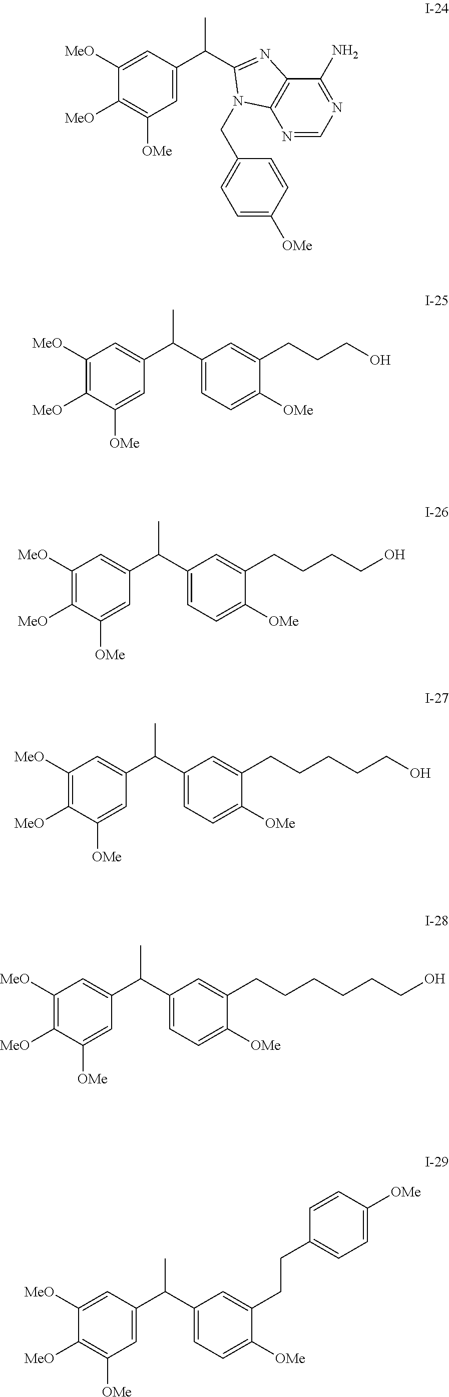

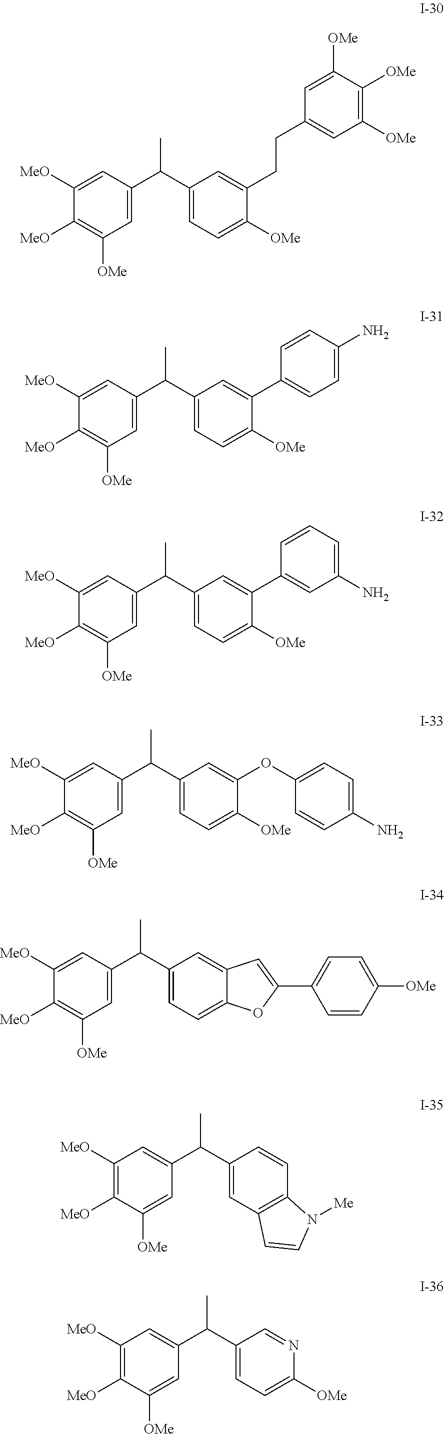

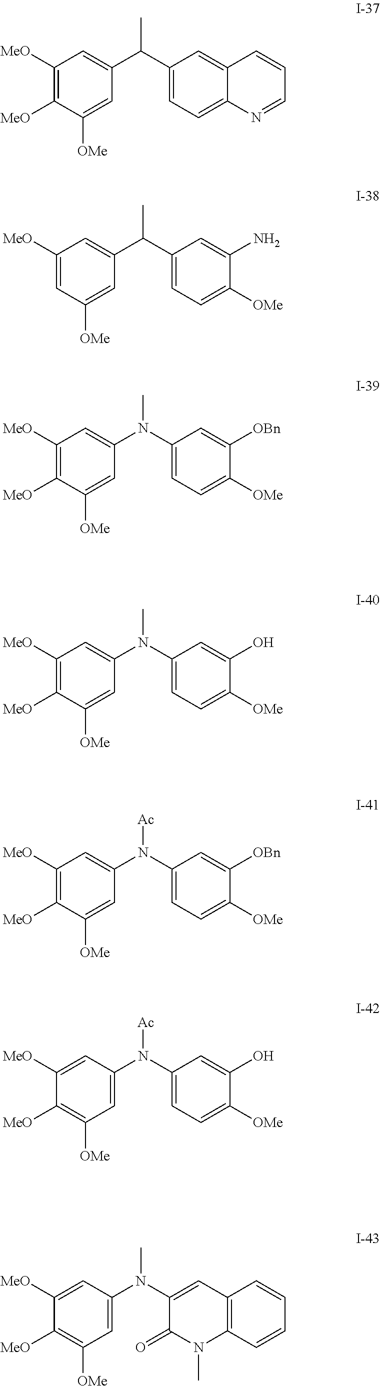

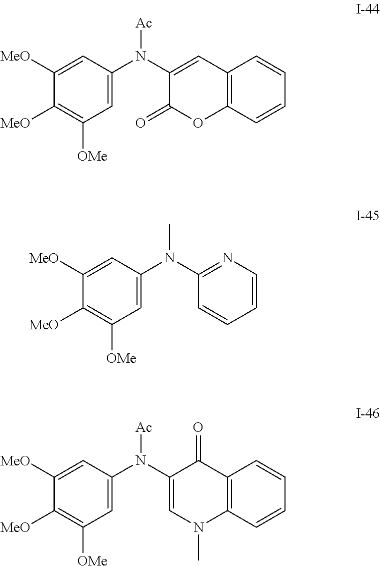

7. Method for preparing a compound of formula (I) such as defined in claim 1, wherein X represents a CH group characterized in that it comprises the following successive steps:
hydrogenation of a compound of following formula (II): 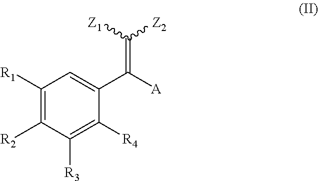
wherein R₁, R₂, R₃, R₄, A, Z₁ and Z₂ are such as defined in claim 1, and
separation of the compound (I) formed at the preceding step from the reaction medium.

8. The method for preparing a compound of formula (I) such as defined in claim 1, wherein X represents a nitrogen atom, characterized in that it comprises the following successive steps:
reacting a compound of following formula (IX): 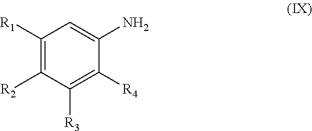
wherein R₁, R₂, R₃ and R₄ are such as defined in claim 1,
with a compound of formula A-Hal, wherein A is such as defined in claim 1 and Hal represents a halogen atom, preferably a bromine, in the presence of a catalyst and a base,
to give a compound of following formula (X): 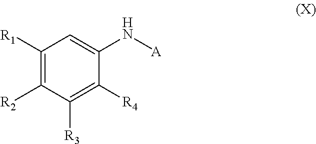
wherein R₁, R₂, R₃, R₄ and A are such as defined in claim 1,
reacting the compound of formula (X) obtained at the preceding step with a compound of formula Z₁Z₂CH—X1, wherein Z₁ and Z₂ are such as defined in claims 1 and X1 represents a halogen atom, in the presence of a base to form a compound of formula (I), and
separation of the compound (I) formed at the preceding step from the reaction medium.

9. The compound of formula (I) according to any of claims 1 to 6, including a compound of formula: 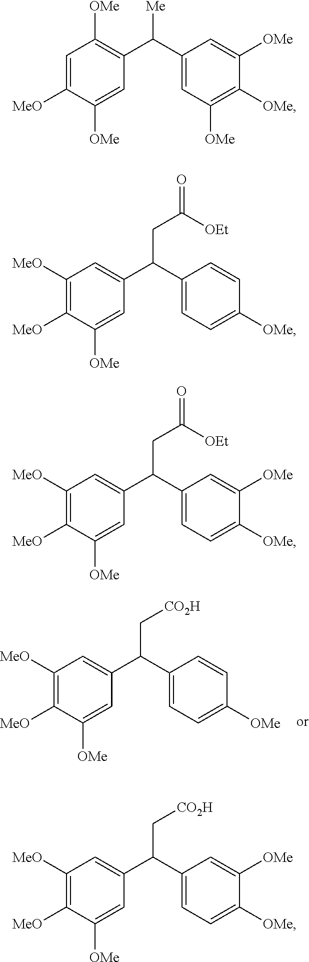
for use thereof as medicament, notably as inhibitor of tubulin polymerization.

10. The compound according to claim 9, for use thereof as medicament intended to treat or prevent proliferative diseases such as cancer, psoriasis or fibrosis.

11. Pharmaceutical composition comprising at least one compound of formula (I) according to any of claims 1 to 6, including a compound of formula: 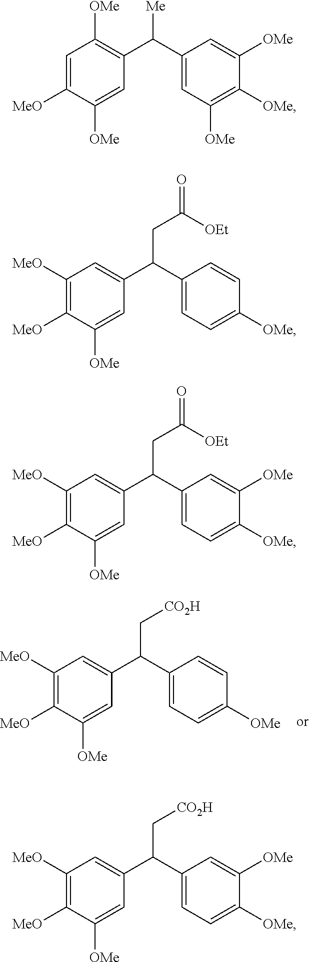
combined with one or more pharmaceutically acceptable excipients.

12. The pharmaceutical composition according to claim 11, characterized in that it comprises at least one other active ingredient, advantageously chosen from among 6-mercaptopurine, fludarabine, cladribine, pentostatin, cytarabine, 5-fluorouracil, gemcitabine, methotrexate, raltitrexed, irinotecan, topotecan, etoposide, daunorubicin, doxorubicin, epirubicin, idarubicin, pirarubicin, mitoxantrone, chlormethine, cyclophosphamide, ifosfamide, melphalan, chlorambucil, busulfan, carmustine, fotemustine, streptozocin, carboplatin, cisplatin, oxaliplatin, procarbazine, dacarbazine, bleomycin, vinblastine, vincristine, vindesine, vinorelbine, paclitaxel, docetaxel, L-asparaginase, flutamide, nilutamide, bicalutamide, cyproterone acetate, triptorelin, leuprorelin, goserelin, buserelin, formestan, aminoglutethimide, anastrazole, letrozole, tamoxifene, octreotide and lanroetide.

13. Pharmaceutical composition comprising:
(i) at least one compound of formula (I) according to any of claims 1 to 6, including a compound of formula: 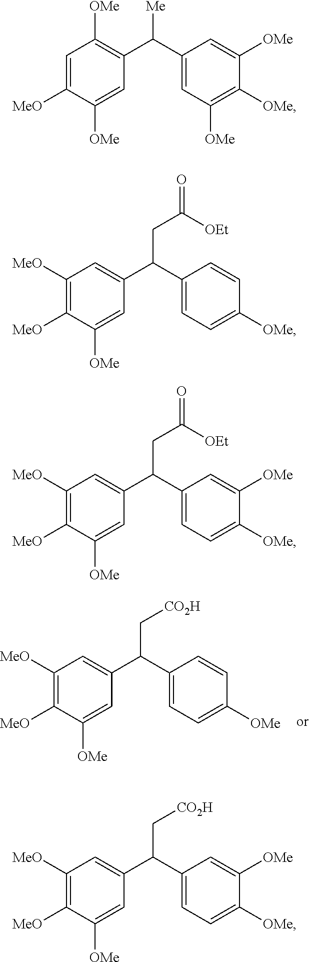
and
(ii) at least one other active ingredient, as combination products for simultaneous, separate or sequential use.

14. The composition according to claim 13, characterized in that the active principle(s) are chosen from among 6-mercaptopurine, fludarabine, cladribine, pentostatin, cytarabine, 5-fluorouracil, gemcitabine, methotrexate, raltitrexed, irinotecan, topotecan, etoposide, daunorubicin, doxorubicin, epirubicin, idarubicin, pirarubicin, mitoxantrone, chlormethine, cyclophosphamide, ifosfamide, melphalan, chlorambucil, busulfan, carmustine, fotemustine, streptozocin, carboplatin, cisplatin, oxaliplatin, procarbazine, dacarbazine, bleomycin, vinblastine, vincristine, vindesine, vinorelbine, paclitaxel, docetaxel, L-asparaginase, flutamide, nilutamide, bicalutamide, cyproterone acetate, triptorelin, leuprorelin, goserelin, buserelin, formestan, aminoglutethimide, anastrazole, letrozole, tamoxifene, octreotide and lanroetide.

15. The composition according to any of claims 11 to 14 for use thereof as medicament, notably intended to treat or prevent proliferative diseases such as cancer, psoriasis or fibrosis.
